(19) United States
(12) Patent Application Publication
(10) Pub. No.: US 2026/0242371 A1
(43) Pub. Date: Aug. 20, 2026

(54) LIPID PRODRUGS OF BROMOLYSERGIDE AND USES THEREOF

(71) Applicant: Seaport Therapeutics, Inc., Seaport Boston, MA (US)

(72) Inventors: Jamie Simpson, Chestnut Hill, MA (US); Tim Quach, Southbank (AU); Daniel Kenneth Bonner, Sharon, MA (US); Michael C. Chen, Norwood, MA (US); Michaela Anne Gold, Somerville, MA (US)

(73) Assignee: Seaport Therapeutics, Inc., Seaport Boston, MA (US)

(21) Appl. No.: 19/642,536

(22) Filed: Apr. 8, 2026

Related U.S. Application Data
(63) Continuation of application No. PCT/US2024/050542, filed on Oct. 9, 2024.
(60) Provisional application No. 63/589,107, filed on Oct. 10, 2023.
(60) Provisional application No. 63/631,812, filed on Apr. 9, 2024.

(51) Int. Cl. C07D 457/06; A61K 31/48; A61K 47/54

(52) U.S. Cl. CPC C07D 457/06; A61K 31/48; A61K 47/542

(57) ABSTRACT

Disclosed herein are lipid prodrugs of bromolysergide (2-bromo-LSD) (2-bromo-lysergic acid diethylamide) that achieve improved pharmacokinetic properties and allow easier chronic dosing of patients. The disclosed lipid prodrugs are suitable for the treatment of cluster headache and mood disorders in human subjects.

### CROSS-REFERENCE TO RELATED APPLICATIONS

This application is a continuation of International Application No. PCT/US2024/050542, filed Oct. 9, 2024, which claims the benefit of priority to U.S. Provisional Application No. 63/589,107, filed Oct. 10, 2023, and U.S. Provisional Application No. 63/631,812, filed Apr. 9, 2024, the entireties of which are incorporated herein by reference.

### FIELD OF INVENTION

The present disclosure relates generally to lymph-directed lipid prodrugs of bromolysergide (2-bromo-lysergic acid diethylamide) with improved pharmacokinetic properties.

### BACKGROUND

There are few effective treatment options available to treat pain associated with a cluster headache. Cluster headache causes severe unilateral temporal or periorbital pain, lasting 15 to 180 minutes and accompanied by autonomic symptoms in the nose, eyes, and face (Weaver-Agostoni, Am Fam Physician., 2013). Serotonin receptor agonists (e.g., sumatriptan, methysergide, dihydroergotamine, and psilocybin) have demonstrated efficacy in cluster headache, but hallucinations, fibrotic side effects, and/or short duration of action prevent their widespread use.

Bromolysergide (2-bromo-LSD) is an ergot alkaloid derivative that is reported to be non-hallucinogenic because of its partial agonism at the 5-HT_{2A} receptor (Lewis M et al., Cell Reports, 2023). However, bromolysergide (2-bromo-LSD) has variable pharmacokinetic properties when administered orally, thereby limiting its potential therapeutic use.

### SUMMARY OF INVENTION

Described herein are lymph-directed lipid prodrugs of bromolysergide (2-bromo-lysergic acid diethylamide) (2-bromo-LSD) with improved properties, such as pharmacokinetic properties, allowing for the treatment of patients with cluster headache, migraine, anxiety, and/or mood disorders.

In one aspect, disclosed herein are lipid prodrugs of bromolysergide (2-bromo-LSD) represented by Formula (I):

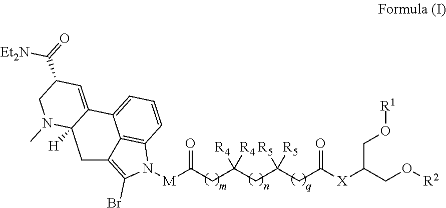

or a pharmaceutically acceptable salt thereof, wherein:
R¹ and R² are each independently hydrogen or —C(O)R³;
   each R³ is independently a saturated or unsaturated, straight or branched, optionally substituted C₂₋₃₇ hydrocarbon chain;
X is —O—;
   R⁴ and R⁵ are each independently hydrogen or C₁₋₄ aliphatic optionally substituted with 1, 2, 3, 4, 5, or 6 deuterium or halogen atoms, or R⁴ and R⁵ taken together with the carbon to which they are attached form a C₃-C₈ cycloalkyl;
M is absent or a self-immolative group;
   n is 0-18; and
   m and q are each independently 0-6.

In some embodiments, R¹ and R² are each independently —C(O)R³. In some embodiments, each R³ is independently a saturated or unsaturated, straight or branched, optionally substituted C₃₋₁₇ hydrocarbon chain. In some embodiments, each R³ is a C₇ hydrocarbon chain. In some embodiments, X is O. In some embodiments, m and q are each independently 0 or 1. In some embodiments, n is 0-6. In some embodiments, R⁴ and R⁵ are each independently hydrogen or methyl.

In some embodiments, M is selected from M is selected from

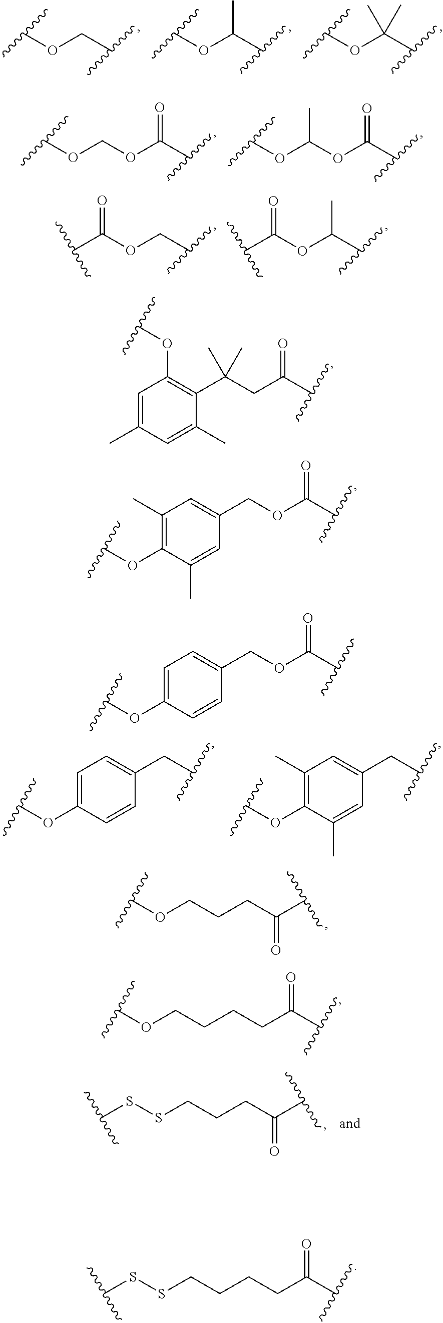

In one aspect, disclosed herein is a compound of Formula II:

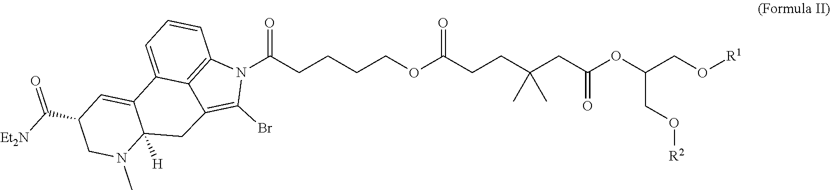

wherein R¹ and R² are each independently —C(O)R³; and
each R³ is independently a saturated or unsaturated, straight or branched, optionally substituted C₃₋₁₇ hydrocarbon chain.

In some embodiments, each R³ is independently a saturated or unsaturated, straight C₃₋₁₇ hydrocarbon chain. In some embodiments, each R³ is a saturated C₇ hydrocarbon chain.

In some embodiments, the compound is I-19:

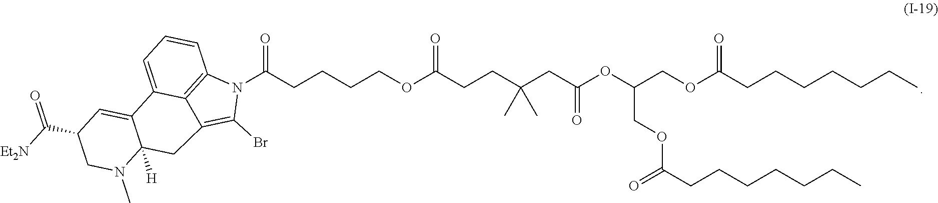

In one aspect, disclosed herein is a compound of Formula III:

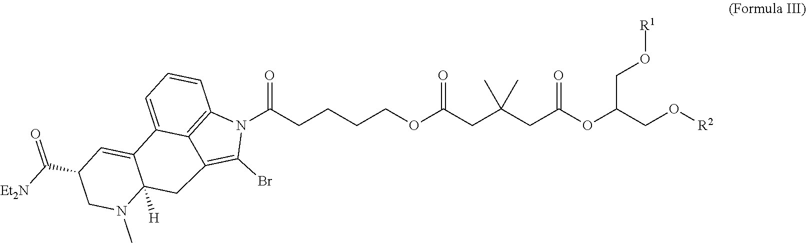

wherein R¹ and R² are each independently —C(O)R³; and
each R³ is independently a saturated or unsaturated, straight or branched, optionally substituted C₃₋₁₇ hydrocarbon chain.

In some embodiments, each R³ is independently a saturated or unsaturated, straight C₃₋₁₇ hydrocarbon chain. In some embodiments, each R³ is a saturated C₇ hydrocarbon chain.

In some embodiments, the compound is I-16:

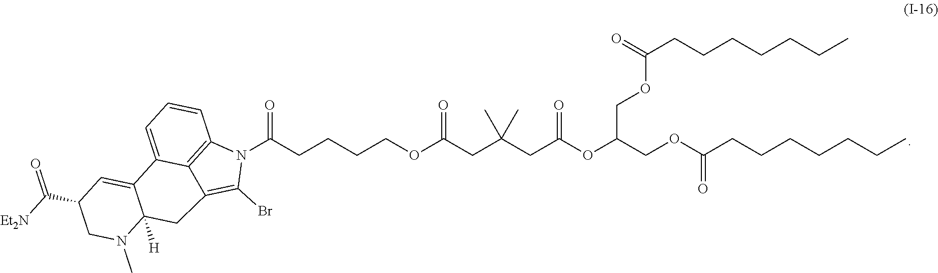

In one aspect, disclosed herein is a compound of Formula IV:

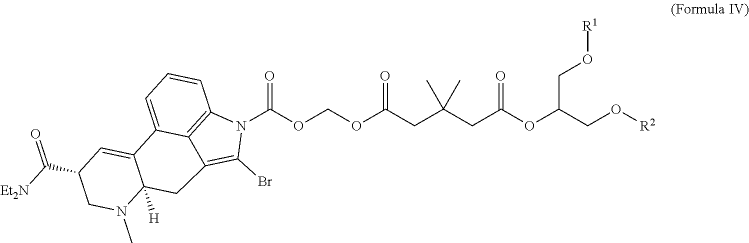

wherein R¹ and R² are each independently —C(O)R³; and
each R³ is independently a saturated or unsaturated, straight or branched, optionally substituted C₃₋₁₇ hydrocarbon chain.

In some embodiments, each R³ is independently a saturated or unsaturated, straight C₃₋₁₇ hydrocarbon chain. In some embodiments, each R³ is a saturated C₇ hydrocarbon chain.

In some embodiments, the compound is I-9:

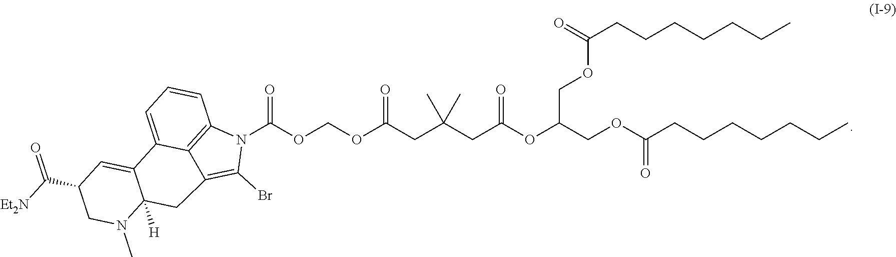

In one aspect, disclosed herein is a compound of Formula V:

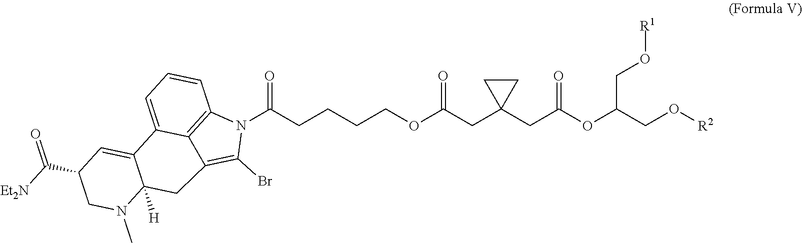

wherein R¹ and R² are each independently —C(O)R³; and
each R³ is independently a saturated or unsaturated, straight or branched, optionally substituted C₃₋₁₇ hydrocarbon chain.

In some embodiments, each R³ is independently a saturated or unsaturated, straight C₃₋₁₇ hydrocarbon chain. In some embodiments, each R³ is a saturated C₇ hydrocarbon chain.

In some embodiments, the compound is I-18:

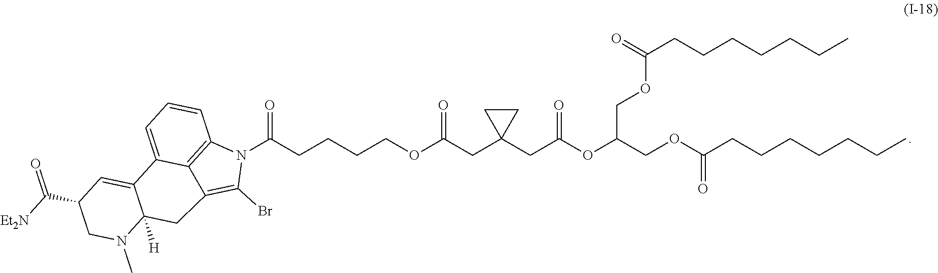

In one aspect, disclosed herein is a compound of Formula VI:

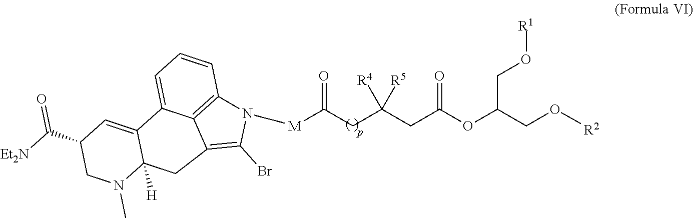

wherein R¹ and R² are each independently —C(O)R³;
each R³ is independently a saturated or unsaturated, straight or branched, optionally substituted C₃₋₁₇ hydrocarbon chain;
R⁴ and R⁵ are each independently hydrogen or C₁₋₄ aliphatic optionally substituted with 1, 2, 3, 4, 5, or 6 deuterium or halogen atoms, or R⁴ and R⁵ taken together with the carbon to which they are attached form a C₃-C₈ cycloalkyl;
p is 1 or 2; and
-M- is

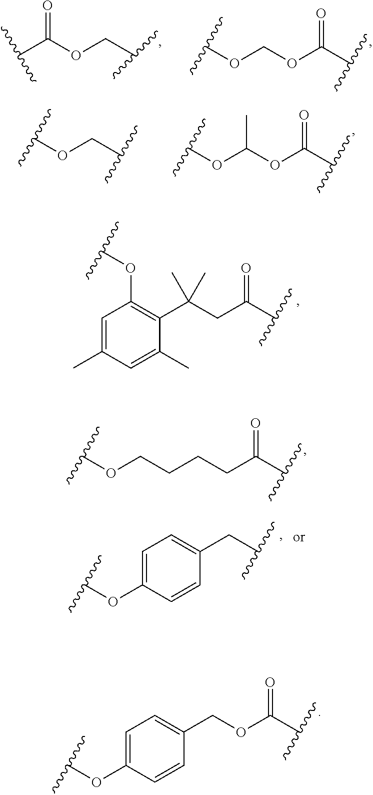

In some embodiments, each R³ is independently a saturated or unsaturated, straight C₃₋₁₇ hydrocarbon chain. In some embodiments, each R³ is a saturated C₇ hydrocarbon chain. In some embodiments, -M- is

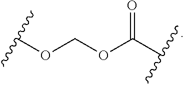 [SMILES: *OCOC(*)=O]

In some embodiments, -M- is

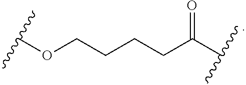 [SMILES: *OCCCCC(*)=O]

In some embodiments, R⁴ and R⁵ taken together with the carbon to which they are attached form a C₃ cycloalkyl. In some embodiments, R⁴ is methyl and R⁵ is hydrogen. In some embodiments, R⁴ and R⁵ are each methyl. In some embodiments, p is 1. In some embodiments, p is 2.

In another aspect, the present disclosure is directed to pharmaceutically acceptable composition comprising the compound of any one of formulae (I), (II), (III), (IV), (V), or (VI) and a pharmaceutically acceptable carrier, adjuvant, or vehicle.

In some embodiments, the pharmaceutically acceptable composition is administered orally to a subject in need thereof.

In a further aspect, the present disclosure is directed to a method of treating cluster headache, migraine, an anxiety disorder, or a mood disorder in a patient in need thereof comprising administering to the patient the compound of any one of formulae (I), (II), (III), (IV), (V), or (VI) or a pharmaceutically acceptable composition comprising the compound of any one of formulae (I), (II), (III), (IV), (V), or (VI). In some embodiments, the mood disorder is depression, bipolar disorder, premenstrual syndrome dysphoric disorder (PMDD), intermittent explosive disorder (IED), or substance-induced mood disorder.

In some embodiments, the method further comprises the step of administering an additional therapeutic agent.

### BRIEF DESCRIPTION OF THE DRAWINGS

FIG. 1 is a graphical illustration of a metabolic scheme of bromolysergide (2-bromo-LSD) in human liver microsomes (HLM) and rat and human hepatocytes (R and H, respectively). Gluc=glucuronide and GSH=glutathione.

FIGS. 2A-D demonstrate the variability in pharmacokinetic profiles observed in non-human primates for an aqueous cyclodextrin formulation of 2-bromo-LSD (2-BL) (FIG. 2A), a self-emulsifying drug delivery system (SEDDS) lipid formulation of 2-bromo-LSD (FIG. 2B), and SEDDS lipid prodrugs of Formula I (FIG. 2C-2D).

FIG. 3 demonstrates the shift in Tmax for SEDDS lipid prodrugs of Formula I as compared to 2-bromo-LSD.

FIG. 4 demonstrates the sex difference in AUC observed for a SEDDS lipid formulation of 2-bromo-LSD as compared to SEDDS lipid prodrugs of Formula I.

### DETAILED DESCRIPTION

Disclosed herein are lymph-directed lipid prodrugs of bromolysergide (2-bromo-lysergic acid diethylamide) (2-bromo-LSD) with improved pharmacokinetic and/or tolerability properties. The improved pharmacokinetic and/or tolerability properties may allow, in part, chronic dosing of bromolysergide (2-bromo-LSD) with reduced central nervous system (CNS) effects.

### Lymphatic System-Directing Prodrugs

Lipid prodrugs of the present disclosure and compositions thereof are useful in promoting transport of bromolysergide (2-bromo-LSD) to the lymphatic system and subsequent release of the parent drug, i.e., the therapeutic agent (e.g., bromolysergide (2-bromo-LSD)).

In one aspect, the present disclosure provides lipid prodrugs of bromolysergide (2-bromo-LSD). Without being bound by any particular theory, it is expected that a prodrug of bromolysergide (2-bromo-LSD) capable of averting first-pass metabolism will have improved pharmacokinetic properties and/or tolerability as compared to bromolysergide (2-bromo-LSD) alone. The lipid prodrugs of the instant disclosure are transported to the lymph, thus avoiding first-pass metabolism in the liver. In bypassing first-pass metabolism, the lipid prodrugs maintain high oral bioavailability of bromolysergide (2-bromo-LSD) for their oral administration to a subject. Lymphatic transport also involves processing via the natural fat-absorption biochemical pathways of the intestine. Transit through these pathways is typically a longer process than standard small molecule absorption, leading to maximal concentration of absorbed dietary lipids in the 4-5 hours after ingestion. Accordingly, in some embodiments the lipid prodrugs disclosed herein can be used to treat a disease or disorder in which elevated levels of bromolysergide (2-bromo-LSD) are beneficial.

### Definitions

While the terms used herein are believed to be well understood by one of ordinary skill in the art, definitions are set forth herein to facilitate explanation of the presently disclosed subject matter.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence. In some embodiments, the disease or disorder is cluster headache, migraine, an anxiety disorder, or a mood disorder.

As used herein, the term “optionally substituted” is taken to mean that a group may or may not be further substituted with one or more groups. Group suitable for optional substitution include, but are not limited to, alkyl, alkenyl, alkynyl, and hydroxyl. Additional groups suitable for optional substitution are described in WO2016/023082, WO2017/041139, and WO2019/046491, the entireties of which are hereby incorporated by reference.

The term “aliphatic” or “aliphatic group,” as used herein, means a straight-chain (i.e., unbranched) or branched, substituted or unsubstituted hydrocarbon chain that is completely saturated or that contains one or more units of unsaturation. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. Examples of aliphatic groups, include but are not limited to methyl, ethyl, n-propyl, i-propyl, n-butyl, t-butyl, sec-butyl, and iso-butyl groups.

The term “halogen” means F, Cl, Br, or I.

The term “self-immolative group,” as used herein, refers to a bivalent chemical moiety that comprises a covalent, scissile bond as one of its bivalent bonds and a stable, covalent bond with a therapeutic agent as its other bivalent bond, wherein the bond with the therapeutic agent becomes labile upon cleavage of the scissile bond. Examples of self-immolative groups include, but are not limited to, disulfide self-immolative groups, hydrazone self-immolative groups, acetal self-immolative groups, carboxyacetal self-immolative groups, carboxy(methylacetal) self-immolative groups, p-hydroxybenzyl self-immolative groups, para-hydroxybenzyl carbonyl self-immolative groups, flipped ester self-immolative groups, and trimethyl lock, or 2-hydroxyphenyl carbamate (2-HPC) self-immolative groups. A number of other suitable self-immolative groups are known in the art as described, for example, in C. A. Blencowe et al., Polym. Chem. 2011, 2, 773-790 and F. Kratz et al., ChemMedChem. 2008, 3 (1), 20-53; Huvelle, S. et al., Org. Biomol. Chem. 2017, 15 (16), 3435-3443; and Alouane, A. et al., Angew. Chem. Int. Ed. 2015, 54 (26), 7492-7509; and Levine, M. N. et al., Chem. Sci. VL-IS-3 (8), 2412-2420; each of which is hereby incorporated by reference in its entirety.

Lipid Prodrugs of Bromolysergide (2-bromo-LSD)

The development of prodrugs is often employed to improve the pharmacokinetic (PK) and pharmacodynamic (PD) properties of a therapeutic agent, including potential side effects associated with a given PK profile. While numerous classes of prodrugs exist in the art, lipid prodrugs are particularly effective at addressing oral bioavailability issues. Lipid mimetic compounds are expected to behave similarly to natural triglycerides, enabling transport through the lymphatic system prior to reaching systemic circulation, effectively circumventing first-pass metabolism. Lipid prodrugs are further discussed in WO2016/023082 and WO2017/041139.

Disclosed herein are lipid prodrugs of bromolysergide (2-bromo-LSD) that avoid first-pass metabolism and result in systemic exposure of 2-bromo-LSD.

Bromolysergide (2-bromo-LSD), below structure, was first synthesized in the 1950s and found to be effective for headaches, including migraines (Sicuteri, F., Triangle 1963, 6, 116-125) but has observed central nervous system (CNS) side effects. A structurally related serotonin receptor agonist, methysergide, was commercialized as an effective migraine and cluster headache treatment. However, it was discovered that chronic methysergide use can lead to retroperitoneal and pulmonary fibrosis due to strong 5-HT_{2B} receptor agonism of a major metabolite, and methysergide was discontinued (Koehler, P. et al., Cephalalgia 2008, 28, 1126-1135).

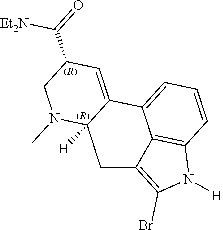

### Bromolysergide (2-bromo-LSD)

Classical psychedelics, like psilocybin, have demonstrated efficacy in cluster headache, but these molecules are also 5-HT_{2B} receptor agonists, posing risks for long-term use, for instance in chronic cluster headache treatment (Schindler, E. A. D. et al., Headache 2022, 62, 1383-1394; Madsen, M. K. et al., medRxiv 2022.07.10.22277414; Tagen, M. et al., Psychopharmacol 2023, 37, 876-890). In contrast, bromolysergide (2-bromo-LSD) is a 5-HT_{2B} antagonist, and its chronic use would not be expected to lead to fibrosis. Another benefit of bromolysergide (2-bromo-LSD) over classical psychedelics is its partial agonism of the 5-HT_{2A} receptor, which does not trigger hallucinogenic effects as stronger agonism does (Lewis, V. et al, Cell Reports 2023, 42, 112203). Patients have been given up to 22 mg of bromolysergide (2-bromo-LSD) with no hallucinations reported (Schneckloth, R., et al., Circulation 1957, 16, 523-532). However, bromolysergide (2-bromo-LSD)'s pharmacokinetic (PK) properties likely play a role in CNS side effects noted in a 2010 cluster headache trial, which may also potentially lead to driving impairment after treatment (Karst, M., et al., Cephalalgia 2010, 30, 1140-1144). The acute CNS side effects in the trial (Karst, M., et al., Cephalalgia 2010, 30, 1140-1144) are likely caused by the maximum serum concentration (Cₘₐₓ) of the drug. In addition, a preclinical study with bromolysergide (2-bromo-LSD) in mice demonstrated sex differences in the PK profile, variable exposure and half-life, and non-linear PK properties (Lewis, V. et al, Cell Reports 2023, 42, 112203). A structurally related compound, dihydroergotamine (DHE), has shown inconsistent efficacy and adverse events due to PK variability across formulations (Silberstein S D et al., “Then and Now: A Narrative Review” Headache 60:45-57 (2020)). PK variability observed in certain headache medications, like amitriptyline and topiramate, require titration to ensure safety and tolerability (Tfelt-Hansen P et al., Pharmacokinetic Variability of Drugs Used for Prophylactic Treatment of Migraine. CNS Drugs 31:389-403 (2017)). Additionally, a pre-clinical study of 2-bromo-LSD demonstrated sex differences in the PK profile, which could lead to sex-specific adverse events in clinical trials (Lewis V et al., “A non-hallucinogenic LSD analog with therapeutic potential for mood disorders” Cell Reports 42, 112-203 (2023) and Zucker I and Prendergast B J “Sex Differences in Pharmacokinetics Predict Adverse Drug Reactions in Women” Biology of Sex Differences 11:32 (2020)). The PK variability and likely link to CNS side effects are a limitation of bromolysergide (2-bromo-LSD) that can be addressed via the lipid prodrugs of bromolysergide (2-bromo-LSD) described herein, which could improve the PK profile, reduce dose, and potentially reduce CNS side effects.

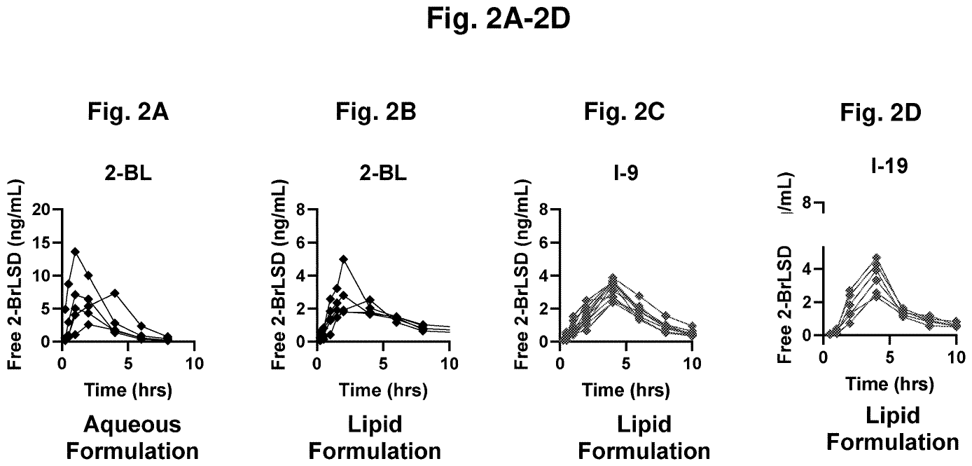

In particular, the lipid prodrugs of 2-bromo-LSD of the present disclosure result in reduced variability of the pharmacokinetic profile as shown in FIG. 2A-2D. FIGS. 2A and 2B demonstrate the variability in PK for 2-bromo-LSD in both aqueous (FIG. 2A) and SEDDS lipid (FIG. 2B) formulations. By contrast, FIGS. 2C and 2D demonstrate the reduced variability in PK afforded by prodrugs of the present disclosure.

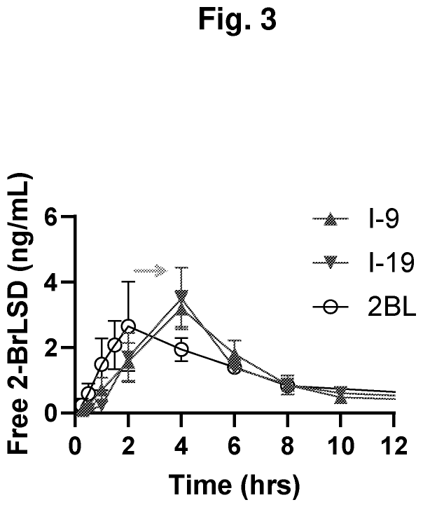

Further, the lipid prodrugs of 2-bromo-LSD result in a shift in the observed Tmax, from ~2 hours for the SEDDS formulation of 2-bromo-LSD (black circles) to ~4 hours for the prodrugs of the present disclosure (triangles) as shown in FIG. 3.

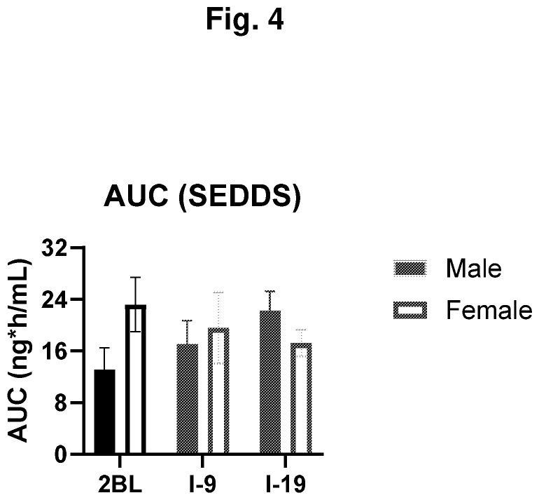

Additionally, the lipid prodrugs of 2-bromo-LSD minimize sex differences in AUC levels that were observed for SEDDS formulations of 2-bromo-LSD. FIG. 4 demonstrates the reduction in sex differences observed with the lipid prodrugs of the present disclosure as compared to a SEDDS formulation of 2-bromo-LSD.

As used herein, the terms “bromolysergide”, “2-bromo-LSD”, and “2BL” are used interchangeably.

Accordingly, in one aspect, disclosed herein are compounds of Formula I:

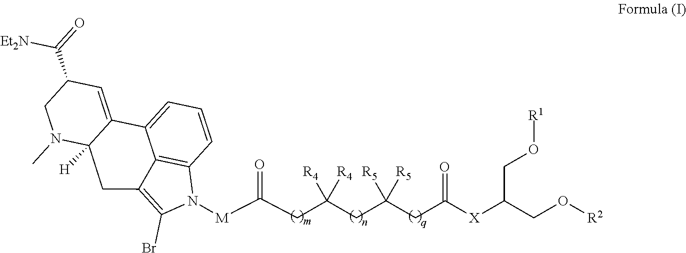

or a pharmaceutically acceptable salt thereof, wherein:
R¹ and R² are each independently hydrogen or —C(O)R³;
   each R³ is independently a saturated or unsaturated, straight or branched, optionally substituted C₂₋₃₇ hydrocarbon chain;
X is —O—;
   R⁴ and R⁵ are each independently hydrogen or C₁₋₄ aliphatic optionally substituted with 1, 2, 3, 4, 5, or 6 deuterium or halogen atoms, or R⁴ and R⁵ taken together with the carbon to which they are attached form a C₃-C₈ cycloalkyl;
M is absent or a self-immolative group;
   n is 0-18; and
   m and q are each independently 0-6.

In some embodiments, R¹ and R² are each independently —C(O)R³.

In some embodiments, each R³ is independently a saturated or unsaturated, straight or branched, optionally substituted C₃₋₁₇ hydrocarbon chain. In some embodiments, each R³ is a C₁₇ hydrocarbon chain. In some embodiments, each R³ is a C₇ hydrocarbon chain.

In some embodiments, X is O.

In some embodiments, m and q are each independently 0 or 1.

In some embodiments, n is 0-6. In further embodiments, n is 0.

In some embodiments, the sum of n+m+q is 0, 1, 2, 3, 4, 5, 6, 7, 8, or a range between and including any two of these values. In some embodiments, the sum of n+m+q is 1. In some embodiments, n is 0, m is 0, and q is 1.

In some embodiments, R⁴ and R⁵ are each independently hydrogen or methyl.

In some embodiments, M is absent.

In some embodiments, M is selected from

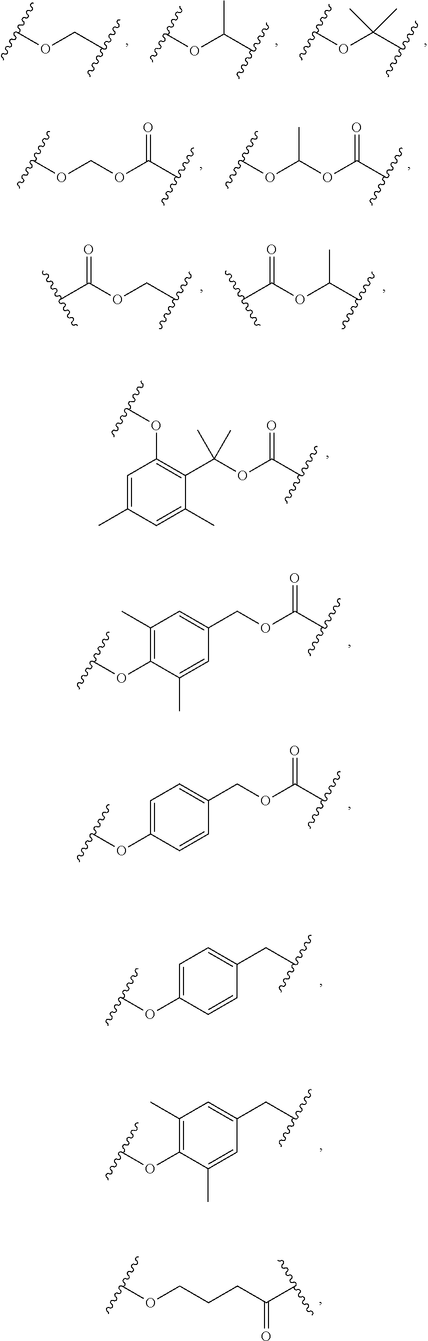

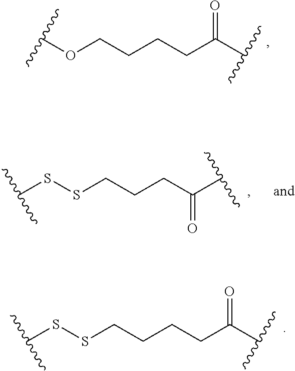 [SMILES: *OCCCCC(*)=O ; *SSCCCC(*)=O ; *SSCCCCC(*)=O]

In some embodiments, M is

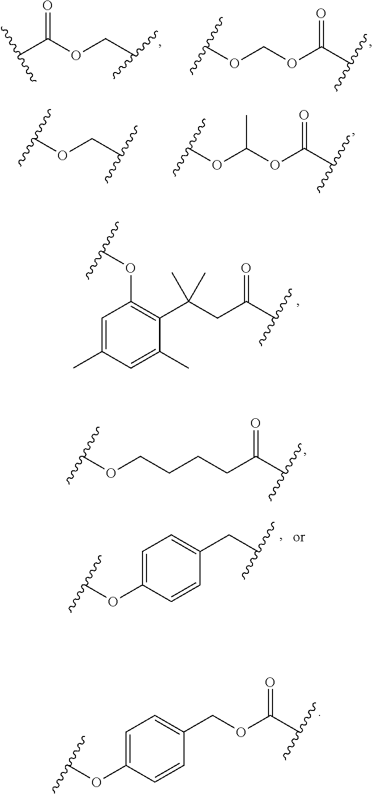

In some embodiments, the compound (CPD) of Formula I is selected from Table 1:

TABLE 1
Exemplary Compounds

| CPD | |
|---|---|
| ID | Compound |
| I-1 | 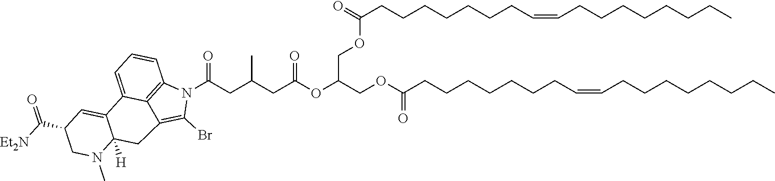 |
| | 2BL-C5bMe-2-TG-oleate |
| I-2 | 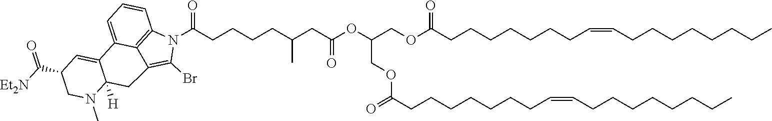 |
| | 2BL-C8bMe-2-TG-oleate |
| I-3 | 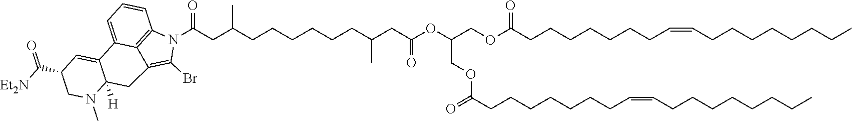 |
| | 2BL-C12b′bMe-2-TG-oleate |
| I-4 | 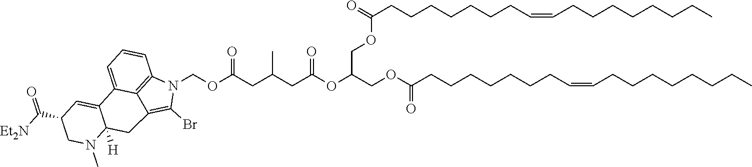 |
| | 2BL-ASI-C5bMe-2-TG-oleate |
| I-5 | 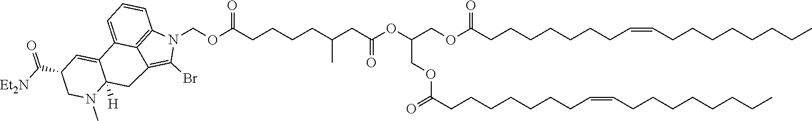 |
| | 2BL-ASI-C8bMe-2-TG-oleate |
| I-6 | 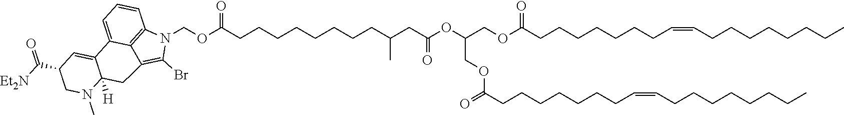 |
| | 2BL-ASI-C12bMe-2-TG-oleate |
| I-7 | 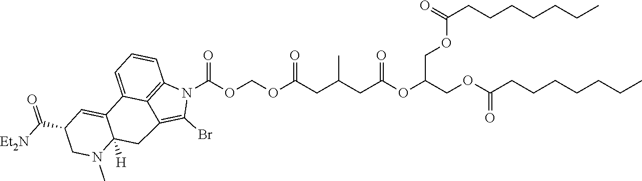 |
| | 2BL-CASI-C5bMe-2-TG-octanoate |
| I-8 | 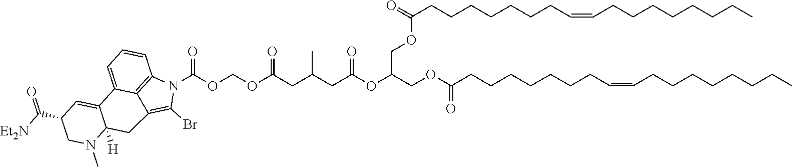 |
| | 2BL-CASI-C5bMe-2-TG-oleate |
| I-9 | 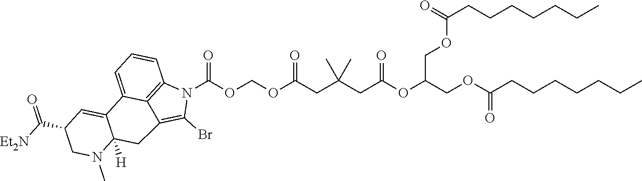 |
| | 2BL-CASI-C5bbGMe-2-TG-octanoate |
| I-9′ | 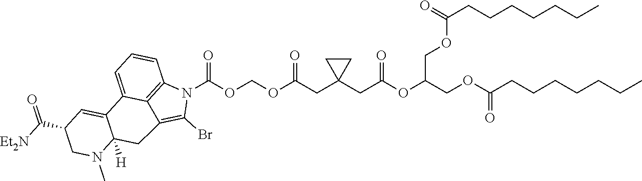 |
| | 2BL-CASI-C5bcPr-2-TG-octanoate |
| I-10 | 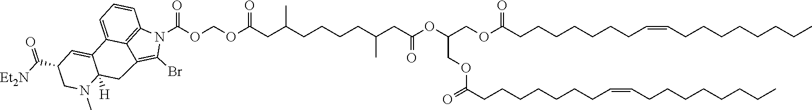 |
| | 2BL-CASI-C10b′bMe-2-TG-oleate |
| I-11 | 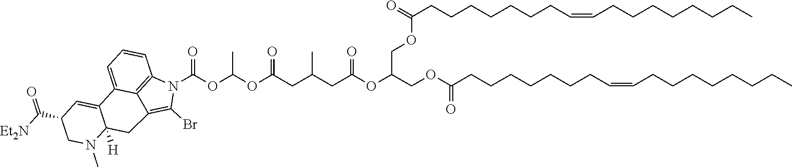 |
| | 2BL-CMSI-C5bMe-2-TG-oleate |
| I-12 | 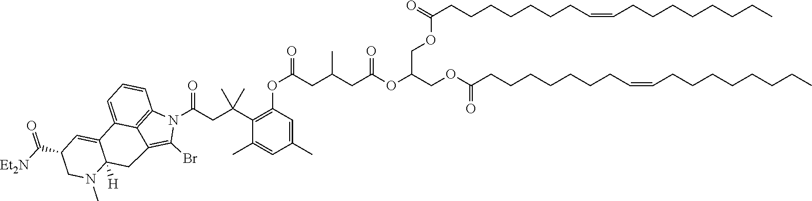 |
| | 2BL-TML-C5bMe-2-TG-oleate |
| I-13 | 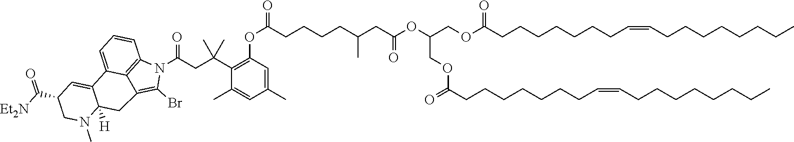 |
| | 2BL-TML-C8bMe-2-TG-oleate |
| I-14 | 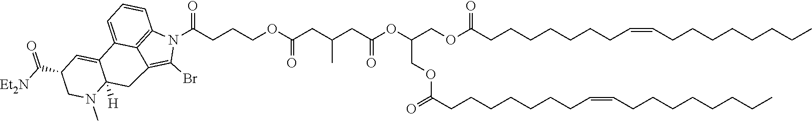 |
| | 2BL-FSI4-C5bMe-2-TG-oleate |
| I-15 | 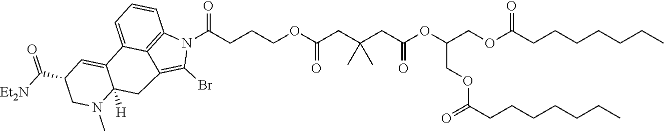 |
| | 2BL-FSI4-C5bbGMe-2-TG-octanoate |
| I-15′ | 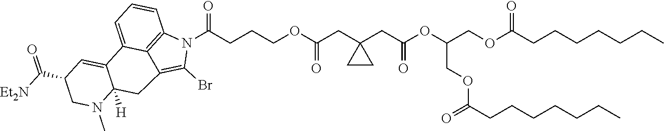 |
| | 2BL-FSI4-C5bcPr-TG-octanoate |
| I-16 | 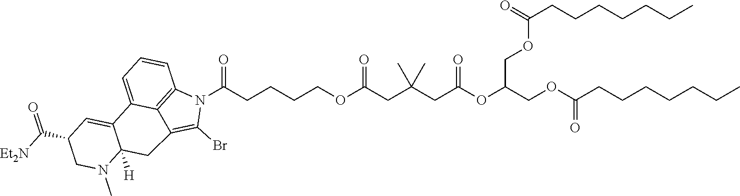 |
| | 2BL-FSI5-C5bbGMe-2-TG-octanoate |
| I-17 | 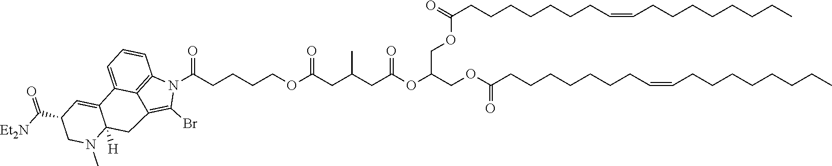 |
| | 2BL-FSI5-C5bMe-2-TG-oleate |
| I-18 | 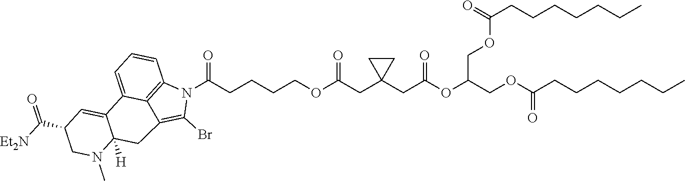 |
| | 2BL-FSI5-C5bcPr-2-TG-octanoate |
| I-19 | 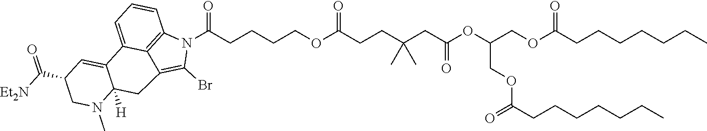 |
| | 2BL-FSI5-C6bbGMe-2-TG-octanoate |
| I-20 | 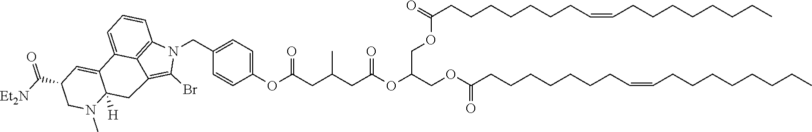 |
| | 2BL-PHB-C5bMe-2-TG-oleate |
| I-21 | 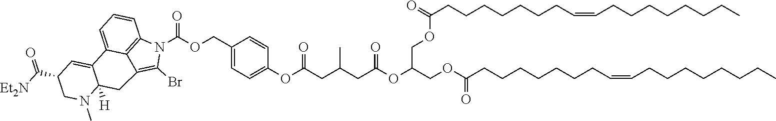 |
| | 2BL-CPHB-C5bMe-2-TG-oleate |
| I-22 | 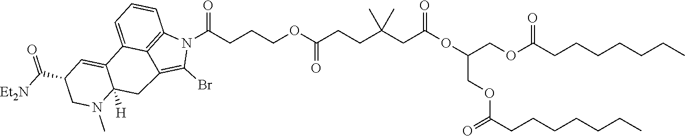 |
| | 2BL-FSI4-C6bbGMe-2-TG-octanoate |
| I-23 | 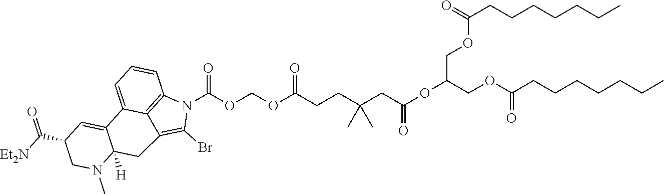 |
| | 2BL-CASI-C6bbGMe-2-TG-octanoate |
| I-24 | 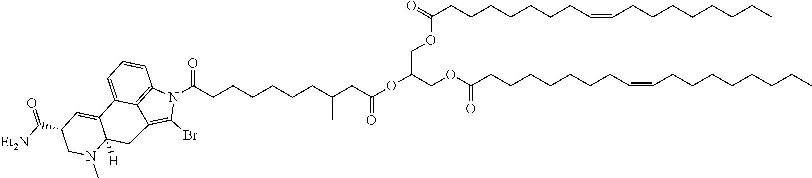 |
| | 2BL-C10bMe-2-TG-oleate |
| I-25 | 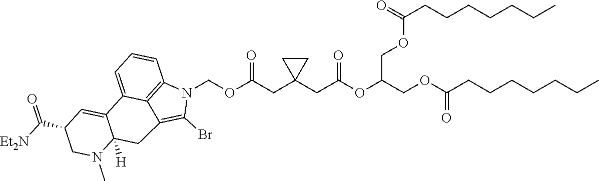 |
| | 2BL-ASI-C5bcPr-2-TG-octanoate |
| I-26 | 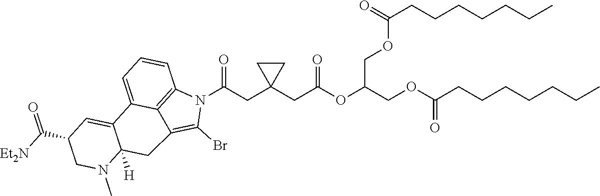 |
| | 2BL-C5bcPr-2-TG-octanoate |
| I-27 | 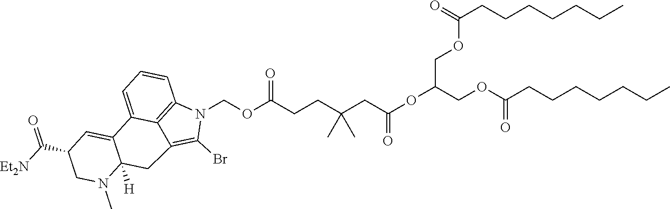 |
| | 2BL-ASI-C6bbGMe-2-TG-octanoate |
| I-28 | 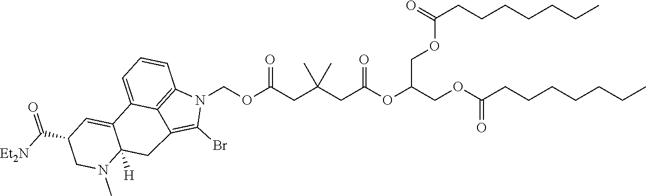 |
| | 2BL-ASI-C5bbGMe-2-TG-octanoate |

or a pharmaceutically acceptable salt thereof.

The above compounds of Formula I are depicted as having either residues of an oleic acid or octanoic acid at the R¹ and R² positions. It is understood that a number of residues of fatty acids are suitable for the compounds disclosed herein. As such, compounds of Formula I could be made with any suitable residue of a fatty acid and be considered within the scope of the instant disclosure. Examples of fatty acids include, but are not limited to, saturated straight-chain fatty acids, saturated branched fatty acids, unsaturated fatty acids, hydroxy fatty acids, and polycarboxylic acids. Fatty acids, and residues thereof, that are suitable for use in the compounds disclosed herein are further described in WO2016/023082, WO2017/041139, and WO2019/046491.

In one aspect, disclosed herein is a compound of Formula II:

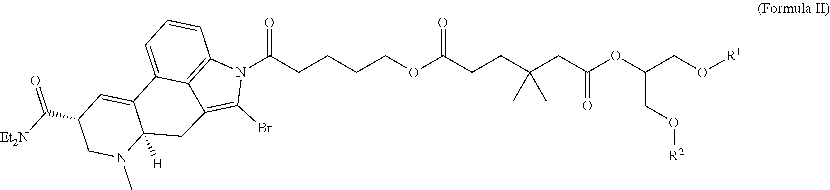

wherein R¹ and R² are each independently —C(O)R³; and
each R³ is independently a saturated or unsaturated, straight or branched, optionally substituted C₃₋₁₇ hydrocarbon chain.

In some embodiments, each R³ is independently a saturated or unsaturated, straight C₃₋₁₇ hydrocarbon chain. In some embodiments, each R³ is a saturated C₇ hydrocarbon chain.

In one aspect, a compound of Formula II is:

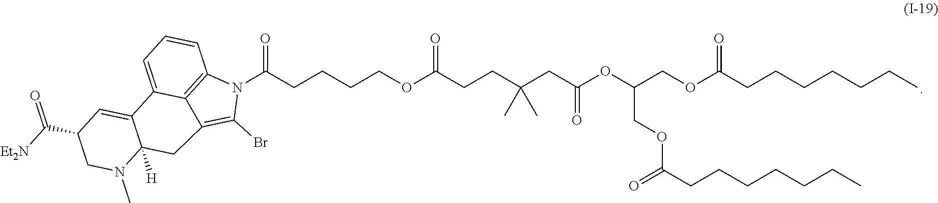

In one aspect, disclosed herein is a compound of Formula III:

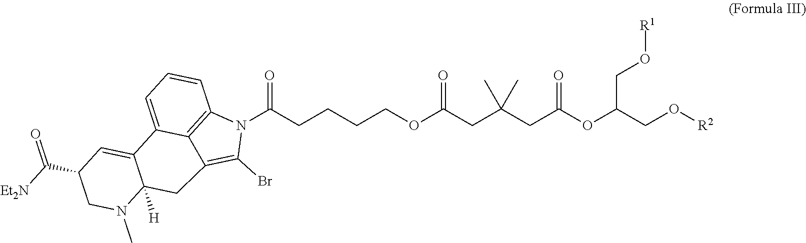

wherein R¹ and R² are each independently —C(O)R³; and
each R³ is independently a saturated or unsaturated, straight or branched, optionally substituted C₃₋₁₇ hydrocarbon chain.

In some embodiments, each R³ is independently a saturated or unsaturated, straight C₃₋₁₇ hydrocarbon chain. In some embodiments, each R³ is a saturated C₇ hydrocarbon chain.

In one aspect, a compound of Formula III is:

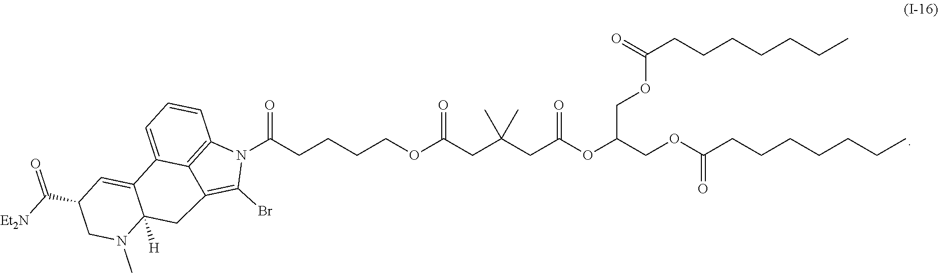

In one aspect, disclosed herein is a compound of Formula IV:

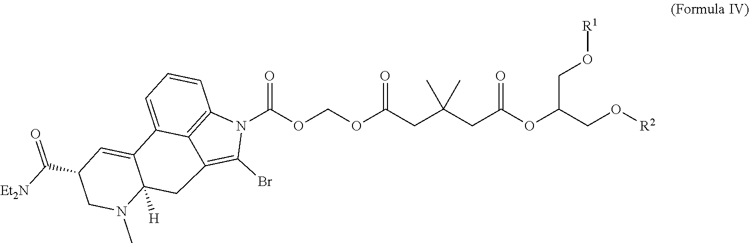

wherein R¹ and R² are each independently —C(O)R³; and
each R³ is independently a saturated or unsaturated, straight or branched, optionally substituted C₃₋₁₇ hydrocarbon chain.

In some embodiments, each R³ is independently a saturated or unsaturated, straight C₃₋₁₇ hydrocarbon chain. In some embodiments, each R³ is a saturated C₇ hydrocarbon chain.

In one aspect, a compound of Formula IV is:

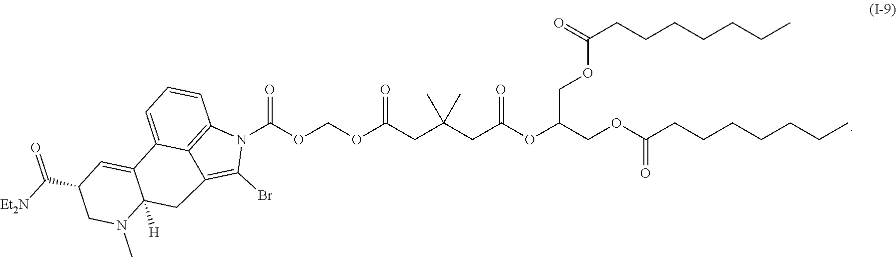

In one aspect, disclosed herein is a compound of Formula V:

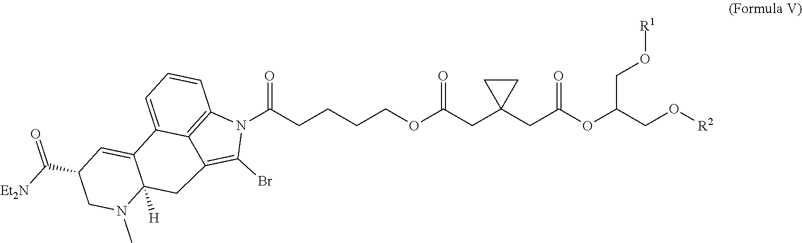

wherein R¹ and R² are each independently —C(O)R³; and
each R³ is independently a saturated or unsaturated, straight or branched, optionally substituted C₃₋₁₇ hydrocarbon chain.

In some embodiments, each R³ is independently a saturated or unsaturated, straight C₃₋₁₇ hydrocarbon chain. In some embodiments, each R³ is a saturated C₇ hydrocarbon chain.

In one aspect, a compound of Formula Vis:

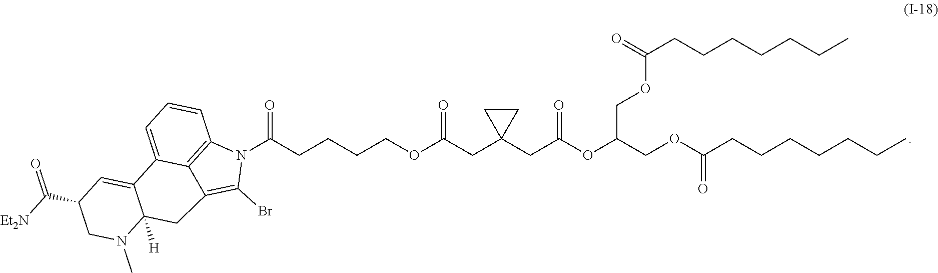

In one aspect, disclosed herein is a compound of Formula VI:

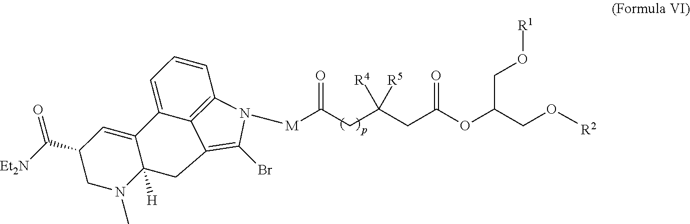

wherein R¹ and R² are each independently —C(O)R³;
each R³ is independently a saturated or unsaturated, straight or branched, optionally substituted C₃₋₁₇ hydrocarbon chain;
R⁴ and R⁵ are each independently hydrogen or C₁₋₄ aliphatic optionally substituted with 1, 2, 3, 4, 5, or 6 deuterium or halogen atoms, or R⁴ and R⁵ taken together with the carbon to which they are attached form a C₃-C₈ cycloalkyl;
p is 1 or 2; and
-M- is

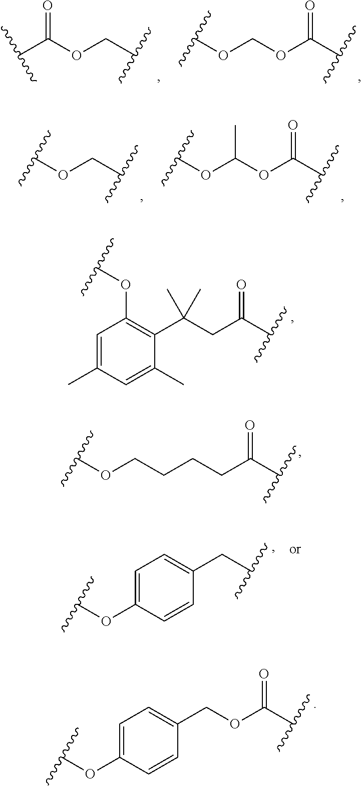

In some embodiments, each R³ is independently a saturated or unsaturated, straight C₃₋₁₇ hydrocarbon chain. In some embodiments, each R³ is a saturated C₇ hydrocarbon chain. In some embodiments, -M- is

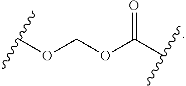 [SMILES: *OCOC(*)=O]

In some embodiments, -M- is

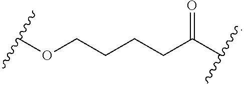 [SMILES: *OCCCCC(*)=O]

In some embodiments, R⁴ and R⁵ taken together with the carbon to which they are attached form a C₃ cycloalkyl. In some embodiments, R⁴ is methyl and R⁵ is hydrogen. In some embodiments, R⁴ and R⁵ are each methyl. In some embodiments, p is 1. In some embodiments, p is 2.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describes pharmaceutically acceptable salts in detail in J. Pharmaceutical Sciences, 1977, 66, 1-19, incorporated herein by reference.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the present disclosure are within the scope of the disclosure. Unless defined otherwise, the absolute stereochemistry for the bromolysergide (2-bromo-LSD) portion of the prodrugs described herein are as depicted. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present disclosure.

### Pharmaceutical Compositions

According to another embodiment, the present disclosure provides a pharmaceutical composition comprising a lipid prodrug of the present disclosure and a pharmaceutically acceptable carrier, adjuvant, or vehicle. The amount of lipid prodrug in the composition is an amount effective to treat the relevant disease, disorder, or condition in a patient in need thereof (an “effective amount”). In some embodiments, a composition of the present disclosure is formulated for oral administration to a patient.

The term “patient,” as used herein, means an animal, for example a mammal, such as a human.

The term “pharmaceutically acceptable carrier, adjuvant, or vehicle” refers to a non-toxic carrier, adjuvant, or vehicle that does not destroy the pharmacological activity of the agent with which it is formulated. Accordingly, the lipid prodrug of the present disclosure may be prepared as a composition with one or more pharmaceutically acceptable carriers, adjuvants, or vehicles as are known in the art. To aid in delivery, the lipid prodrug of the present disclosure may be formulated in a lipid-based formulation.

Compositions of the present disclosure may be administered orally. Lipid-based formulations for oral delivery are known in the art and may include, for example, substantially non-aqueous vehicles which typically contain one or more lipid components. The lipid vehicles and resulting lipid formulations may be usefully classified as described below according to their shared common features according to the lipid formulation classification system (LFCS) (Pouton, C. W., Eur. J. Pharm. Sci. 11 (Supp 2), S93-S98, 2000; Pouton, C. W., Eur. J. Pharm. Sci. 29 278-287, 2006).

Lipid formulations may contain lipids and/or surfactants, optionally with co-solvents, and are generally categorized into 4 types. Type I formulations include lipids which require digestion, such as mono-, di-, and triglycerides and combinations thereof. Type II formulations are water-insoluble self-emulsifying drug delivery systems (SEDDS) which contain lipids in addition to water insoluble surfactants. Type III formulations are SEDDS or self-microemulsifying drug delivery systems (SMEDDS) which contain lipids in addition to water-soluble surfactants and/or co-solvents. Type IV formulations contain predominantly hydrophilic surfactants and co-solvents such as PEG and propylene glycol.

Formulations may also contain materials commonly known to those skilled in the art to be included in lipid-based formulations, including antioxidants, for example, butylated hydroxyanisole (BHA) or butylated hydroxytoluene (BHT) and solidifying agents such as microporous silica, for example magnesium alumino-metasilicate (Neusilin®).

In some embodiments, the lipid prodrug may be co-administered orally with an enzyme inhibitor to increase stability of the prodrug in the gastrointestinal tract or enterocyte. In certain embodiments, the enzyme inhibitor inhibits pancreatic lipases, examples of which include, but are not limited to, Alli® (orlistat). In other embodiments it is envisaged that the enzyme inhibitor will inhibit cellular lipase enzymes such as monoacylglycerol lipase, an example of which includes, but is not limited to, JZL184 (4-nitrophenyl-4-[bis(1,3-benzodioxol-5-yl) (hydroxy)methyl]piperidine-1-carboxylate).

### Uses of Lymphatic-Directing Lipid Prodrugs

Disclosed herein are lymphatic-directing lipid prodrugs of bromolysergide (2-bromo-LSD) and pharmaceutically acceptable compositions thereof.

Accordingly, the present disclosure provides a method of treating a disease, disorder, or condition e.g., cluster headache, migraine, an anxiety disorder, or a mood disorder (e.g., depression (including treatment resistant depression), bipolar disorder, premenstrual syndrome dysphoric disorder (PMDD), intermittent explosive disorder (IED), or substance-induced mood disorder) in a patient comprising administering to said patient a disclosed lipid prodrug, e.g., a lipid prodrug of bromolysergide (2-bromo-LSD), or a pharmaceutically acceptable salt thereof.

The presently disclosed lipid prodrugs, e.g., lipid prodrug forms of bromolysergide (2-bromo-LSD), are useful for the stable transport of pharmaceutical agents to the intestinal lymph and release of the pharmaceutical agents in the lymph, lymphoid tissues, tissues with high lipase activity such as adipose tissue, the liver, or in the systemic circulation.

In some embodiments, the present disclosure provides a method of improving the pharmacokinetic profile of bromolysergide (2-bromo-LSD). In some embodiments, the present disclosure provides a method of administering bromolysergide (2-bromo-LSD), wherein the bioavailability of bromolysergide (2-bromo-LSD) is improved, the method comprising administering to a patient in need thereof an effective amount of a disclosed lipid prodrug form bromolysergide (2-bromo-LSD). In some embodiments, the compositions described herein comprising a lipid prodrug form of bromolysergide (2-bromo-LSD) are useful for improving the pharmacokinetic profile of bromolysergide (2-bromo-LSD) upon administration to the subject.

In some embodiments, the present disclosure provides a method of improving the therapeutic index (i.e., the range of doses at which a therapeutic agent is effective without unacceptable adverse events) of bromolysergide (2-bromo-LSD). In some embodiments, the present disclosure provides a method of administering bromolysergide (2-bromo-LSD), wherein the therapeutic index of bromolysergide (2-bromo-LSD) is improved, the method comprising administering to a patient in need thereof an effective amount of a disclosed lipid prodrug form of bromolysergide (2-bromo-LSD). In some embodiments, the compositions described herein comprising a lipid prodrug form of bromolysergide (2-bromo-LSD) are useful for improving the therapeutic index of bromolysergide (2-bromo-LSD) upon administration.

In some embodiments, the disclosure provides a method for treating, reducing, ameliorating, or eliminating one or more symptom(s) associated with cluster headaches and/or migraines in a patient comprising administering to the patient the prodrug or compound of formula (I), (II), (III), (IV), (V), or (VI) or a pharmaceutically acceptable composition comprising the prodrug or compound of formula (I), (II), (III), (IV), (V), or (VI). In some embodiments, the disclosure provides a method for treating, reducing, ameliorating, or eliminating one or more symptom(s) associated with a mood or anxiety disorder in a patient comprising administering to the patient the compound of formula (I), (II), (III), (IV), (V), or (VI) or a pharmaceutically acceptable composition comprising the compound of formula (I), (II), (III), (IV), (V), or (VI). In some embodiments, the mood disorder is depression, bipolar disorder, premenstrual syndrome dysphoric disorder (PMDD), intermittent explosive disorder (IED), or substance-induced mood disorder. In some further embodiments, mood disorder includes depressive disorders and/or anxiety disorders. In some embodiments, the depressive disorder is major depressive disorder (MDD), persistent depressive disorder (PDD), bipolar disorder, seasonal affective disorder (SAD), and/or post-traumatic stress disorder (PTSD). In some embodiments, the depressive disorder is treatment resistant depression (TRD). In some embodiments, the anxiety disorder is generalized anxiety disorder, social anxiety disorder, separation anxiety disorder, or one or more phobias. In some embodiments, the prodrug is selected from Table 1.

### Combination Therapies

The provided lipid prodrug, or pharmaceutically acceptable composition thereof, may be administered to a patient in need thereof in combination with one or more additional therapeutic agents and/or therapeutic processes.

The lipid prodrug or pharmaceutically acceptable composition thereof can be administered alone or in combination with one or more other therapeutic compounds, possible combination therapy taking the form of fixed combinations, or the administration of the lipid prodrug or composition and one or more other therapeutic compounds being staggered or given independently of one another, or the combined administration of fixed combinations and one or more other therapeutic compounds. Long-term therapy is equally possible as is adjuvant therapy in the context of other treatment strategies, as described above.

Such additional agents may be administered separately from a provided lipid prodrug or composition, as part of a multiple dosage regimen. Alternatively, those agents may be part of a single dosage form, mixed together with a disclosed lipid prodrug in a single composition. If administered as part of a multiple dosage regime, the two active agents may be submitted simultaneously, sequentially or within a period of time from one another.

As used herein, the term “combination,” “combined,” and related terms refers to the simultaneous or sequential administration of therapeutic agents in accordance with the present disclosure. For example, a disclosed lipid prodrug may be administered with one or more other therapeutic agent(s) simultaneously or sequentially in separate unit dosage forms or together in a single unit dosage form. Accordingly, the present disclosure provides a single unit dosage form comprising a disclosed lipid prodrug, one or more additional therapeutic agent(s), and a pharmaceutically acceptable carrier, adjuvant, or vehicle. In some embodiments, the additional agent is formulated in a separate composition from the lipid prodrug. In some embodiments, the one or more additional agents are lipid prodrugs, e.g., as described herein or made according to methods described herein.

The amount of additional therapeutic agent present in the compositions of this disclosure will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

Examples of agents with which the lipid prodrugs of this disclosure may be combined include, without limitation: treatments for cluster headaches and/or migraines such as oxygen, triptans (e.g., sumatriptan and zolmitriptan), octreotide, local anesthetics (e.g., lidocaine), dihydroergotamine, calcium channel blocking agents (e.g., verapamil), corticosteroids (e.g., prednisone), anti-CGRP antibodies (e.g., galcanezumab), lithium carbonate (LITHOBID®), anti-seizure medicines (e.g., topiramate), caffeine, ergotamine, melatonin, capsaicin, zucapsaicin, and/or sodium valproate; and agents for treating mood disorders (e.g., depression (including treatment resistant depression), bipolar disorder, premenstrual syndrome dysphoric disorder (PMDD), intermittent explosive disorder (IED), or substance-induced mood disorder) such as sodium valproate, varbamazepine, oxcarbazepine, lamotrigine, benzodiazepines (e.g., alprazolam, chlordiazepoxide, clobazam, clonazepam, clorazepate, diazepam, estazolam, flurazepam, lorazepam, midazolam, oxazepam, quazepam, remimazolam, temazepam, and triazolam), triiodothyronine (thyroid hormone), selective serotonin reuptake inhibitors (SSRIs) (e.g., sertraline, fluvoxamine, fluoxetine, citalopram, escitalopram, and paroxetine), serotonin-norepinephrine reuptake inhibitors (SNRIs) (e.g., venlafaxine, desvenlafaxine, duloxetine, milnacipran, and levomilnacipran, atypical antidepressants (e.g., bupropion, mirtazapine), tricyclic antidepressants (TCAs) (e.g., desipramine, nortriptyline, imipramine, and amitriptyline), serotonin modulators (e.g., nefazodone, trazodone, vilazodone, and vortioxetine), monoamine oxidase inhibitors (MAOIs) (e.g., tranylcypromine, phenelzine, and selegiline), and aripiprazole. In some embodiments, the additional therapeutic agent is a serotonin receptor agonist.

### General Methods for Making Lipid Prodrugs

The lipid prodrug compounds of this invention may be prepared or isolated in general by synthetic and/or semi-synthetic methods known to those skilled in the art for analogous compounds and by methods described in detail in the Examples, herein.

The therapeutic agents comprised in disclosed lipid prodrugs (e.g., conjugated to a glyceride-based prodrug) may be purchased commercially or prepared by organic synthesis, semi-synthesis, and like methods known in the art.

In some embodiments, protecting groups (as defined below) can be used to manipulate therapeutic agents in preparation for conjugation to the remainder of the lipid prodrug structure, for example, to prevent undesired side reactions from taking place.

In order that the invention described herein may be more fully understood, the following examples are set forth. It should be understood that these examples are for illustrative purposes only and are not to be construed as limiting this invention in any manner.

### EXAMPLES

The following examples describe the synthesis of prodrugs, including intermediates, for use in the methods disclosed herein. As used throughout, “eq” refers to molar equivalents. Unless otherwise specified, intermediate numbering lacking any prefix (e.g., 1, 2, 3, etc.) is particular to each scheme (or structural drawing) in which it appears, while intermediate numbering including an “Int” prefix (e.g., Int-1, Int-2, Int-3, etc.) is used to refer to the same intermediate across schemes (or structural drawings).

As used throughout, 2BL refers to bromolysergide (2-Bromo-LSD):

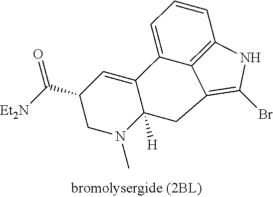

### Example 1: Acid Intermediates

### Synthesis of Int-1 (C5bMe-acid-2-TG-oleate)

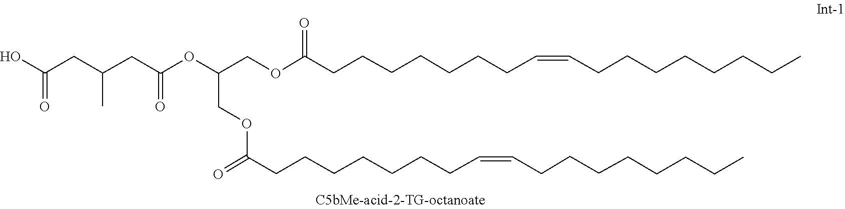 [SMILES: CCCCCCCC/C=C\CCCCCCCC(=O)OCC(COC(=O)CCCCCCC/C=C\CCCCCCCC)OC(=O)CC(C)CC(=O)O]

The synthesis of Int-1 is described in WO2021/159021 (see paragraphs [00335]-[00336]).

### Synthesis of Int-2 (C5bMe-acid-2-TG-octanoate)

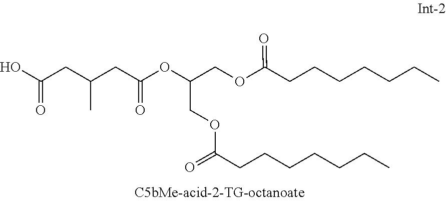 [SMILES: CCCCCCCC(=O)OCC(COC(=O)CCCCCCC)OC(=O)CC(C)CC(=O)O]

The synthesis of Int-2 is described in WO2021/159021 (see paragraphs [00761]-[00762]).

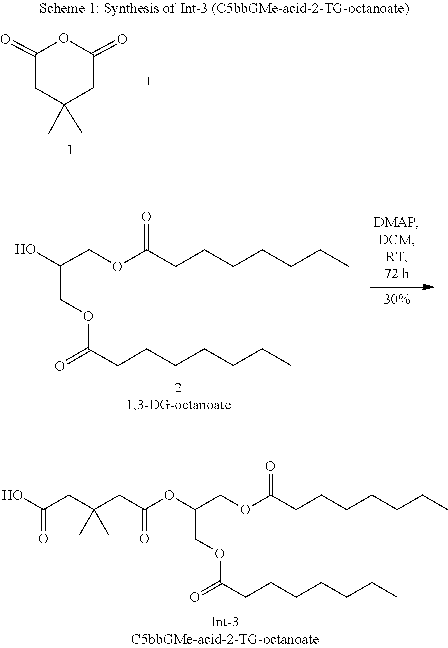

4-Dimethylaminopyridine (DMAP, 1.19 g, 9.71 mmol) was added to a solution of 3,3-dimethylglutaric anhydride 1 (1.38 g, 9.71 mmol) and 1,3-DG-octanoate 2 (2.23 g, 6.47 mmol, see paragraphs [00545]-[00546] of WO2021/159021) in dichloromethane (DCM, 40 mL). The mixture was stirred for 3 days at room temperature (RT), then concentrated to afford crude Int-3 as an oil. The crude product was purified by normal phase purification (Biotage Isolera, 120 g, SiliaSep cartridge) using a 0-20% ethyl acetate in heptane eluent over 14 column volumes (CVs) to afford pure Int-3 (951 mg, 30.2% yield) as a colorless oil.

¹H NMR (400 MHz, CDCl₃): δ 5.31-5.25 (m, 1H), 4.29 (dd, J=4.2, 12.0 Hz, 2H), 4.14 (dd, J=6.1, 12.0 Hz, 2H), 2.47 (s, 2H), 2.46 (s, 2H), 2.30 (t, J=7.6 Hz, 4H), 1.65-1.54 (m, 4H), 1.35-1.21 (m, 16H), 1.14 (s, 6H), 0.89-0.84 (m, 6H); exchangeable CO₂H proton not observed. By ¹H NMR, compound contained 20.2% (w/w %) 3,3-dimethylglutaric anhydride and 1.7% (w/w) DCM.

UPLC-MS: (XB BEH300 C4 20-95%): Rₜ=3.58 minutes, 97.3% (UV), 100.0% (ELSD). MS (ESIpos): m/z=504.7 [M+NH₄]⁺; MS (ESIneg): m/z=485.7 [M−H]⁻

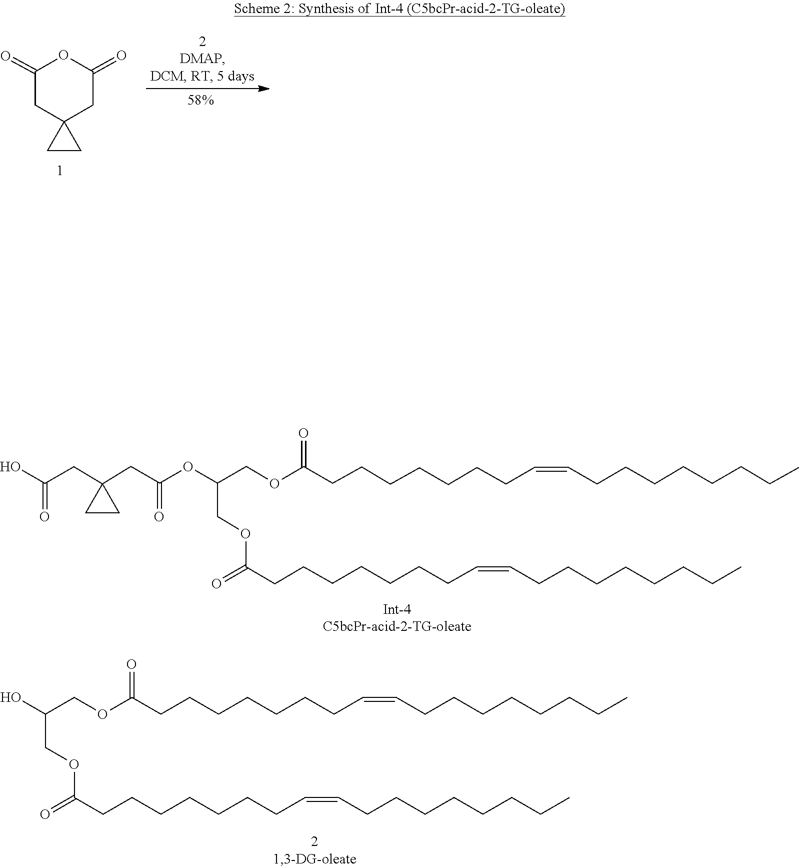 [SMILES: O=C1CC2(CC2)CC(=O)O1 ; CCCCCCCC/C=C\CCCCCCCC(=O)OCC(COC(=O)CCCCCCC/C=C\CCCCCCCC)OC(=O)CC1(CC(=O)O)CC1 ; CCCCCCCC/C=C\CCCCCCCC(=O)OCC(O)COC(=O)CCCCCCC/C=C\CCCCCCCC]

DMAP (1.50 g, 12.3 mmol) was added to a solution of 6-oxaspiro[2.5]octane-5,7-dione 1 (1.69 g, 12.1 mmol) and 1,3-DG-oleate 2 (5.00 g, 8.05 mmol, see paragraphs [00526]-[00528] of WO2021/159021) in DCM (60 mL). The mixture was stirred at RT for 5 days and then concentrated to afford crude Int-4. Crude product was purified by normal phase purification (Biotage Isolera, 120 g SiliaSep cartridge) using a 40-60% ethyl acetate in heptane eluent over 15 CVs to afford pure Int-4 (3.57 g, 58.3% yield) as a colorless oil.

¹H NMR (400 MHZ, CDCl₃): δ 5.46-5.22 (m, 5H), 4.31 (dd, J=4.3, 11.9 Hz, 2H), 4.18-4.11 (m, 2H), 2.46 (s, 2H), 2.45 (s, 2H), 2.31 (t, J=7.5 Hz, 4H), 2.07-1.97 (m, 8H), 1.68-1.56 (m, 4H), 1.37-1.22 (m, 40H), 0.92-0.83 (m, 6H), 0.57 (s, 4H). 80H expected 79H observed, exchangeable CO₂H proton not observed.

¹³C NMR (100 MHZ, CDCl₃): δ 177.2, 173.4, 171.4, 130.2, 129.9, 69.3, 62.2, 41.1, 40.8, 34.2, 32.1, 29.91, 29.85, 29.7, 29.5, 29.31, 29.26, 29.24, 27.4, 27.3, 25.0, 22.8, 14.3, 14.2, 12.4.

UPLC-MS (XB BEH300 C4 20 to 95%): Rₜ=6.32 minutes, 100.0% (UV), 99.9% (ELSD); MS(ESIpos): m/z=779.1 [M+H]⁺, MS(ESIneg): m/z=760.1 [M−H]⁻.

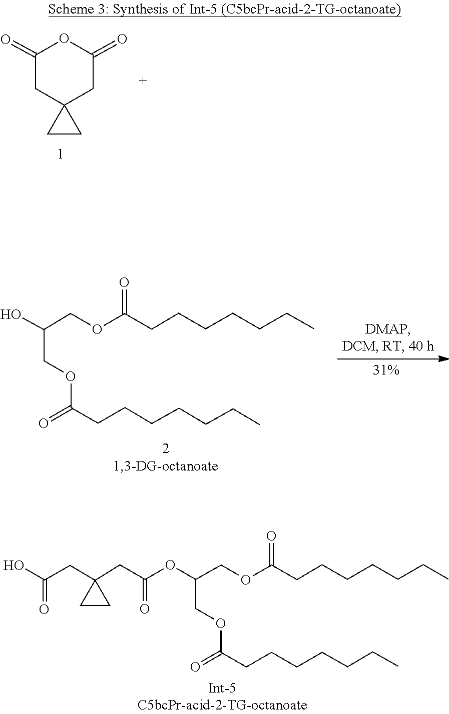 [SMILES: O=C1CC2(CC2)CC(=O)O1 ; CCCCCCCC(=O)OCC(O)COC(=O)CCCCCCC ; CCCCCCCC(=O)OCC(COC(=O)CCCCCCC)OC(=O)CC1(CC(=O)O)CC1]

DMAP (1.36 g, 11.1 mmol) was added to a solution of 6-oxaspiro[2.5]octane-5,7-dione 1 (1.53 g, 11.0 mmol) and 1,3-DG-octanoate 2 (2.51 g, 7.30 mmol) in DCM (50 mL). The mixture was stirred at RT for 40 hours and concentrated to afford crude Int-5. Crude product was purified by normal phase purification (Biotage Isolera, 120 g SiliaSep cartridge), using a 0-40% ethyl acetate in heptane eluent over 12 CVs to afford pure Int-5 (1.10 g, 31.1% yield) as a colorless oil.

¹H NMR (400 MHZ, CDCl₃): δ 5.27 (quin, J=5.0 Hz, 1H), 4.37-4.25 (m, 2H), 4.14 (dd, J=5.9, 11.9 Hz, 2H), 2.51-2.40 (m, 4H), 2.31 (t, J=7.5 Hz, 4H), 1.67-1.55 (m, 4H), 1.34-1.21 (m, 16H), 0.90-0.83 (m, 6H), 0.57 (s, 4H). Expected 44H, observed 43H. Exchangeable CO₂H proton not observed.

¹³C NMR (101 MHz, CDCl₃): δ 173.5, 171.5, 171.4, 69.3, 62.2, 41.0, 40.9, 40.8, 34.2, 31.8, 29.2, 29.0, 25.0, 22.7, 14.2, 12.4.

UPLC-MS: (XB BEH 300 C4 20-95%): Rt=3.35 minutes, 92.4% (UV), 100.0% (ELSD); MS(ESIpos): m/z=502.7 [M+H]⁺.

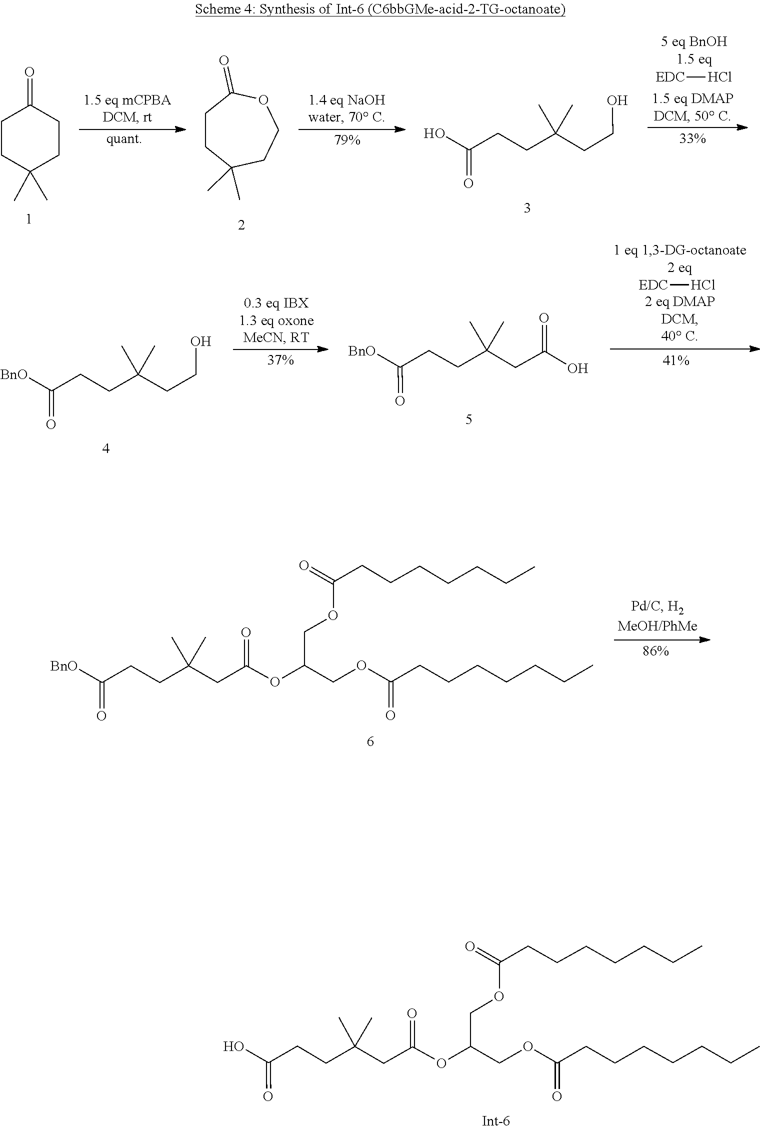

To a solution of 4,4-dimethylcyclohexanone 1 (2.26 g, 17.9 mmol) in DCM (50 mL), 3-chloroperoxybenzoic acid (mCPBA, 4.64 g, 26.9 mmol) was added at RT. The reaction was stirred at RT for 16 hours. The white solid which formed was filtered off and washed with DCM (3×50 mL). The filtrated was washed with aqueous NaHCO₃ (3×100 mL). The organic phase was dried over MgSO₄, filtered, and concentrated to afford 2 (2.60 g, crude) as a white solid. The product was used in the next step without further purification.

¹H NMR (400 MHz, CDCl₃): δ 4.21-4.19 (m, 2H), 2.60 (t, J=6.0 Hz, 2H), 1.64 (t, J=4.8 Hz, 2H), 1.56 (t, J=6.0 Hz, 2H), 1.02 (s, 6H).

To a solution of 5,5-dimethyloxepan-2-one 2 (1.50 g, 10.6 mmol) in water (10 mL) at RT, sodium hydroxide (612 mg, 15.3 mmol) was added in one portion. The reaction mixture stirred at 70° C. for 16 hours. The reaction mixture was cooled to RT and the pH was adjusted to pH=3 with 2N HCl. The organics were extracted with diethyl ether (3×50 mL). The combined organics were washed with brine (100 mL), dried over MgSO₄, filtered, and concentrated to afford 3 (1.35 g, crude) as a colorless gum. The product was used in the next step without further purification.

¹H NMR (400 MHZ, CDCl₃): δ 6.70-6.30 (br s, 1H), 3.71 (t, J=8.8 Hz, 2H), 2.32 (t, J=7.4 Hz, 2H), 1.59 (t, J=8.2 Hz, 2H), 1.52 (t, J=7.6 Hz, 2H), 0.94 (s, 6H); Exchangeable CO₂H proton not observed.

¹³C NMR (100 MHz, CDCl₃): δ 179.7, 133.7, 130.3, 129.9, 128.3, 65.0, 59.6, 43.9, 43.8, 42.0, 36.8, 35.8, 32.1, 32.0, 30.2, 29.5, 27.2.

To a solution of 6-hydroxy-4,4-dimethyl-hexanoic acid 3 (1.35 g, 8.43 mmol) in DCM (150 mL), DMAP (1.54 g, 12.6 mmol), EDC-HCl (2.42 g, 12.6 mmol) and benzyl alcohol (9.11 g, 84.3 mmol, 8.72 mL) were added and the mixture was heated to 50° C. for 16 hours. The reaction mixture was cooled to RT, and aqueous 2N HCl (50 mL) was added. The organics were extracted with ethyl acetate (3×50 mL). The combined organic layers were washed with brine (100 mL), dried over MgSO₄, filtered, and concentrated to afford a crude residue. The material was purified by normal phase chromatography (Biotage Isolera, 120 g SiliCycle cartridge; eluent 0-100% ethyl acetate in heptane over 10 CVs) to afford 4 (721 mg, 33% yield) as a colorless oil.

¹H NMR (400 MHZ, CDCl₃): δ 7.39-7.30 (m, 5H), 5.11 (s, 2H), 3.69 (t, J=8 Hz, 2H), 2.36-2.32 (m, 2H), 1.64-1.57 (m, 2H), 1.56-1.46 (m, 2H), 1.30 (s, 1H), 0.90 (s, 6H).

¹³C NMR (100 MHZ, CDCl₃): δ 174.2, 136.1, 128.7, 128.4, 66.4, 59.7, 44.2, 37.0, 32.1, 29.7, 27.2.

To a solution of benzyl 6-hydroxy-4,4-dimethyl-hexanoate 4 (721 mg, 2.88 mmol) in acetonitrile (2 mL), oxone (2.30 g, 3.74 mmol) and IBX (242 mg, 864 μmol) were added. The reaction mixture was heated at 70° C. and stirred for 4 hours. The reaction mixture was cooled to RT, and an aqueous 2N HCl solution (20 mL) was added. The mixture was extracted with ethyl acetate (3×50 mL). The combined organic layers were washed with brine (50 mL), dried over MgSO₄, filtered and concentrated. The material was purified by normal phase chromatography (Biotage Isolera, 25 g SiliCycle cartridge; eluent 0-100% ethyl acetate in heptane over 15 CVs) to afford 5 (384 mg, 38% yield) as a colorless oil.

¹H NMR (400 MHZ, CDCl₃): δ 7.40-7.30 (m, 5H), 5.11 (s, 2H), 2.40-2.36 (m, 2H), 2.23 (s, 2H), 1.77-1.72 (m, 2H), 1.03 (s, 6H); exchangeable CO₂H proton not observed.

C_UPLC2-MS: (CSH-C18 Long Neutral 2 to 95%): Rₜ=2.22 min (75.2%), MS (ESIneg): m/z=[M−H]⁻ 263.2.

To a solution of 6-benzyloxy-3,3-dimethyl-6-oxo-hexanoic acid 5 (384 mg, 1.45 mmol) and 1,3-DG-octanoate (500 mg, 1.45 mmol) in DCM (10 mL), EDC-HCl (557 mg, 2.91 mmol) and DMAP (355 mg, 2.91 mmol) were added. The reaction was stirred at 30° C. for 16 hours. The reaction mixture was cooled to RT and quenched with a saturated aqueous solution of NH₄Cl (50 mL). The mixture was extracted with ethyl acetate (3×20 mL). The combined organic layers were washed with brine (100 mL), dried over MgSO₄, filtered, and concentrated to give a crude residue. The material was purified by normal phase chromatography (Biotage Isolera, 40 g SiliCycle cartridge; eluent 0-40% ethyl acetate in heptane over 15 CVs) to afford 6 (446 mg, 41% yield) as colorless oil.

UPLC4-MS (XB BEH300 C4 20 to95%): Rₜ=5.56 min., 71.6% (UV), 79.3% (ELSD); MS(ESIpos): m/z=608.8 [M+NH₄]⁺.

¹H NMR (400 MHZ, CDCl₃): δ 7.38-7.30 (m, 5H), 5.29-5.24 (m, 1H), 5.11 (s, 2H), 4.28 (dd, J=4.2, 11.9 Hz, 2H), 4.12 (dd, J=6.1, 11.9 Hz, 2H), 2.39-2.35 (m, 2H), 2.29 (t, J=7.6 Hz, 4H), 2.22 (s, 2H), 1.73-1.71 (m, 2H), 1.61-1.58 (m, 4H), 1.28-1.27 (m, 16H), 1.00 (s, 6H), 0.93-0.86 (m, 6H).

To a solution of 6-benzyl 1-(1,3-bis(octanoyloxy) propan-2-yl) 3,3-dimethylhexanedioate 6 (446 mg, 755 μmol) in methanol (20 mL) under argon, palladium on carbon (5% w/w) (803 mg, 377 μmol) was added. A hydrogen atmosphere was introduced, and the reaction mixture was stirred at RT for 16 hours. The reaction mixture was filtered over celite, and the organics were concentrated to give a yellow oil. The crude oil was passed through a silica plug to afford Int-6 (325 mg, crude).

UPLC4-MS (XB BEH300 C4 20 to95%): Rₜ=3.90 min., 55.2% (UV), 98.9% (ELSD); MS(ESIpos): m/z=501.7 [M+H]⁺, 518.7 [M+NH₄]⁺.

¹H NMR (400 MHZ, CDCl₃): δ 5.30-5.25 (m, 1H), 4.28 (dd, J=4.2, 11.9 Hz, 2H), 4.13 (dd, J=6.1, 11.9 Hz, 2H), 2.38-2.35 (m, 2H), 2.30 (t, J=8 Hz, 4H), 2.23 (s, 2H), 1.72-1.68 (m, 2H), 1.60 (br t, J=7.2 Hz, 4H), 1.28 (br s, 16H), 1.02 (s, 6H), 0.89-0.86 (m, 6H); exchangeable CO₂H proton not observed.

¹³C NMR (100 MHZ, CDCl₃): δ 179.7, 173.6, 171.1, 69.0, 62.3, 45.7, 37.1, 34.2, 33.2, 31.8, 30.4, 29.2, 29.0, 27.2, 25.0, 22.7, 14.2.

### Synthesis of Int-16 (C8bMe-acid-2-TG-oleate)

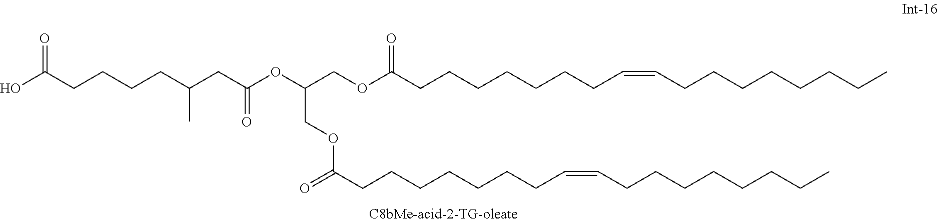 [SMILES: CCCCCCCC/C=C\CCCCCCCC(=O)OCC(COC(=O)CCCCCCC/C=C\CCCCCCCC)OC(=O)CC(C)CCCCC(=O)O]

The synthesis of Int-16 is described in WO2021/159021 (see paragraphs [00397]-[00399]).

### Synthesis of Int-17 (C12b′bMe-acid-2-TG-oleate)

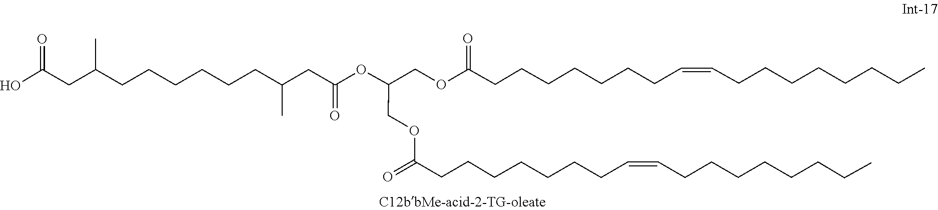 [SMILES: CCCCCCCC/C=C\CCCCCCCC(=O)OCC(COC(=O)CCCCCCC/C=C\CCCCCCCC)OC(=O)CC(C)CCCCCCC(C)CC(=O)O]

The synthesis of Int-17 is described in WO2021/159021 (see paragraphs [00633]-[00636]).

### Synthesis of Int-18 (C12bMe-acid-2-TG-oleate)

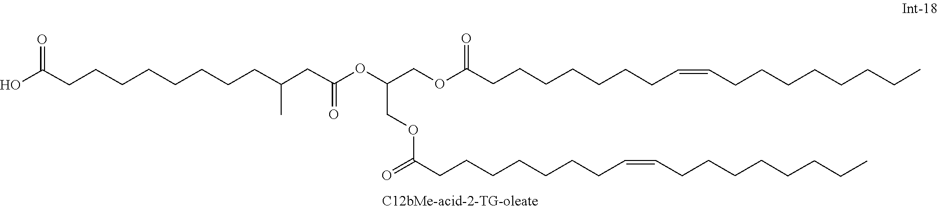 [SMILES: CCCCCCCC/C=C\CCCCCCCC(=O)OCC(COC(=O)CCCCCCC/C=C\CCCCCCCC)OC(=O)CC(C)CCCCCCCCC(=O)O]

The synthesis of Int-18 is described in WO2021/159021 (see paragraphs [00409]-[00413]).

### Synthesis of Int-19 (C10b′bMe-acid-2-TG-oleate)

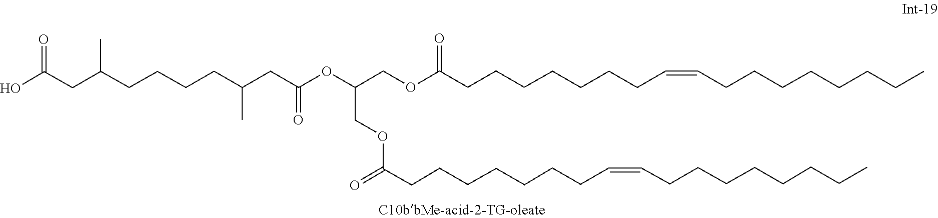 [SMILES: CCCCCCCC/C=C\CCCCCCCC(=O)OCC(COC(=O)CCCCCCC/C=C\CCCCCCCC)OC(=O)CC(C)CCCCC(C)CC(=O)O]

The synthesis of Int-19 is described in WO2021/159021 (see paragraphs [00626]-[00632]).

### Example 2: Synthesis of Bromolysergide (2-Bromo-LSD) Lipid Prodrugs

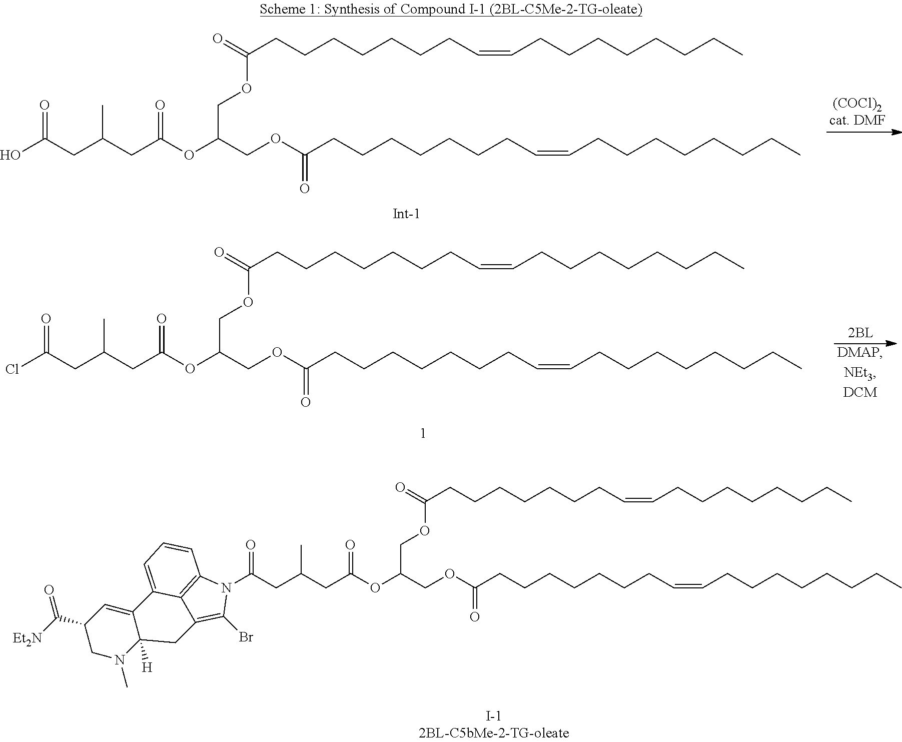

To a solution of Int-1 (500 mg, 667 μmol) in DCM (4 mL) and N,N-dimethylformamide (DMF, 2 drops) at 0° C. was added a solution of oxalyl chloride (436 mg, 3.44 mmol, 300 μL) in DCM (10 mL) dropwise and the mixture was stirred at RT for 18 hours. The mixture was concentrated to afford 1 (645 mg, 78.4% yield) as an orange oil.

¹H NMR (400 MHZ, CDCl₃): δ 5.36-5.26 (m, 5H), 4.33 (dd, J=11.9, 4.1 Hz, 2H), 4.13 (ddd, J=12.0, 6.1, 1.1 Hz, 2H), 3.11-2.73 (m, 2H), 2.60-2.48 (m, 1H), 2.44-2.27 (m, 6H), 2.08-1.96 (m, 8H), 1.65-1.56 (m, 4H), 1.38-1.23 (m, 40H), 1.08 (d, J=6.4 Hz, 3H), 0.93-0.85 (m, 6H).

To a stirring solution of 2BL (20.0 mg, 49.7 μmol) in DCM (1.5 mL) was added DMAP (2.00 mg, 16.4 μmol) and triethylamine (8.71 mg, 86.1 μmol, 12.0 μL). The mixture was cooled to 0° C. and a solution of 1 (35.0 mg, 45.6 μmol) in DCM (0.5 mL) was added and the mixture was stirred at room temperature for 18 hours. The mixture was quenched with saturated aqueous NH₄Cl (10 mL) and diluted with ethyl acetate (10 mL). The layers were separated, and the organic layer was washed with brine (10 mL), dried over MgSO₄, filtered and concentrated in vacuo. The material was purified by normal phase chromatography (Biotage Isolera, 4 g Siliasep cartridge; eluent 50-100% ethyl acetate in heptane [6CVs 50%, 20 CVs 50-100%]) to afford Compound I-1 (3 mg, 5.3% yield) as a yellow oil.

UPLC4-MS: (XB BEH 300 C4 20-95%): Rₜ=7.20 min., 40.5% (UV), 74.3% (ELSD); MS(ESIpos): m/z=1133.5/1135.4 [M+H]+; 79Br/81Br isotope pattern observed.

¹H NMR (400 MHZ, CDCl₃): δ 7.99 (br d, J=7.8 Hz, 1H), 7.39-7.34 (m, 1H), 7.33-7.27 (m, 1H), 6.39 (s, 1H), 5.40-5.23 (m, 5H), 4.30 (ddd, J=1.6, 4.4, 11.9 Hz, 2H), 4.21-4.02 (m, 2H), 3.57-3.36 (m, 5H), 3.33-3.22 (m, 2H), 3.20-3.08 (m, 2H), 2.81-2.69 (m, 2H), 2.62-2.51 (m, 1H), 2.46-2.36 (m, 1H), 2.29 (t, J=7.6 Hz, 4H), 2.08-1.94 (m, 8H), 1.65-1.52 (br s, 9H), 1.37-1.23 (m, 43H), 1.18 (t, J=7.1 Hz, 3H), 1.13 (d, J=6.9 Hz, 3H), 0.90-0.85 (m, 6H).

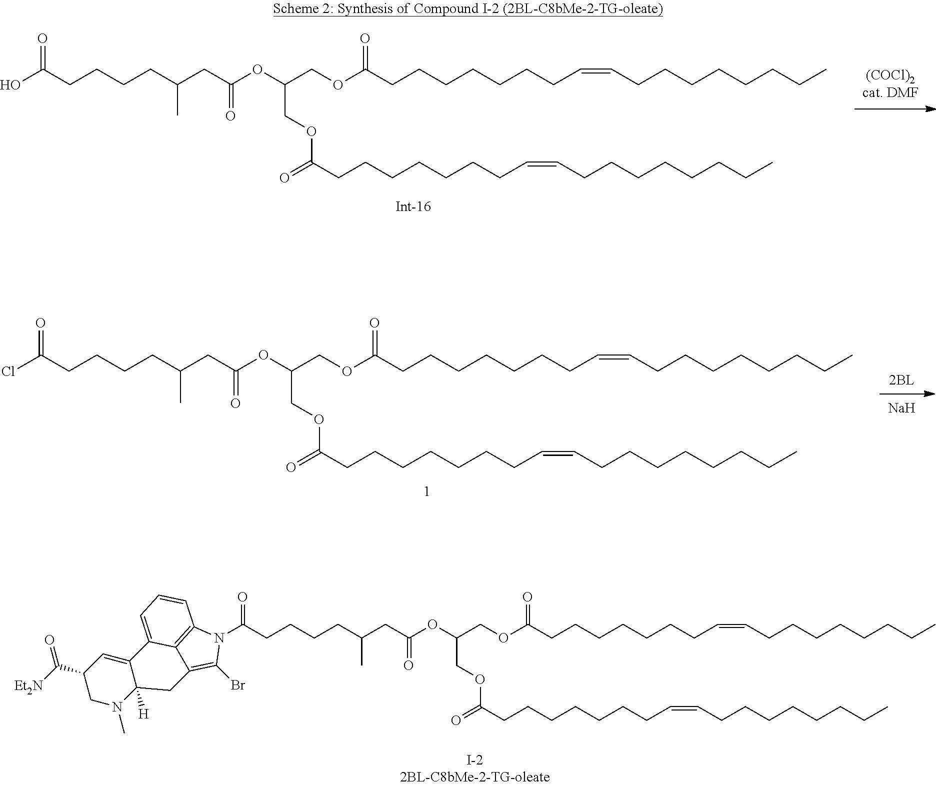

To a solution of Int-16 (50.0 mg, 64.3 μmol) in a mixture of DMF (10 μL, 129 μmol) and dichloromethane (2 mL) was added oxalyl chloride (6.74 μL, 77.2 μmol) and the reaction was stirred at room temperature for 2 hours. The reaction mixture was concentrated to afford 1 (51.2 mg, quantitative) as a light yellow oil. Acid chloride 1 was used directly in the next reaction.

A suspension of 2BL (12.0 mg, 29.8 μmol) and sodium hydride (2.15 mg, 89.5 μmol, 60% dispersion in mineral oil) in DMF (1 mL) was heated to 50° C. and stirred for 30 minutes. To the solution was added a solution of 1 (48.3 mg, 59.7 μmol) in DMF (1 mL) over 5 minutes, and the mixture was stirred at 50° C. for 18 hours. The reaction mixture was cooled to room temperature, diluted with diethyl ether (10 mL) and washed with water (2×10 mL). The organic phase was dried over MgSO₄, filtered and concentrated. The material was purified by normal phase chromatography (Biotage Isolera, 4 g SiliaSep cartridge; eluent 0-60% ethyl acetate in dichloromethane over 50 CV) to afford Compound I-2 (17.0 mg, 48% yield) as a dark yellow oil.

UPLC4-MS (XB BEH300 C4 20 to 95%): Rₜ=7.30 min., 79.0% (UV), 99.0% (ELSD); MS (ESIpos): m/z=1175.3, 1177.3 [M+H]+. 79Br/81Br isotope pair observed.

¹H NMR (400 MHZ, CDCl₃) δ=7.96 (d, J=8.2 Hz, 1H), 7.36 (d, J=7.3 Hz, 1H), 7.28 (d, J=8.0 Hz, 1H), 6.37 (s, 1H), 5.38-5.26 (m, 5H), 4.29 (dd, J=4.2, 11.9 Hz, 2H), 4.14 (dd, J=6.0, 11.9 Hz, 2H), 4.04-3.83 (m, 1H), 3.49-3.41 (m, 4H), 3.39-3.05 (m, 4H), 2.96-2.85 (br s, 1H), 2.70-2.43 (m, 3H), 2.37-2.28 (m, 5H), 2.14 (dd, J=8.3, 14.7 Hz, 1H), 2.07-1.91 (m, 9H), 1.85-1.79 (m, 2H), 1.65-1.55 (m, 4H), 1.52-1.37 (m, 2H), 1.36-1.20 (m, 47H), 1.17 (t, J=7.1 Hz, 3H), 0.95 (d, J=6.6 Hz, 3H), 0.87 (t, J=6.8 Hz, 6H).

¹³C NMR (100 MHZ, CDCl₃): δ 173.4, 172.8, 172.3, 135.6, 130.2, 129.9, 126.4, 121.6, 117.6, 115.5, 69.0, 62.3, 62.0, 55.8, 43.8, 42.3, 41.7, 40.5, 39.1, 36.6, 34.2, 32.0, 30.3, 29.91, 29.85, 29.80, 29.7, 29.50, 29.46, 29.31, 29.26, 29.23, 27.4, 27.3, 26.6, 25.2, 25.0, 22.8, 19.6, 15.1, 14.3, 13.3.

### Synthesis of Compound I-3 (2BL-C12b′bMe-2-TG-oleate)

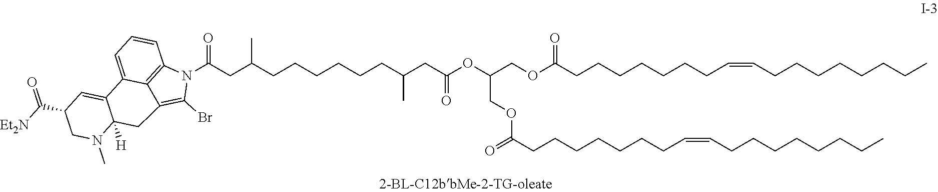

Compound I-3 is prepared similarly to Compound I-2, as described above, from intermediate C12b′bMe-acid-2-TG-oleate (Int-17), prepared as described in Example 1.

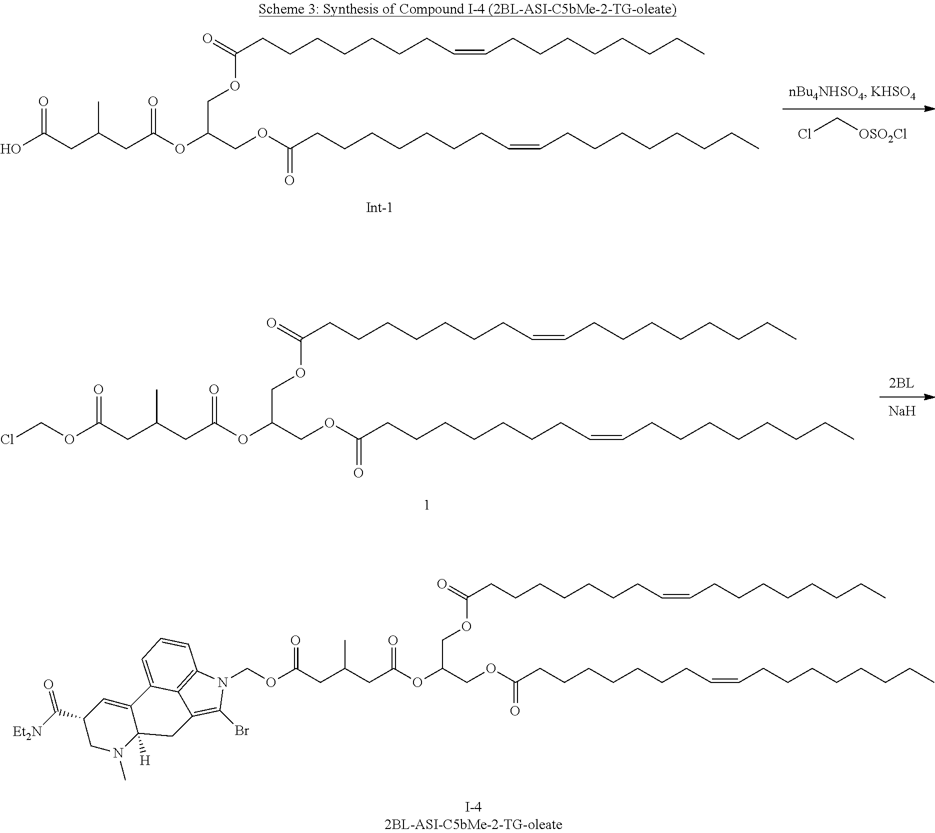

Tetra-n-butylammonium hydrogen sulfate (0.4 eq) and potassium hydrogen sulfate (4 eq) in water (0.2 M relative to KHSO₄) are added to a solution of Int-1 (1 eq) in DCM (0.05 M) and the reaction is stirred at RT for about 30 minutes. Chloromethyl chlorosulfate (1.2 eq) is added dropwise and the reaction is stirred vigorously at RT for about 16 hours. The reaction is diluted with DCM, washed with water and brine, dried with MgSO₄, and concentrated under reduced pressure to provide the crude reaction product. Purification by silica gel chromatography with a suitable solvent mixture affords 1.

A solution of 2BL (1 eq) in THF (0.1 M) is added to a suspension of NaH (60% w/w dispersion in mineral oil, 1 eq) in THF (0.1 M) at 0° C. and the reaction is stirred at 0° C. for around 30 minutes. A solution of 1 (1.2 eq) in THF (0.1 M) is slowly added to the stirred reaction. The mixture is then stirred at 0° C. for 30 minutes, allowed to warm to RT, and further stirred at RT for 4-24 hours. The reaction is quenched by the slow addition of saturated aqueous NH₄Cl, diluted with water, and extracted with ethyl acetate. The combined organic extracts are washed with water and brine, dried with MgSO₄, and concentrated under reduced pressure to give the crude reaction product. Purification by silica gel chromatography with a suitable solvent mixture affords 2BL-ASI-C5bMe-2-TG-oleate, Compound I-4.

### Synthesis of Compound I-5 (2BL-ASI-C8bMe-2-TG-oleate)

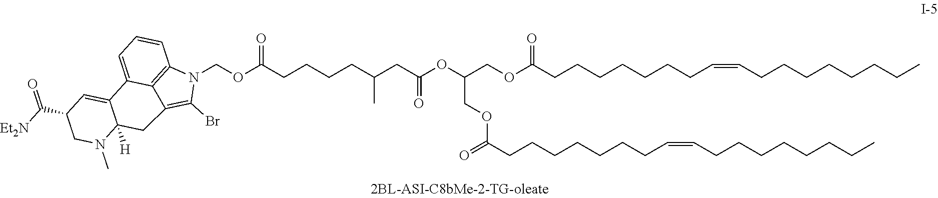

Compound I-5 is prepared similarly to Compound I-4, as described above, from intermediate C8bMe-acid-2-TG-oleate (Int-16), prepared as described in Example 1. Synthesis of Compound I-6 (2BL-ASI-C12bMe-2-TG-oleate)

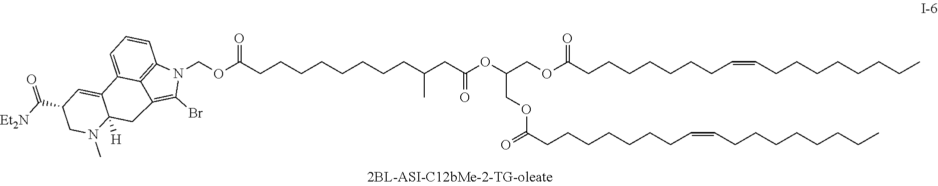

Compound I-6 is prepared similarly to Compound I-4, as described above, from intermediate C12bMe-acid-2-TG-oleate (Int-18), prepared as described in Example 1.

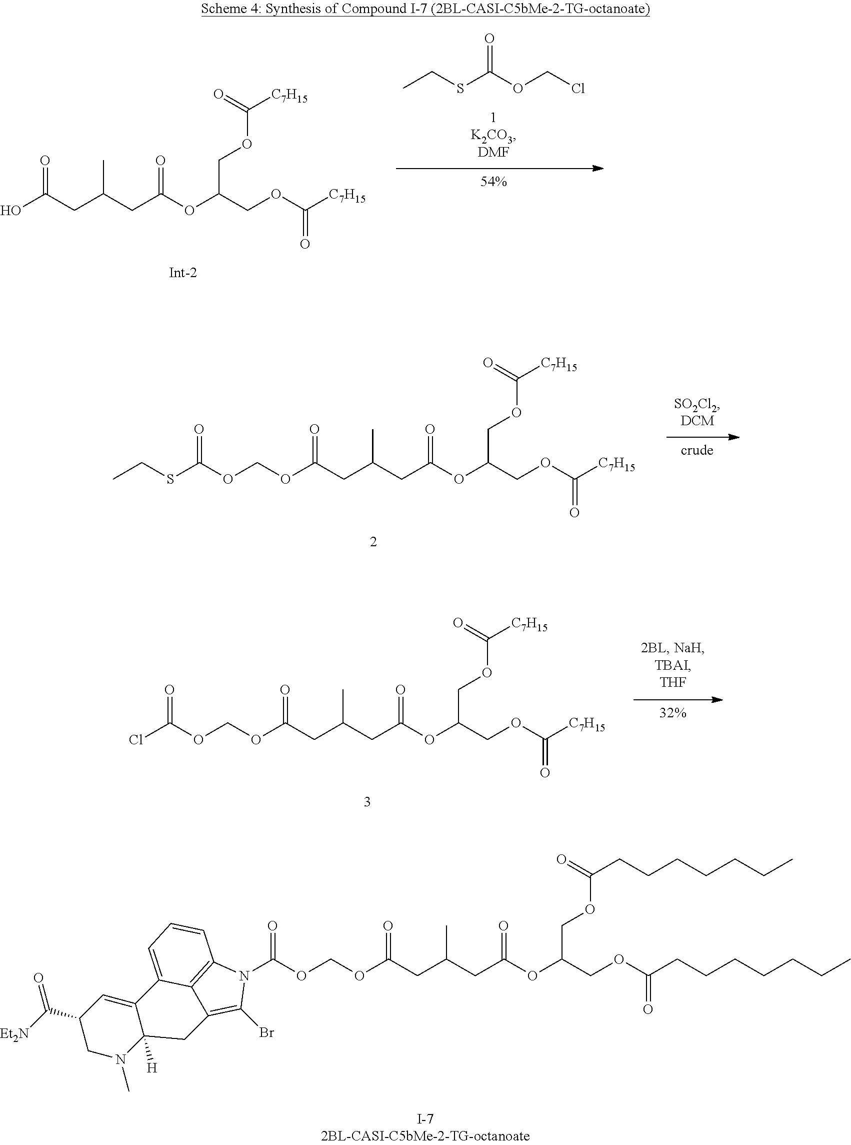

Preparation of O-(chloromethyl)S-ethyl carbonothioate reagent 1. To a solution of ethanethiol (739 mg, 11.9 mmol, 0.880 mL) and Et₃N (1.32 g, 13.1 mmol, 1.82 mL) in diethyl ether (30 mL) at 0° C. was added chloromethyl chloroformate (1.53 g, 11.9 mmol, 1.06 mL) dropwise. A precipitate formed and additional diethyl ether (30 mL) was added and the mixture was stirred vigorously. The mixture was allowed to warm to RT and was stirred for 66 hours. The mixture was filtered through celite and washed with diethyl ether (100 mL). The filtrate was concentrated to afford the reagent (1.72 g, crude) as colorless liquid. The reagent was used in the next step without any further purification.

¹H NMR (400 MHZ, CDCl₃): δ 5.77 (s, 2H), 2.97-2.89 (m, 2H), 1.34 (t, J=7.5 Hz, 3H).

A solution of Int-2 (669 mg, 1.42 mmol) and potassium carbonate (978 mg, 7.08 mmol) in DMF (10 mL) was stirred at RT for 30 minutes. A solution of O-(chloromethyl)S-ethyl carbonothioate 1 (300 mg, 1.94 mmol) in DMF (3 mL) was added to the stirring mixture and was heated to 65° C. for 3 hours. The mixture was diluted with water (30 mL) and extracted with ethyl acetate (3×30 mL). The organic fractions were combined, washed with water (90 mL), brine (90 mL), dried (MgSO₄), filtered and concentrated. The material was purified by normal phase chromatography (Biotage Isolera, 25 g SiliaSep cartridge; eluent 0-25% ethyl acetate in heptane over 0% 3 CVs, 0-25% 13 CVs, 25% 2 CVs) to afford 2 (458 mg, 54% yield) as colorless oil.

UPLC4-MS (XB BEH300 C4 20 to 95%): Rₜ=5.08 min., 96.8% (UV), 98.5% (ELSD); MS(ESIpos): m/z=609.1 [M+NH₄]⁺.

¹H NMR (400 MHZ, CDCl₃): δ 5.81 (s, 2H), 5.38-5.20 (m, 1H), 4.38-4.25 (m, 2H), 4.13 (dd, J=11.9, 6.1 Hz, 2H), 2.89 (q, J=7.5 Hz, 2H), 2.53-2.38 (m, 3H), 2.38-2.23 (m, 6H), 1.68-1.53 (m, 4H), 1.38-1.22 (m, 19H), 1.07-1.00 (m, 3H), 0.93-0.84 (m, 6H).

To a stirring solution of 2 (237 mg, 401 μmol) in DCM (9 mL) was added sulfuryl chloride (1 M, 4.10 mmol, 4.10 mL) dropwise and the mixture was heated to 40° C. for 3 hours. The mixture was allowed to cool to RT, then concentrated to afford 3 (244 mg, crude) as a yellow oil. The product was used in the next step without any further purification.

¹H NMR (400 MHZ, CDCl₃): δ 5.82 (s, 2H), 5.31-5.25 (m, 1H), 4.31 (ddd, J=11.9, 4.1, 0.9 Hz, 2H), 4.14 (dd, J=11.9, 6.0 Hz, 2H), 2.59-2.26 (m, 9H), 1.69-1.56 (m, 4H), 1.37-1.22 (m, 16H), 1.13-1.03 (m, 3H), 0.93-0.83 (m, 6H).

A suspension of 2BL (83.0 mg, 206 μmol), tetrabutylammonium iodide (TBAI, 40.0 mg, 108 μmol) and NaH (17.0 mg, 425 μmol, 60% w/w dispersion in mineral oil) in tetrahydrofuran (5 mL) at 0° C. was stirred for 10 minutes. 3 (234 mg, 414 μmol) in tetrahydrofuran (2 mL) was added and the mixture was warmed to RT and stirred for 18 hours. The mixture was diluted with water (20 mL) and extracted with ethyl acetate (3×20 mL). The combined organic fractions were washed with brine (60 mL), dried (MgSO₄), filtered and concentrated. The material was purified by normal phase chromatography Biotage Isolera, 4 g SiliaSep cartridge; eluent 50-100% ethyl acetate (+0.5% Et₃N) in heptane (+0.5% Et₃N) over 2 CVs 50%, 20 CVs 50-100%, 5 CVs 100%) to afford 2BL-CASI-C5bMe-2-TG-octanoate, Compound I-7 (62.0 mg, 32% yield) as a yellow gum.

UPLC4-MS (XB BEH300 C4 20 to 95%): Rₜ=5.23 min., 93.5% (UV), 98.1% (ELSD); MS(ESIpos): m/z=930.7, 932.7 [M+H]+−79Br/81Br isotope pattern.

¹H NMR (400 MHZ, CDCl₃): δ 7.76 (d, J=8.2 Hz, 1H), 7.37 (d, J=7.6 Hz, 1H), 7.31-7.24 (m, 1H), 6.36 (s, 1H), 6.08 (s, 2H), 5.34-5.18 (m, 1H), 4.32-4.24 (m, 2H), 4.11 (dd, J=11.8, 5.9 Hz, 2H), 3.95-3.77 (m, 1H), 3.52-3.32 (m, 5H), 3.24-3.10 (m, 1H), 3.04 (br dd, J=11.1, 4.7 Hz, 1H), 2.90-2.77 (m, 1H), 2.60 (s, 3H), 2.57-2.33 (m, 5H), 2.29 (s, 5H), 1.65-1.54 (m, 4H), 1.33-1.22 (m, 19H), 1.17 (t, J=7.1 Hz, 3H), 1.03 (d, J=6.4 Hz, 3H), 0.90-0.82 (m, 6H).

¹³C NMR (100 MHZ, CDCl₃): δ 173.4, 171.4, 171.3, 171.0, 149.7, 134.7, 126.8, 126.3, 121.9, 117.8, 115.0, 104.5, 80.4, 69.4, 62.2, 61.9, 55.9, 43.9, 42.3, 40.6, 40.5, 40.3, 40.0, 34.1, 31.8, 29.2, 29.0, 27.5, 27.2, 25.0, 22.7, 19.7, 15.1, 14.2, 13.3.

### Synthesis of Compound I-8 (2BL-CASI-C5bMe-2-TG-oleate)

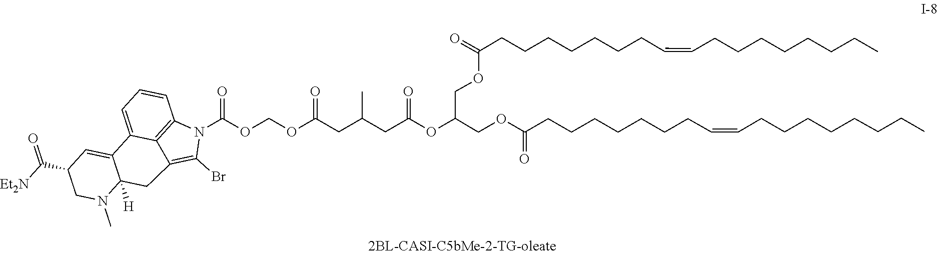

Compound I-8 is prepared similarly to Compound I-7, as described above, from intermediate C5bMe-acid-2-TG-oleate (Int-1), prepared as described in Example 1.

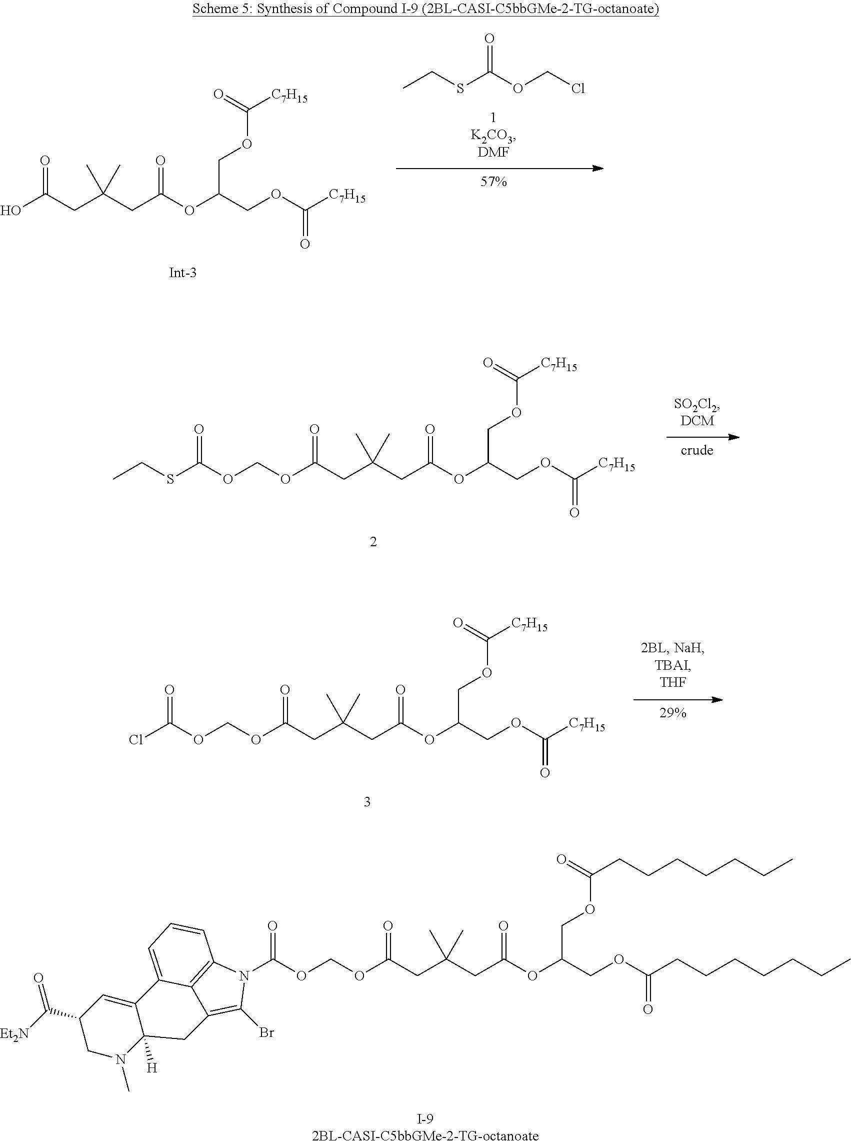

To a solution of Int-3 (500 mg, 1.03 mmol), potassium carbonate (710 mg, 5.14 mmol) in DMF (10 mL) at 0° C., was added a solution of O-(chloromethyl)S-ethyl carbonothioate 1 (243 mg, 1.57 mmol, prepared as previously described) in DMF (2 mL) dropwise and the mixture was stirred at RT for 18 hours. The mixture was diluted with water (30 mL) and was extracted with ethyl acetate (3×30 mL). The organic fractions were combined and washed with water (90 mL), brine (90 mL), dried (MgSO₄), filtered and concentrated. The material was purified by normal phase chromatography (Biotage Isolera, 25 g SiliaSep cartridge; eluent 0-25% ethyl acetate in heptane over 0% for 3 CVs, 0-25% for 13 CVs, 25% for 1 CV) to afford 2 (361 mg, 57% yield) as a colorless oil.

UPLC4-MS (XB BEH300 C4 20 to 95%): Rₜ=5.20 min., 91.6% (UV), 98.3% (ELSD); MS(ESIpos): m/z=622.7 [M+NH₄]+.

¹H NMR (400 MHZ, CDCl₃): δ 5.80 (s, 2H), 5.30-5.23 (m, 1H), 4.36-4.25 (m, 2H), 4.20-4.09 (m, 2H), 2.89 (q, J=7.3 Hz, 2H), 2.55-2.41 (m, 4H), 2.31 (t, J=7.5 Hz, 4H), 1.71-1.57 (m, 4H), 1.36-1.24 (m, 19H), 1.16-1.10 (m, 6H), 0.93-0.83 (m, 6H).

To a stirring solution of 2 (270 mg, 446 μmol) in DCM (10 mL) under argon was added sulfuryl chloride (1 M in DCM, 4.50 mmol, 4.50 mL) and the mixture was heated to 40° C. for 3 hours. The mixture was concentrated to afford 3 (274 mg, crude) as a yellow oil. The material was used in the next step without any further purification.

¹H NMR (400 MHZ, CDCl₃): δ 5.82 (s, 2H), 5.29-5.25 (m, 1H), 4.33-4.25 (m, 2H), 4.17-4.12 (m, 2H), 2.54 (s, 2H), 2.46 (s, 2H), 2.31 (t, J=7.6 Hz, 4H), 1.67-1.56 (m, 4H), 1.35-1.24 (m, 16H), 1.15-1.10 (m, 6H), 0.91-0.85 (m, 6H).

A suspension of 2BL (90.0 mg, 224 μmol), TBAI (43.0 mg, 116 μmol) and NaH (18.0 mg, 450 μmol, 60% w/w dispersion in mineral oil) in tetrahydrofuran (5 mL) at 0° C. was stirred for 10 minutes. 4 (260 mg, 449 μmol) in tetrahydrofuran (2 mL) was added and the mixture was gradually warmed to RT and stirred for 18 hours. The mixture was diluted with water (20 mL) and extracted with ethyl acetate (3×20 mL). The combined organic fractions were washed with brine (60 mL), dried (MgSO₄), filtered and concentrated. The material was purified by normal phase chromatography (Biotage Isolera, 4 g SiliaSep cartridge; eluent 50-100% ethyl acetate (+0.5% Et₃N) in heptane (+0.5% Et₃N) over 2 CVs 50%, 20 CVs 50-100%, 5 CVs 100%) to afford 2BL-CASI-C5bbGMe-2-TG-octanoate, Compound I-9 (60.0 mg, 28% yield) as a yellow gum.

UPLC4-MS (XB BEH300 C4 20 to 95%): Rₜ=5.36 min., 87.5% (UV), 97.5% (ELSD); MS(ESIpos): m/z=944.7, 946.6 [M+H]+; 79Br/81Br isotope pattern.

¹H NMR (400 MHZ, CDCl₃): δ 7.76 (d, J=8.2 Hz, 1H), 7.37 (d, J=7.6 Hz, 1H), 7.30-7.24 (m, 1H), 6.36 (s, 1H), 6.06 (s, 2H), 5.31-5.20 (m, 1H), 4.27 (dd, J=11.9, 4.1 Hz, 2H), 4.11 (m, J=11.9, 6.0 Hz, 2H), 3.91-3.82 (m, 1H), 3.53-3.32 (m, 5H), 3.21-3.11 (m, 1H), 3.04 (dd, J=11.4, 4.9 Hz, 1H), 2.90-2.79 (m, 1H), 2.60 (s, 3H), 2.54 (s, 2H), 2.49-2.39 (m, 3H), 2.34-2.25 (m, 4H), 1.65-1.55 (m, 4H), 1.34-1.22 (m, 19H), 1.20-1.14 (m, 3H), 1.14-1.10 (m, 6H), 0.91-0.84 (m, 6H).

¹³C NMR (100 MHZ, CDCl₃): δ 173.4, 171.4, 170.8, 170.5, 149.7, 134.64, 134.59, 127.7, 126.8, 126.2, 121.9, 120.9, 117.8, 114.9, 104.5, 80.3, 69.1, 62.2, 61.9, 55.9, 45.1, 44.5, 44.0, 42.3, 40.5, 40.0, 34.1, 32.8, 31.8, 29.2, 29.1, 27.74, 27.65, 27.5, 24.98, 24.95, 22.8, 15.1, 14.2, 13.3.

### Alternate Route for Synthesis of Compound I-9 (2BL-CASI-C5bbGMe-2-TG-octanoate)

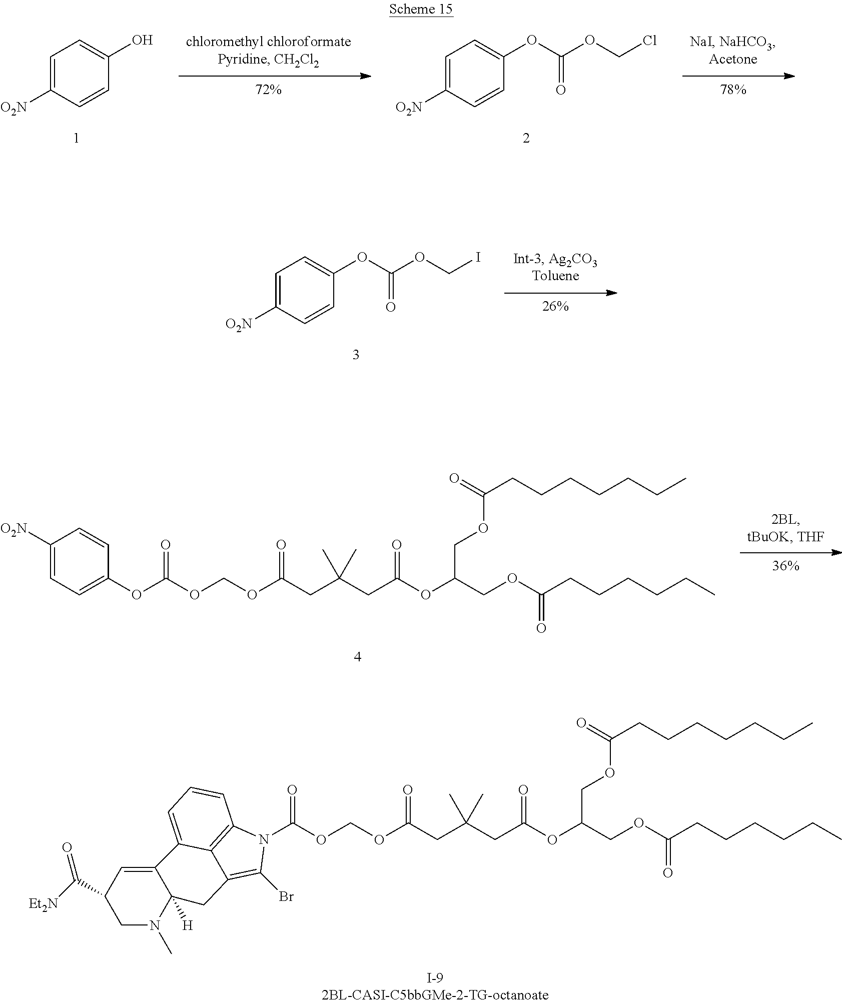

A solution of 4-nitrophenol (3.5 g, 25.2 mmol) and pyridine (2.05 mL, 25.5 mmol) in dichloromethane (25 mL) was added dropwise over 30 minutes to a solution of chloromethyl chloroformate (2.5 mL, 28.1 mmol) in dichloromethane (50 mL) at 0° C. The mixture was stirred at 0° C. for 2 hours then quenched with water (75 mL). The organic layer was washed with 1M aqueous sodium hydroxide solution (30 mL), brine (50 mL), dried over MgSO₄, filtered and concentrated to yield chloromethyl (4-nitrophenyl) carbonate (2) as a yellow solid (4.55 g, 72% yield).

¹H NMR (400 MHZ, CDCl₃): δ [ppm]=8.32-8.29 (m, 2H), 7.45-7.41 (m, 2H), 5.85 (s, 2H).

To a mixture of chloromethyl (4-nitrophenyl) carbonate (2) (2.5 g, 10.8 mmol), sodium iodide (3.24 g, 21.6 mmol), sodium bicarbonate (181 mg, 2.15 mmol), and acetone (25 mL) were heated at 40° C. for 18 hours. The reaction was cooled to ambient temperature, filtered, and washed with acetone (70 mL). The filtrate was concentrated, dissolved in diethyl ether (50 mL), and washed with water (50 mL). The organic extract was washed with 10% aqueous sodium thiosulfate solution (50 mL), brine (50 mL), dried under MgSO₄, filtered and concentrated to provide crude (3) as a yellow oil (2.77 g, 78% yield).

¹H NMR (400 MHZ, CDCl₃): δ [ppm]=8.33-8.29 (m, 2H), 7.45-7.40 (m, 2H), 6.07 (s, 2H).

A solution of Int-3 (1.6 g, 3.29 mmol) in toluene (5 mL) was added to a suspension of iodomethyl (4-nitrophenyl) carbonate (1 g, 3.10 mmol) and silver carbonate (1.2 g, 4.35 mmol) in toluene (15 mL) and stirred under an atmosphere of argon without light at 50° C. for 3 hours. The reaction was quenched with water (60 mL) and extracted into ethyl acetate (3×60 mL). The combined organic extracts were washed with brine (150 mL), dried under MgSO₄, filtered, and concentrated. Material was purified by normal phase chromatography (BioTage Isolera, 25 g Siliasep cartridge) using an eluent of 0-25% ethyl acetate in heptane to yield desired product (720 mg, 25% yield) as a white wax.

¹H NMR (400 MHZ, CDCl₃): δ [ppm]=8.31-8.27 (m, 2H), 7.43-7.39 (m, 2H), 5.87 (s, 2H), 5.31-5.22 (m, 1H), 4.29 (dd, J=4.2, 11.9 Hz, 2H), 4.14 (dd, J=6.1, 11.9 Hz, 2H), 2.55 (s, 2H), 2.47 (s, 2H), 2.30 (t, J=7.5 Hz, 4H), 1.66-1.54 (m, 4H), 1.40-1.21 (m, 16H), 1.15 (s, 6H), 0.87 (t, J=6.8 Hz, 6H).

Potassium tert-butoxide (150 mg, 1.34 mmol) was added to a solution of 2BL (385 mg, 957 μmol) in tetrahydrofuran (THF, 10 mL) at 0° C. under an atmosphere of argon and stirred for 30 minutes. A solution of 1-(1,3-bis(octanoyloxy) propan-2yl) 5-(((4-nitrophenoxy) carbonyl)oxy)methyl) 3,3-dimethylpentanedioate (695 mg, 1.02 mmol) in THF (5 mL) was added to the mixture, gradually warmed to RT, and stirred for 18 hours. The reaction was quenched with a saturated aqueous ammonium chloride solution (50 mL) and extracted into ethyl acetate (3×50 mL). The combined organic extracts were washed with brine (150 mL), dried under MgSO₄, filtered, and concentrated. Material was purified by normal phase purification (Biotage Isolera, 40 g SilliCycle cartridge) using an eluent of 50-100% ethyl acetate+0.5% Et₃N in heptane to yield 2BL-CASI-C5bbGMe-2-TG-octanoate (324 mg, 36% yield) as a yellow gum.

¹H NMR (400 MHZ, CDCl₃): δ [ppm]=7.77 (d, J=8.2 Hz, 1H), 7.37 (d, J=7.6 Hz, 1H), 7.29-7.25 (m, 1H), 6.36 (s, 1H), 6.07 (s, 2H), 5.27-5.22 (m, 1H), 4.27 (dd, J=4.2, 12.0 Hz, 2H), 4.12 (dd, J=6.1, 12.0 Hz, 2H), 3.87-3.85 (m, 1H), 3.49-3.35 (m, 5H), 3.17-3.13 (m, 1H), 3.06-3.03 (m, 1H), 2.87-2.83 (m, 1H), 2.60 (br s, 3H), 2.54 (s, 2H), 2.46-2.41 (m, 3H), 2.29 (t, J=7.6 Hz, 4H), 1.61-1.57 (m, 4H), 1.27-1.24 (m, 19H), 1.17 (t, J=7.1 Hz, 3H), 1.12 (s, 6H), 0.87 (t, J=6.6 Hz, 6H).

¹³C NMR (100 MHZ, CDCl₃): δ [ppm]=173.4, 170.8, 170.5, 149.7, 134.7, 126.9, 126.2, 121.9, 117.8, 115.0, 80.3, 69.1, 62.2, 61.9, 55.9, 45.1, 44.6, 43.9, 42.3, 40.5, 34.1, 32.8, 32.0, 31.8, 29.2, 29.0, 27.7, 25.0, 22.8, 22.7, 15.1, 14.3, 14.2, 13.3

### Synthesis of Compound I-9′ (2BL-CASI-C5bcPr-2-TG-octanoate)

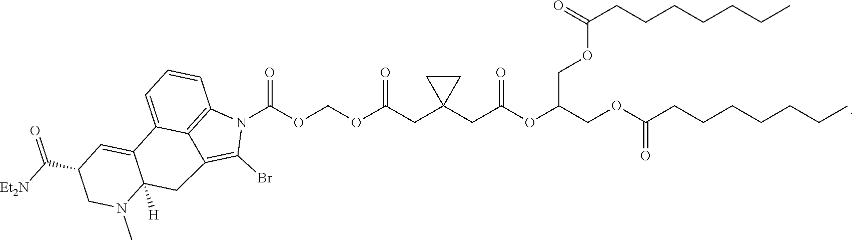

Compound I-9′ was prepared similarly to Compound I-9, above, using intermediate C5bcPr-acid-2-TG-octanoate (Int-5) instead of Int-3.

### Synthesis of Compound I-10 (2BL-CASI-C10b′bMe-2-TG-oleate)

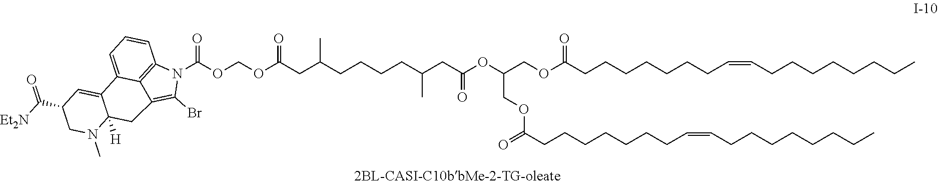

Compound I-10 is prepared similarly to Compound I-9, above, using intermediate C10b′bMe-acid-2-TG-oleate (Int-19) instead of Int-3.

### Synthesis of Compound I-11 (2BL-CMSI-C5bMe-2-TG-oleate)

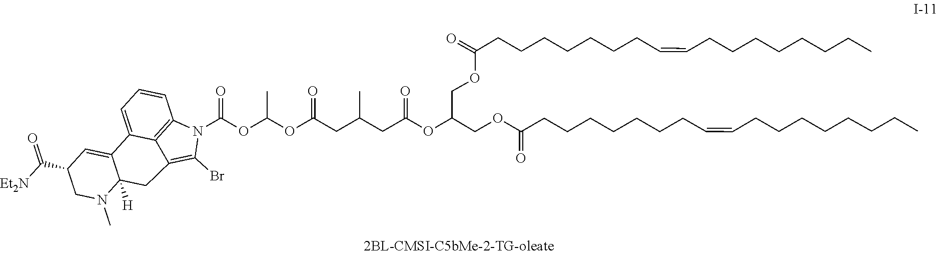

Compound I-11 is prepared similarly to Compound I-8, above, using 1-chloroethyl chloroformate instead of chloromethyl chloroformate in the initial step.

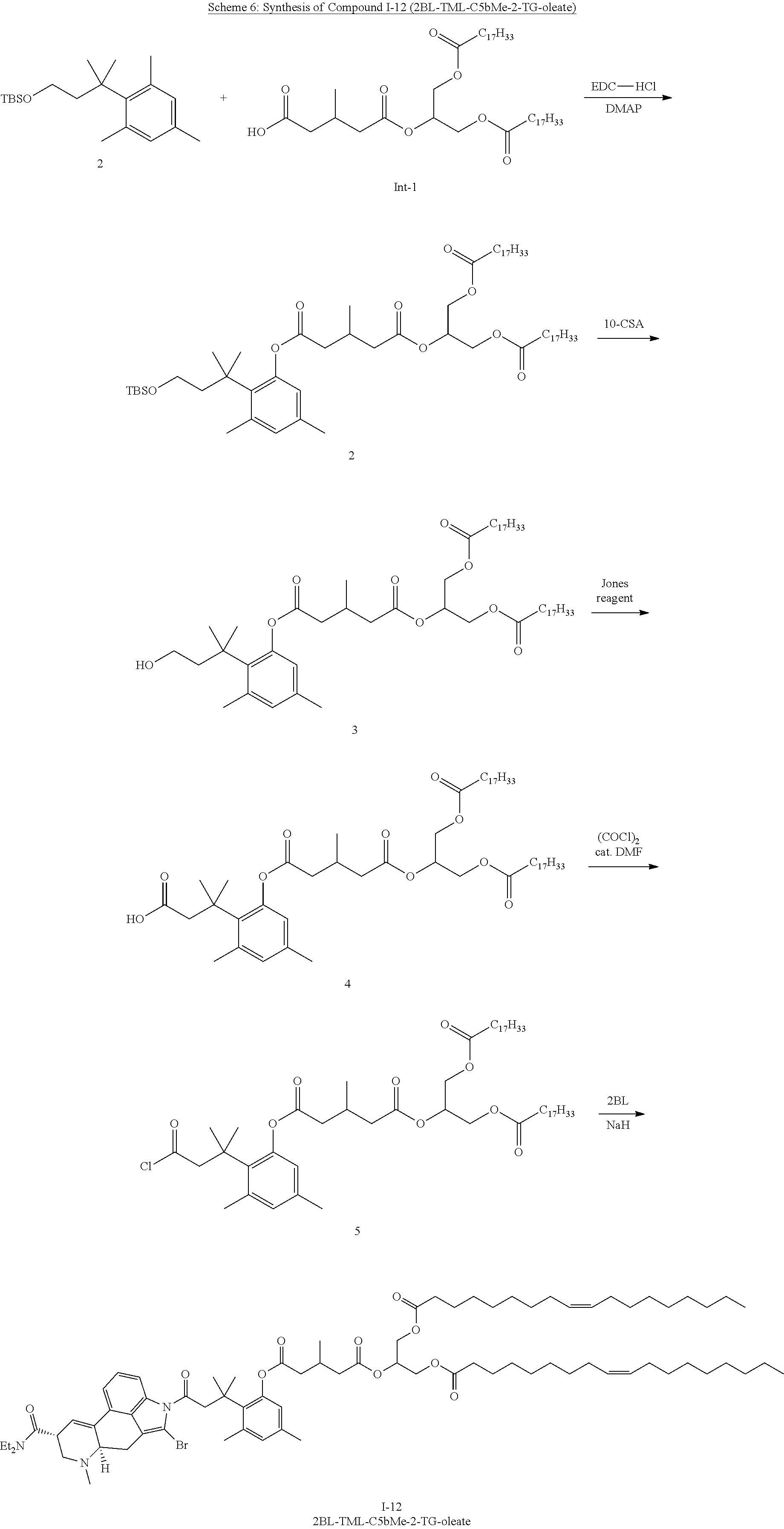

To a solution of 1 (310 mg, 1.2 eq, 961 μmol) and Int-1 (0.6 g, 1 eq, 801 μmol) in DCM (17.8 mL, 0.045 M), DMAP (97.9 mg, 801 μmol) and EDC-HCl (384 mg, 2.5 eq, 2 mmol) were added under nitrogen, and the reaction stirred at RT over two nights. The reacted mixture was diluted with DCM (90 mL) and evaporated onto silica. The crude material was purified by column chromatography to give 2 (616 mg, 585 μmol, 73%) as a clear oil.

¹H NMR (400 MHZ, CDCl₃): δ 6.80 (dt, J=2.1, 0.7 Hz, 1H), 6.52 (dt, J=2.1, 0.7 Hz, 1H), 5.41-5.25 (m, 5H), 4.31 (ddd, J=11.9, 4.3, 2.5 Hz, 2H), 4.14 (dd, J=11.9, 6.0 Hz, 2H), 3.64 (s, 2H), 3.47 (dd, J=8.2, 6.9 Hz, 2H), 2.68-2.42 (m, 7H), 2.39-2.26 (m, 5H), 2.23 (d, J=0.7 Hz, 3H), 2.08-1.96 (m, 10H), 1.59 (q, J=6.5 Hz, 6H), 1.41-1.23 (m, 47H), 1.13 (d, J=6.4 Hz, 2H), 0.86 (d, J=15.4 Hz, 16H), -0.03 (s, 6H).

LRMS (ESI)⁺ m/z calculated for C₆₄H₁₁₂O₉Si [M+NH₄]⁺: 1070.8; found: 1071.

In a 20 mL crimp-top vial with stir bar under nitrogen, 2 (523 mg, 496 μmol) was dissolved in methanol (4.96 mL, 0.1 M) and DCM (4.96 mL, 0.1 M), then cooled in a water/ice bath. Camphorsulfonic acid (10-CSA, 17.2 mg, 0.15 eq, 74.5 μmol) was added all at once, and the reaction was stirred for around 30 minutes before removing the ice bath and continuing to stir. The reaction was diluted with DCM (10 mL) and washed with saturated aqueous sodium bicarbonate solution (2×5 mL). The organic portion was dried with sodium sulfate, filtered, and evaporated to give the crude material. The material was dry loaded onto silica and purified by column chromatography (0-30% ethyl acetate/heptanes) to give 3 (435 mg, 463 μmol, 93%) as a clear oil.

¹H NMR (400 MHZ, CDCl₃): δ 6.82 (d, J=2.1 Hz, 1H), 6.53 (d, J=2.1 Hz, 1H), 5.40-5.25 (m, 5H), 4.32 (ddd, J=11.9, 4.3, 1.5 Hz, 2H), 4.15 (dd, J=11.9, 6.0 Hz, 2H), 3.53 (t, J=7.3 Hz, 2H), 2.72-2.44 (m, 7H), 2.42-2.26 (m, 5H), 2.23 (s, 3H), 2.10-1.94 (m, 9H), 1.66-1.37 (m, 12H), 1.36-1.23 (m, 44H), 1.14 (d, J=6.4 Hz, 3H), 0.92-0.84 (m, 7H).

LRMS (ESI)⁺ m/z calculated for C₅₈H₉₈O₉ [M+NH₄]⁺: 956.8; found: 957.1.

In a 100 mL RBF with stir bar, 3 (403 mg, 429 μmol) was dissolved in acetone (6.36 mL, 0.067 M) and cooled to 0° C. in a water/ice bath. Jones reagent (2 M CrO₃ in H₂SO₄) (0.29 mL, 1.34 eq, 575 μmol) was added dropwise and the reaction stirred at 0° C. for four hours. The reaction was quenched with water (5.5 mL) and extracted with ethyl acetate (3×5.5 mL). The combined organic extracts were dried with sodium sulfate, filtered, and evaporated to give the crude material which was dry loaded onto silica. Purification by column chromatography (5-40% ethyl acetate/heptanes) gave 4 (310 mg, 325 μmol, 76%) as a clear oil.

¹H NMR (400 MHZ, CDCl₃): δ 6.82 (d, J=2.1 Hz, 1H), 6.56 (d, J=2.0 Hz, 1H), 5.41-5.23 (m, 5H), 4.31 (dd, J=11.9, 4.4 Hz, 2H), 4.16 (dd, J=11.9, 5.9 Hz, 2H), 2.82 (d, J=1.3 Hz, 2H), 2.67 (dd, J=15.5, 5.9 Hz, 1H), 2.64-2.47 (m, 6H), 2.37 (dd, J=15.2, 7.1 Hz, 1H), 2.31 (td, J=7.6, 1.7 Hz, 4H), 2.23 (s, 3H), 2.04-1.97 (m, 8H), 1.63-1.55 (m, 11H), 1.36-1.22 (m, 47H), 1.13 (d, J=6.6 Hz, 3H), 0.94-0.84 (m, 6H).

LRMS (ESI)+m/z calculated for C₅₈H₉₆O₁₀ [M+NH₄]⁺: 970.7; found: 971.0.

Oxalyl chloride (1.5 eq) and DMF (1-2 drops, catalytic) are added to 4 (1 eq) in DCM (0.1 M) at 0° C., and the resulting mixture is stirred at 0° C. for 1-2 hours. The reaction is concentrated under reduced pressure, and the resulting residue re-evaporated 3-4 times from toluene to give 5, which is used in the next step without further purification.

A solution of 2BL (1 eq) in THF (0.1 M) is added to a suspension of NaH (60% w/w dispersion in mineral oil, 1 eq) in THF (0.1 M) at 0° C. and the reaction is stirred at 0° C. for around 30 minutes. A solution of 5 (1.2 eq) in THF (0.1 M) is slowly added, and the resulting mixture is stirred at 0° C. for around 30 minutes, then allowed to warm to RT and is stirred at RT for 4-24 hours. The reaction is quenched by the slow addition of saturated aqueous NH₄Cl, diluted with water and extracted with ethyl acetate. The combined organic extracts are washed with water and brine, dried with MgSO₄, and concentrated under reduced pressure to give the crude reaction mixture. Purification by silica gel chromatography with a suitable solvent mixture affords 2BL-TML-C5bMe-2-TG-oleate, Compound I-12.

### Synthesis of Compound 1-13 (2BL-TML-C8bMe-2-TG-oleate)

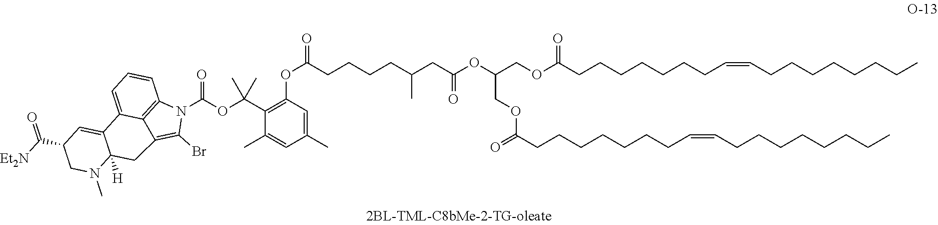

Compound I-13 is prepared similarly to Compound I-12, as described above, from intermediate C8bMe-acid-2-TG-oleate (Int-16), prepared as described in Example 1.

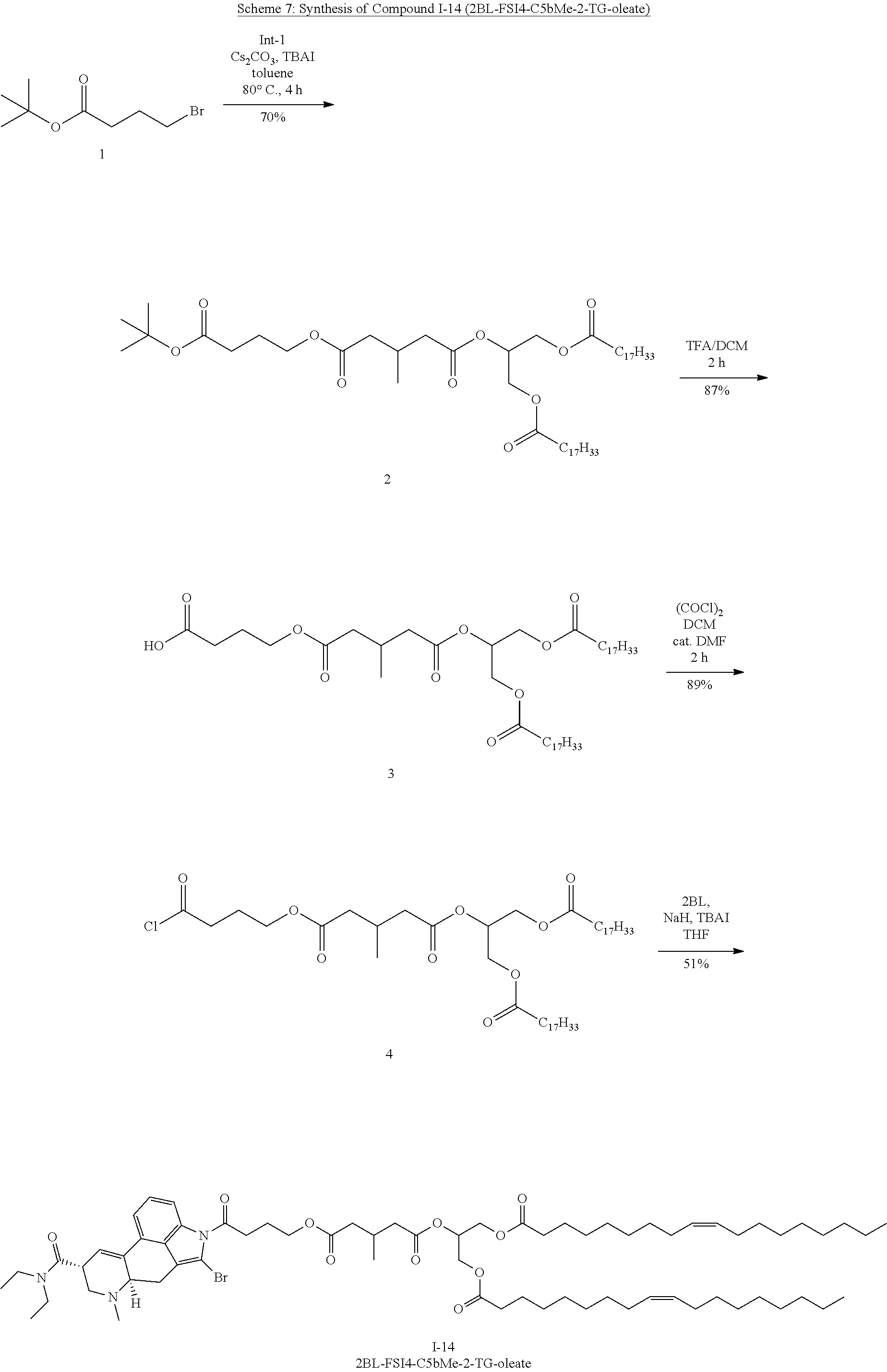

To a stirring solution of Int-1 (2.00 g, 2.67 mmol) and tert-butyl 4-bromobutanoate 1 (596 mg, 2.67 mmol, 474 μL) in toluene (10 mL) was added TBAI (494 mg, 1.34 mmol) and cesium carbonate (2.61 g, 8.01 mmol) at RT. The reaction was heated to 80° C. and stirred 4 hours. The reaction mixture was diluted with ethyl acetate and the organics were washed with brine, dried over sodium sulfate, filtered and concentrated to provide the crude product. The material was purified by normal phase purification (Biotage Isolera, 80 g SiliaSep cartridge; eluent 0-30% ethyl acetate in heptane) to afford 2 (1.66 g, 69.8% yield) as a colorless oil.

¹H NMR (400 MHZ, CDCl₃): δ 5.39-5.24 (m, 5H), 4.30 (ddd, J=3.2, 5.7, 9.3 Hz, 2H), 4.16-4.08 (m, 4H), 2.51-2.35 (m, 3H), 2.33-2.19 (m, 8H), 2.06-1.96 (m, 8H), 1.95-1.87 (m, 2H), 1.65-1.57 (m, 4H), 1.44 (s, 9H), 1.37-1.20 (m, 40H), 1.02 (d, J=6.7 Hz, 3H), 0.88 (t, J=6.6 Hz, 6H).

¹³C NMR (101 MHZ, CDCl₃): δ 173.4, 172.3, 172.2, 171.5, 130.2, 129.9, 80.6, 69.2, 63.6, 62.2, 40.82, 40.78, 34.1, 32.08, 32.05, 29.91, 29.85, 29.7, 29.5, 29.32, 29.26, 29.2, 28.2, 27.5, 27.4, 27.3, 25.0, 24.3, 22.8, 19.8, 14.3.

To a stirring solution of 2 (500 mg, 561 μmol) in DCM (8 mL), trifluoroacetic acid (TFA, 2.98 g, 26.1 mmol, 2.00 mL) was added at RT. The reaction was stirred at RT for 2 hours. The reaction mixture was diluted with ethyl acetate (50 mL) washed with aqueous sodium bicarbonate (3×50 mL) followed by a washing with water (2×50 mL) followed by brine (3×50 mL). The organic layer was dried over sodium sulfate, filtered and concentrated in vacuo to afford 3 (405 mg, 86.5% yield) as a colorless oil.

UPLC4-MS: (XB BEH 300 C4 20-95%): Rₜ=6.30 min., 60.7% (UV), 93.8% (ELSD). MS (ESIpos): m/z=853.0 [M+NH₄]⁺.

¹H NMR (400 MHZ, CDCl₃): δ 5.55-5.00 (m, 5H), 4.29 (dd, J=4.2, 11.9 Hz, 2H), 4.18-4.06 (m, 4H), 2.51-2.35 (m, 5H), 2.33-2.19 (m, 6H), 2.16-1.77 (m, 10H), 1.64-1.55 (m, 4H), 1.27 (m, 40H), 1.01 (d, J=6.4 Hz, 3H), 0.86 (br t, J=6.7 Hz, 6H); exchangeable CO₂H proton not observed.

¹³C NMR (101 MHZ, CDCl₃): δ 173.46, 173.44, 172.2, 171.6, 130.2, 129.9, 69.3, 63.4, 62.2, 40.8, 40.7, 34.1, 32.0, 30.6, 29.9, 29.83, 29.79, 29.7, 29.5, 29.4, 29.3, 29.25, 29.22, 27.5, 27.4, 27.3, 25.0, 24.0, 22.8, 19.8, 14.2.

To a stirring solution of 3 (400 mg, 479 μmol) in DCM (8 mL) was added oxalyl chloride (76.0 mg, 599 μmol, 50.7 μL) and DMF (3 drops, catalytic) at RT. The reaction was stirred at RT for 2 hours. The reaction mixture was concentrated in vacuo to afford 4 (406 mg, 88.4% yield) as a yellow oil.

¹H NMR (400 MHZ, CDCl₃): δ 5.64-5.01 (m, 5H), 4.37-4.23 (m, 2H), 4.21-4.05 (m, 4H), 3.00-2.95 (m, 3H), 2.52-2.35 (m, 3H), 2.35-2.18 (m, 6H), 2.08-1.95 (m, 9H), 1.60 (br t, J=6.8 Hz, 4H), 1.37-1.20 (m, 40H), 1.02 (d, J=6.4 Hz, 3H), 0.87 (t, J=6.7 Hz, 6H).

To a stirring solution of 2BL (94.0 mg, 234 μmol) in tetrahydrofuran (4 mL) was added NaH (12.0 mg, 300 μmol, 60% purity) and TBAI (44.0 mg, 119 μmol) at 0° C. The solution was stirred for 10 minutes at 0° C. 4 (400 mg, 469 μmol) in tetrahydrofuran (4 mL) was then added at 0° C. The solution was allowed to warm to RT and stirred for 3 hours. The reaction mixture was diluted with ethyl acetate (50 mL) washed with water (50 mL) followed by a wash with brine (50 mL). The organic layer was dried over sodium sulfate, filtered and concentrated in vacuo to afford a crude yellow oil. The material was purified by normal phase purification (Biotage Isolera, 25 g SiliCycle cartridge; eluent 0-80% ethyl acetate+0.5% Et₃N in heptane+0.5% Et₃N over 30 CVs) to afford 2BL-FSI4-C5bMe-2-TG-oleate, Compound I-14 (147 mg, 51.3% yield) as a yellow oil.

UPLC4-MS: (XB BEH 300 C4 20-95%): Rₜ=7.10 min, 78.4% (UV), 92.3% (ELSD). MS (ESIpos): m/z=1219.0, 1220.9 [M+H]+ 79Br/81Br isotope pair.

¹H NMR (400 MHZ, CDCl₃): δ 7.98 (d, J=8.2 Hz, 1H), 7.38 (d, J=7.6 Hz, 1H), 7.32-7.23 (m, 1H), 6.37 (s, 1H), 5.53-5.01 (m, 5H), 4.34-4.18 (m, 4H), 4.13 (br dd, J=5.9, 11.9 Hz, 2H), 3.88 (br s, 1H), 3.51-3.32 (m, 5H), 3.31-3.14 (m, 3H), 3.06 (br dd, J=4.2, 10.9 Hz, 1H), 2.90-2.74 (m, 1H), 2.61 (s, 3H), 2.52-2.35 (m, 4H), 2.33-2.15 (m, 8H), 2.14-1.79 (m, 8H), 1.64-1.55 (m, 4H), 1.27 (m, 43H), 1.17 (t, J=7.1 Hz, 3H), 1.02 (d, J=6.4 Hz, 3H), 0.87 (t, J=6.6 Hz, 6H).

¹³C NMR (101 MHZ, CDCl₃): δ 173.3, 172.2, 171.8, 171.4, 171.3, 135.6, 134.5, 130.1, 129.8, 128.2, 127.4, 126.52, 126.47, 121.8, 121.0, 117.8, 115.5, 103.1, 69.3, 63.5, 62.2, 62.0, 55.9, 43.9, 42.2, 40.77, 40.74, 39.9, 35.7, 34.1, 32.0, 29.9, 29.82, 29.77, 29.7, 29.6, 29.4, 29.3, 29.23, 29.21, 27.7, 27.5, 27.4, 27.34, 27.29, 24.9, 24.2, 22.8, 19.8, 15.1, 14.2, 13.3.

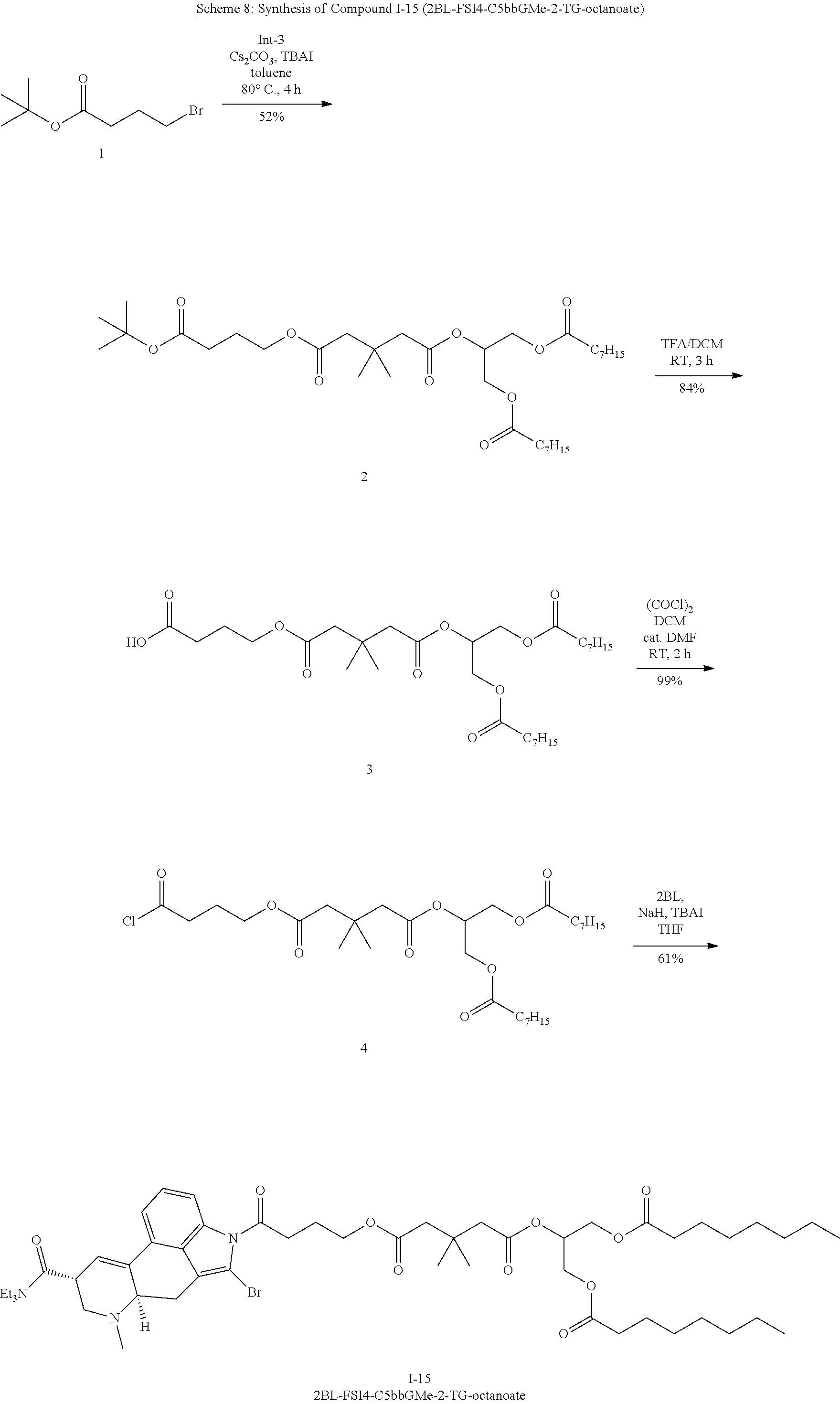

To a stirring solution of Int-3 (750 mg, 1.54 mmol), cesium carbonate (1.51 g, 4.62 mmol) and TBAI (285 mg, 772 μmol) in toluene (15 mL) was added tert-butyl 4-bromobutanoate 1 (344 mg, 1.54 mmol, 273 μL) at RT. The reaction mixture was heated to 80° C. and stirred for 4 hours. The reaction mixture was allowed to cool, and then diluted with ethyl acetate (100 mL), washed with water (100 mL) and brine (2×100 mL). The organic layer was dried over sodium sulfate, filtered and concentrated to give a crude yellow oil. The material was purified by normal phase purification (Biotage Isolera, 80 g SiliCycle cartridge; eluent 0-30% ethyl acetate in heptane over 24 CVs) to afford 2 (505 mg, 52% yield) as a colorless oil.

UPLC4-MS: (XB BEH 300 C4 20-95%): Rt=5.35 min, 97.2% (UV), 99.5% (ELSD). MS (ESIpos): m/z=647.2 [M+NH₄]+.

¹H NMR (400 MHZ, CDCl₃): δ 5.35-5.15 (m, 1H), 4.33-4.22 (m, 2H), 4.13 (dd, J=6.1, 11.9 Hz, 2H), 4.07 (t, J=6.5 Hz, 2H), 2.44 (s, 2H), 2.40 (s, 2H), 2.34-2.10 (m, 6H), 1.91 (quin, J=6.9 Hz, 2H), 1.64-1.55 (m, 4H), 1.44 (s, 9H), 1.27 (m, 16H), 1.11 (s, 6H), 0.87 (t, J=6.4 Hz, 6H).

¹³C NMR (101 MHZ, CDCl₃): δ 173.38, 172.2, 171.71, 171.69, 171.0, 80.59, 69.0, 63.3, 62.3, 45.3, 34.1, 32.7, 32.1, 31.8, 29.2, 29.0, 28.2, 27.6, 24.9, 24.3, 22.7, 14.18.

To a solution of 2 (500 mg, 795 μmol) in DCM (8 mL), TFA (3.00 g, 26.3 mmol, 2.00 mL) was added at RT. The reaction was stirred at RT for 3 hours. The reaction mixture was diluted with ethyl acetate (50 mL) washed with aqueous sodium bicarbonate (3×50 mL) followed by water (2×50 mL) and brine (3×50 mL). The organic layer was dried over sodium sulfate, filtered and concentrated to afford 3 (384 mg, 84% yield) as a colorless oil.

UPLC4-MS: (XB BEH 300 C4 20-95%): Rt=3.61 min, 96.3% (UV), 97.5% (ELSD). MS (ESIpos): m/z=590.7 [M+NH₄]+.

¹H NMR (400 MHZ, CDCl₃): δ 5.34-5.20 (m, 1H), 4.29 (dd, J=4.3, 11.9 Hz, 2H), 4.21-4.04 (m, 4H), 2.48-2.42 (m, 4H), 2.40 (s, 2H), 2.30 (t, J=7.6 Hz, 4H), 1.97 (quin, J=6.8 Hz, 2H), 1.60 (m, 4H), 1.33-1.22 (m, 16H), 1.11 (s, 6H), 0.87 (t, J=6.5 Hz, 6H); exchangeable CO₂H proton not observed.

¹³C NMR (101 MHZ, CDCl₃): δ 173.5, 171.7, 171.0, 69.0, 63.1, 62.3, 45.2, 45.1, 34.2, 32.8, 31.8, 30.6, 29.2, 29.0, 27.7, 25.0, 23.9, 22.7, 14.2.

To a solution of 3 (370 mg, 646 μmol) in DCM (7 mL) was added oxalyl chloride (103 mg, 814 μmol, 71.0 μL) and DMF (3 drops, catalytic) at RT. The reaction was stirred at RT for 2 hours. The reaction mixture was concentrated to afford 4 (379 mg, 99% yield) as a yellow oil.

¹H NMR (400 MHZ, CDCl₃): δ 5.31-5.20 (m, 1H), 4.28 (dd, J=4.3, 11.9 Hz, 2H), 4.19-4.00 (m, 4H), 2.99 (t, J=7.2 Hz, 2H), 2.44 (s, 2H), 2.41 (s, 2H), 2.30 (t, J=7.5 Hz, 4H), 2.03 (quin, J=6.7 Hz, 2H), 1.64-1.55 (m, 4H), 1.33-1.22 (m, 16H), 1.11 (s, 6H), 0.87 (t, J=6.7 Hz, 6H).

¹³C NMR (101 MHZ, CDCl₃): δ 173.39, 173.36, 171.6, 170.9, 69.1, 62.23, 62.18, 45.1, 45.0, 43.9, 34.1, 32.7, 31.8, 29.2, 29.0, 27.7, 25.0, 24.4, 22.7, 14.9.

To a stirring solution of 2BL (128 mg, 318 μmol) in tetrahydrofuran (4 mL) was added sodium hydride (16.0 mg, 400 μmol, 60% w/w dispersion in mineral oil) and TBAI (59.0 mg, 160 μmol) at 0° C. The solution was stirred for 10 minutes at 0° C. 4 (375 mg, 634 μmol) in tetrahydrofuran (4 mL) was then added at 0° C. The solution was allowed to warm to RT and stirred for 3 hours. The reaction mixture was diluted with ethyl acetate (50 mL), washed with water (50 mL) and brine (50 mL). The organic layer was dried over sodium sulfate, filtered and concentrated to give a crude yellow oil. The material was purified by normal phase purification (Biotage Isolera, 25 g SiliCycle cartridge; eluent 0-80% ethyl acetate (+0.5% Et₃N) in heptane (+0.5% Et₃N) over 24 CVs) to afford 2BL-FSI4-C5bbGMe-2-TG-octanoate, Compound I-15 (185 mg, 61% yield) as a yellow oil.

UPLC4-MS: (XB BEH 300 C4 20-95%): Rt=5.43 min, 96.2% (UV), 100.0% (ELSD). MS (ESIpos): m/z=956.8, 958.7 [M+H]+; 79Br/81Br isotope pair.

¹H NMR (400 MHZ, CDCl₃): δ 7.96 (d, J=8.1 Hz, 1H), 7.36 (d, J=7.6 Hz, 1H), 7.30-7.22 (m, 1H), 6.35 (s, 1H), 5.28-5.20 (m, 1H), 4.26 (dd, J=4.2, 11.9 Hz, 2H), 4.20 (t, J=6.3 Hz, 2H), 4.12 (dd, J=6.1, 11.9 Hz, 2H), 3.87 (br d, J=4.5 Hz, 1H), 3.50-3.30 (m, 5H), 3.29-3.13 (m, 3H), 3.05 (br dd, J=4.7, 11.3 Hz, 1H), 2.91-2.79 (m, 1H), 2.60 (s, 3H), 2.50-2.42 (m, 3H), 2.39 (s, 2H), 2.28 (t, J=7.5 Hz, 4H), 2.17 (quin, J=6.7 Hz, 2H), 1.62-1.54 (m, 4H), 1.30-1.21 (m, 19H), 1.16 (t, J=7.1 Hz, 3H), 1.09 (s, 6H), 0.85 (t, J=6.7 Hz, 6H).

¹³C NMR (101 MHZ, CDCl₃): δ 173.4, 171.9, 171.7, 171.3, 170.9, 135.5, 134.5, 127.3, 126.5, 126.4, 121.7, 121.0, 117.8, 115.5, 103.1, 69.0, 63.3, 62.2, 61.9, 55.8, 45.2, 43.9, 42.2, 40.5, 39.9, 35.8, 34.1, 32.7, 31.7, 29.2, 29.0, 27.6, 24.9, 24.2, 22.7, 15.1, 14.2, 13.3.

### Synthesis of Compound I-15′ (2BL-FSI4-C5bcPr-2-TG-octanoate)

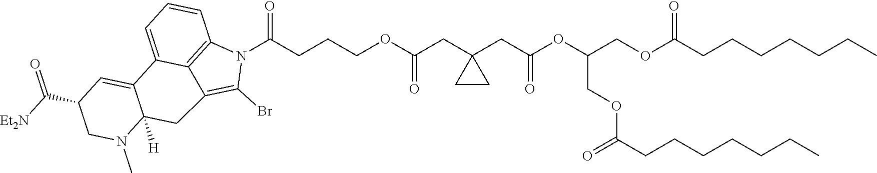

Compound I-15′ was prepared similarly to Compound I-15, above, using intermediate C5bcPr-acid-2-TG-octanoate (Int-5) instead of Int-3.

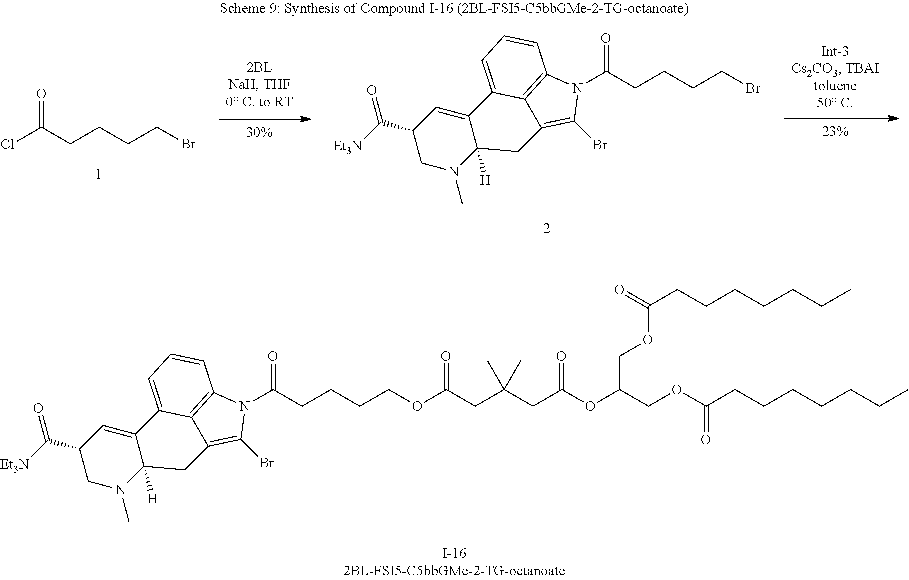

To a stirring solution of 2BL (600 mg, 1.49 mmol) in DMF (10 mL) at 0° C. under argon, was added NaH (89.5 mg, 2.24 mmol, 60% w/w dispersion in mineral oil). The mixture was stirred for 30 minutes while maintaining the temperature at 0° C. 5-bromovaleryl chloride 1 (446 mg, 2.24 mmol, 299 μL) was then added, and the mixture was allowed to warm to RT and stir for 18 hours. The reaction was quenched with saturated aqueous NH₄Cl (50 mL), and the mixture was extracted with diethyl ether (3×50 mL). The combined organic layers were washed with brine (100 mL), dried over MgSO₄, filtered, and concentrated to give a crude oil. The material was purified by normal phase chromatography (Biotage Isolera, 40 g SiliaSep cartridge; eluent 0-10% methanol in DCM over 30 CVs) to afford 2 (505 mg, 30% yield) as a yellow oil.

UPLC6 (BEH C18 Long Neutral 2 to 95): Rt=2.73 min, 51.3% (UV). MS (ESIpos): m/z=566.0, 568.0 [M+H]+.

¹H NMR (400 MHZ, CDCl₃) δ 7.97 (d, J=8.2 Hz, 1H), 7.38 (d, J=7.6 Hz, 1H), 7.31-7.23 (m, 1H), 6.40-6.35 (m, 1H), 3.92 (br s, 1H), 3.61 (t, J=6.3 Hz, 1H), 3.56-3.31 (m, 4H), 3.21 (br t, J=6.2 Hz, 2H), 3.12 (br d, J=4.4 Hz, 1H), 2.97-2.85 (m, 1H), 2.63 (br s, 3H), 2.60-2.46 (m, 2H), 2.42-2.31 (m, 2H), 2.04-1.86 (m, 2H), 1.84-1.74 (m, 2H), 1.26 (t, J=7.1 Hz, 3H), 1.17 (t, J=7.1 Hz, 3H); product contains 5.74% w/w DCM, and an additional impurity, as seen by UPLC.

To a stirring solution of 2 (120 mg, 183 μmol) and Int-3 (115 mg, 237 μmol) in toluene (4 mL) was added cesium carbonate (178 mg, 548 μmol) and TBAI (33.8 mg, 91.4 μmol) at RT. The reaction mixture was heated to 50° C. and stirred for 2 hours. The reaction mixture was diluted with ethyl acetate (25 mL) then washed with water (25 mL), brine (25 mL), dried over sodium sulfate, filtered, and concentrated to give a crude yellow oil. The material was purified by normal phase chromatography (Biotage Isolera, 25 g Silicycle cartridge; eluent 0-100% ethyl acetate in heptane over 30 CVs) to afford 2BL-FSI5-C5bbGMe-2-TG-octanoate, Compound I-16 (40.0 mg, 23% yield) as a yellow oil.

UPLC4 (XB BEH 300 C4 20 to 95): Rt=5.54 min, 92.8% (UV), 99.9% (ELSD). MS (ESIpos): m/z=970.8, 972.7 [M+H]+ 79Br/81Br isotope pair.

¹H NMR (400 MHZ, CDCl₃): δ 7.96 (d, J=8.2 Hz, 1H), 7.36 (d, J=7.6 Hz, 1H), 7.26 (t, J=8.0 Hz, 1H), 6.36 (s, 1H), 5.29-5.23 (m, 1H), 4.27 (dd, J=4.3, 11.9 Hz, 2H), 4.17-4.08 (m, 4H), 3.88 (br s, 1H), 3.50-3.39 (m, 4H), 3.38-3.30 (m, 1H), 3.25-3.12 (m, 3H), 3.05 (br dd, J=4.7, 11.2 Hz, 1H), 2.86 (br t, J=10.9 Hz, 1H), 2.60 (s, 3H), 2.53-2.42 (m, 3H), 2.39 (s, 2H), 2.29 (t, J=7.6 Hz, 4H), 1.97-1.85 (m, 2H), 1.84-1.72 (m, 2H), 1.64-1.53 (m, 4H), 1.33-1.21 (m, 19H), 1.16 (t, J=7.1 Hz, 3H), 1.11 (s, 6H), 0.86 (t, J=6.7 Hz, 6H); 76H expected, 76H observed.

¹³C NMR (101 MHZ, CDCl₃): δ 173.4, 172.3, 171.8, 171.3, 171.0, 135.5, 134.6, 127.4, 126.5, 121.7, 120.9, 117.7, 115.4, 103.1, 69.0, 63.9, 62.2, 62.0, 55.9, 45.3, 45.2, 43.9, 42.2, 40.5, 39.9, 38.6, 34.1, 32.7, 31.8, 29.2, 29.0, 28.2, 27.6, 24.9, 22.7, 21.7, 15.1, 14.2, 13.3.

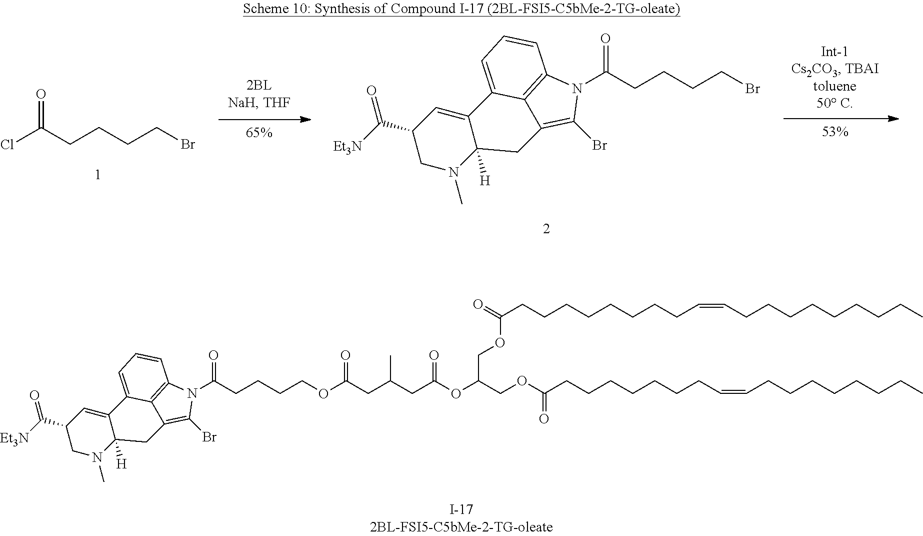

To a stirring solution of 2BL (600 mg, 1.49 mmol) in DMF (10 mL) at 0° C. under argon, was added sodium hydride (89.5 mg, 2.24 mmol, 60% w/w dispersion in mineral oil). The mixture was stirred for 30 mins while maintaining the temperature. 5-bromovaleryl chloride 1 (446 mg, 2.24 mmol, 299 μL) was then added, and the mixture was left to warm to RT and stirred for 4 hours. The material was diluted with diethyl ether (20 mL) and washed with water (2×10 mL), dried over MgSO₄, filtered, and concentrated to give a crude oil. The crude was purified by normal phase chromatography (Biotage Isolera, 40 g SiliaSep cartridge; eluent 0-10% methanol in DCM over 30 CVs) to give 2 (553 mg, 49.2% yield) as a yellow oil.

C_UPLC2-MS: (CSH-C18 Long Neutral 2 to 95%) Rt=1.91 min (75.4%), MS (ESIpos): m/z=[M+H]+ 566, 568.2.

¹H NMR (400 MHZ, CDCl₃): δ 7.40-7.35 (m, 1H), 7.32-7.28 (m, 1H), 7.27-7.25 (m, 1H), 6.38 (s, 1H), 3.61 (t, J=6.2 Hz, 1H), 3.54 (br t, J=6.3 Hz, 1H), 3.50-3.31 (m, 2H), 3.21 (br t, J=6.1 Hz, 3H), 2.92-2.82 (m, 2H), 2.62 (br s, 2H), 2.52-2.32 (m, 2H), 2.11-1.98 (m, 4H), 1.98-1.82 (m, 2H), 1.82-1.73 (m, 2H), 1.26 (br t, J=7.0 Hz, 3H), 1.17 (t, J=7.1 Hz, 3H).

To a solution of Int-1 (585 mg, 782 μmol) and 2 (553 mg, 651 μmol) in toluene (20 mL), cesium carbonate (644 mg, 1.98 mmol) and TBAI (127 mg, 343 μmol) were added at RT. The reaction mixture was heated to 50° C. and was left to stir for 3 hours. The reaction was quenched with a saturated solution of NH₄Cl (100 mL), and the organics were extracted with ethyl acetate (3×20 mL). The combined organic layers were washed with brine (100 mL), dried over MgSO₄ and concentrated under vacuum to afford a crude residue. Material was purified by normal phase purification (Biotage Isolera, 40 g SiliCycle cartridge; eluent 0-100% ethyl acetate in heptane over 40 CVs) to afford 2BL-FSI5-C5bMe-2-TG-oleate, Compound I-17 (429 mg, 53.4% yield) as a yellow oil.

UPLC4-MS (XB BEH300 C4 20 to 95%): Rt=7.15 min., 78.1% (UV), 93.6% (ELSD); MS (ESIpos): m/z=[M+H]+ 1232.96, 1234.91.

¹H NMR (400 MHZ, CDCl₃): δ 7.97 (d, J=8.1 Hz, 1H), 7.37 (d, J=7.6 Hz, 1H), 7.30-7.26 (m, 1H), 6.37 (s, 1H), 5.38-5.24 (m, 5H), 4.32-4.26 (m, 2H), 4.18-4.10 (m, 4H), 3.85 (br s, 1H), 3.51-3.40 (m, 4H), 3.36 (br dd, J=5.7, 15.5 Hz, 1H), 3.26-3.14 (m, 3H), 3.09-3.01 (m, 1H), 2.89-2.82 (m, 1H), 2.60 (s, 3H), 2.49-2.34 (m, 4H), 2.34-2.20 (m, 6H), 2.05-1.95 (m, 8H), 1.95-1.88 (m, 2H), 1.88-1.72 (m, 2H), 1.65-1.54 (m, 4H), 1.28 (br d, J=12.8 Hz, 44H), 1.17 (t, J=7.1 Hz, 3H), 1.02 (d, J=6.5 Hz, 3H), 0.92-0.83 (m, 6H).

¹³C NMR (100 MHz, CDCl₃): δ 173.4, 172.4, 171.5, 171.4,8135.6, 134.7, 130.2, 129.9, 127.5, 126.52, 126.48, 121.8, 121.0, 117.7, 115.4, 103.1, 69.3, 64.2, 62.2, 62.1, 60.5, 56.0, 44.0, 42.3, 40.8, 40.5, 40.1, 38.6, 34.2, 32.1, 29.93, 29.87, 29.7, 29.5, 29.34, 29.28, 28.2, 27.8, 27.5, 27.4, 27.3, 25.0, 22.8, 21.7, 21.2, 19.8, 15.2, 14.4, 14.3, 13.3.

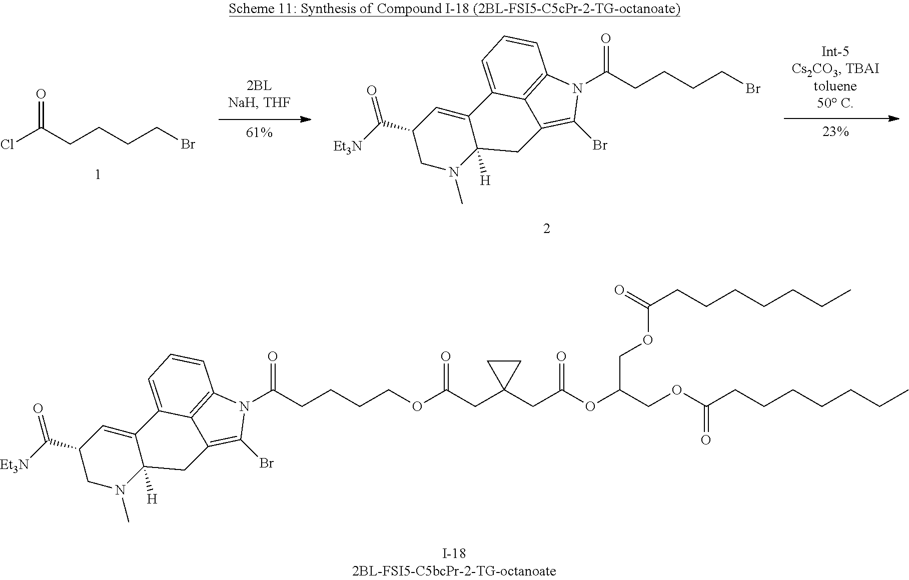

To a stirring solution of 2BL (150 mg, 373 μmol) in tetrahydrofuran (5 mL) was added sodium hydride (22.4 mg, 559 μmol, 60% w/w dispersion in mineral oil) and the mixture was stirred at RT for 10 minutes. To the solution was added 5-bromovaleryl chloride 1 (112 mg, 559 μmol, 74.9 μL) and the reaction left to stir at RT for 18 hours. The material was diluted with DCM (20 mL) and washed with water (10 mL), brine (10 mL), dried over MgSO₄, filtered and concentrated to give a crude oil. The material was purified by normal phase chromatography (Biotage Isolera, 12 g SiliaSep cartridge; eluent 0-30% (10% methanol in DCM) in DCM over 30 CVs) to afford 2 (150 mg, 61% yield) as a yellow oil.

UPLC6-MS: (BEH-C18 Short Base 2 to 95%) Rt=1.13 min., 86.2% (UV), MS (ESIpos): m/z=[M+H]+ 566.1, 568.1 79Br/81Br isotope pair.

¹H NMR (400 MHZ, CDCl₃): δ 7.97 (d, J=8.1 Hz, 1H), 7.37 (d, J=7.3 Hz, 1H), 7.29 (d, J=8.1 Hz, 1H), 6.39-6.35 (m, 1H), 4.06-3.85 (m, 1H), 3.58-3.29 (m, 7H), 3.28-3.17 (m, 2H), 3.09 (br dd, J=4.0, 10.6 Hz, 1H), 3.01-2.81 (m, 1H), 2.75-2.48 (m, 4H), 2.11-1.92 (m, 4H), 1.79 (br dd, J=2.0, 4.8 Hz, 1H), 1.30-1.22 (m, 3H), 1.17 (t, J=7.1 Hz, 3H).

To a solution of 2 (150 mg, 228 μmol) and Int-5 (111 mg, 229 μmol) in toluene (4 mL), cesium carbonate (223 mg, 684 μmol) and TBAI (43.0 mg, 114 μmol) were added at RT. The reaction mixture was heated to 50° C. and was stirred for 2 hours. The reaction mixture was allowed to cool to RT, diluted with ethyl acetate (25 mL), and washed with water (25 mL) and brine (25 mL). The organic layer was dried over sodium sulfate, filtered and concentrated to give a crude yellow oil. The material was purified by normal phase chromatography (Biotage Isolera, 25 g SiliaSep cartridge; eluent 0-80% ethyl acetate (+0.5% Et₃N) in heptane (+0.5% Et₃N) over 30 CVs) to afford 2BL-FSI5-C5bcPr-2-TG-octanoate, Compound I-18 (50.0 mg, 23% yield) as a yellow oil.

UPLC4-MS: (XB BEH 300 C4 20 to 95%): Rt=5.38 min., 89.8% (UV), 100.0% (ELSD). MS (ESIpos): [M+H]+ m/z=968.8, 970.8 79Br/81Br isotope pair.

¹H NMR (400 MHZ, CDCl₃): δ 7.95 (d, J=8.1 Hz, 1H), 7.35 (d, J=7.5 Hz, 1H), 7.26 (t, J=6.2 Hz, 1H), 6.35 (s, 1H), 5.29-5.20 (m, 1H), 4.29 (dd, J=4.4, 11.9 Hz, 2H), 4.16-4.07 (m, 4H), 3.89 (br s, 1H), 3.48-3.31 (m, 5H), 3.19 (br t, J=7.2 Hz, 3H), 3.05 (br dd, J=4.6, 11.1 Hz, 1H), 2.92-2.79 (m, 1H), 2.61 (s, 3H), 2.51-2.45 (m, 1H), 2.42 (s, 2H), 2.38 (s, 2H), 2.29 (t, J=7.6 Hz, 4H), 1.96-1.85 (m, 2H), 1.81-1.73 (m, 2H), 1.58 (quin, J=7.3 Hz, 4H), 1.32-1.19 (m, 19H), 1.15 (t, J=7.1 Hz, 3H), 0.85 (t, J=6.4 Hz, 6H), 0.53 (m, 4H).

¹³C NMR (100 MHZ, CDCl₃): δ 173.3, 172.3, 172.2, 171.33, 171.25, 135.5, 134.5, 127.3, 126.4, 121.7, 120.8, 117.6, 115.4, 103.1, 69.1, 64.0, 62.1, 62.0, 55.9, 43.8, 42.2, 41.07, 41.05, 40.5, 39.9, 38.6, 34.1, 31.7, 29.2, 29.0, 28.2, 27.6, 24.9, 22.7, 21.6, 15.1, 14.4, 14.2, 13.3, 12.3.

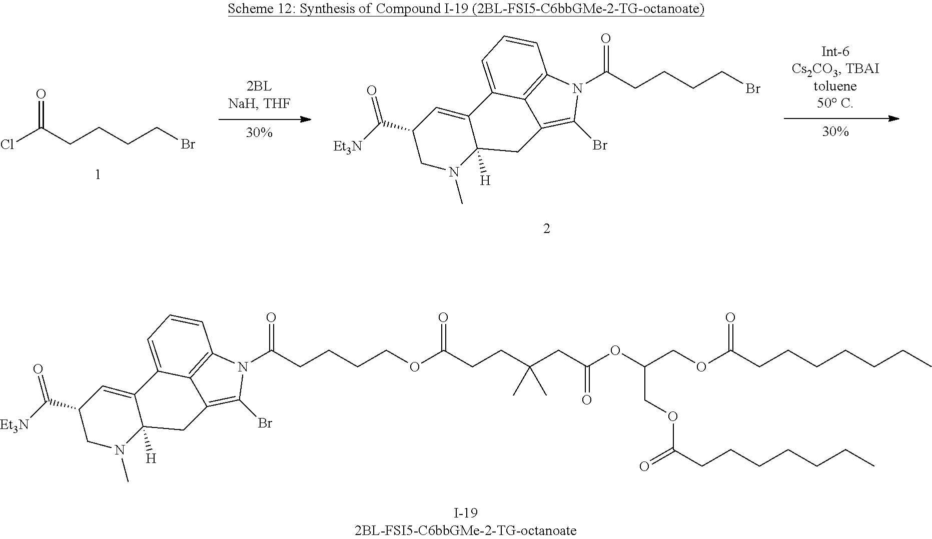

To a stirring solution of 2BL (600 mg, 1.49 mmol) in DMF (10 mL) at 0° C. under argon, was added NaH (89.5 mg, 2.24 mmol, 60% w/w dispersion in mineral oil). The mixture was stirred for 30 minutes while maintaining the temperature at 0° C. 5-bromovaleryl chloride (446 mg, 2.24 mmol, 299 μL) was then added, and the mixture was allowed to warm to RT and stir for 18 hours. The reaction was quenched with saturated aqueous NH₄Cl (50 mL), and the mixture was extracted with diethyl ether (3×50 mL). The combined organic layers were washed with brine (100 mL), dried over MgSO₄, filtered, and concentrated to give a crude oil. The material was purified by normal phase chromatography (Biotage Isolera, 40 g SiliaSep cartridge; eluent 0-10% methanol in DCM over 30 CVs) to afford 2 (505 mg, 30% yield) as a yellow oil.

UPLC6 (BEH C18 Long Neutral 20 to 95): Rt=2.73 min, 51.3% (UV). MS (ESIpos): m/z=566.0, 568.0 [M+H]+.

¹H NMR (400 MHZ, CDCl₃) δ 7.97 (d, J=8.2 Hz, 1H), 7.38 (d, J=7.6 Hz, 1H), 7.31-7.23 (m, 1H), 6.40-6.35 (m, 1H), 3.92 (br s, 1H), 3.61 (t, J=6.3 Hz, 1H), 3.56-3.31 (m, 4H), 3.21 (br t, J=6.2 Hz, 2H), 3.12 (br d, J=4.4 Hz, 1H), 2.97-2.85 (m, 1H), 2.63 (br s, 3H), 2.60-2.46 (m, 2H), 2.42-2.31 (m, 2H), 2.04-1.86 (m, 2H), 1.84-1.74 (m, 2H), 1.26 (t, J=7.1 Hz, 3H), 1.17 (t, J=7.1 Hz, 3H); product contained 5.74% w/w DCM, and an additional impurity, as seen by UPLC.

To a solution of Int-6 (147 mg, 294 μmol) and 2 (250 mg, 294 μmol) in toluene (20 mL), cesium carbonate (291 mg, 894 μmol) and TBAI (57.2 mg, 155 μmol) were added at RT. The reaction mixture was heated to 50° C. and was stirred for 3 hours. The reaction was quenched with a saturated aqueous solution of NH₄Cl (100 mL), and the mixture was extracted with ethyl acetate (3×20 mL). The combined organic layers were washed with brine (100 mL), dried over MgSO₄ and concentrated to give a crude residue. The material was purified by normal phase chromatography (Biotage Isolera, 40 g SiliaSep cartridge; eluent 0-10% methanol in DCM over 15 CVs) to afford 2BL-FSI5-C6bbGMe-2-TG-octanoate, Compound I-19 (88.0 mg, 30% yield) as a yellow oil.

UPLC4-MS (XB BEH300 C4 20 to 95%): Rt=5.58 min., 66.7% (UV), 93.2% (ELSD); MS(ESIpos): m/z=986.7, 987.8 [M+H]+. 79Br/81Br isotope pair.

¹H NMR (400 MHZ, CDCl₃): δ 7.95 (d, J=8.2 Hz, 1H), 7.36 (d, J=7.6 Hz, 1H), 7.26 (t, J=7.7 Hz, 1H), 6.35 (s, 1H), 5.29-5.23 (m, 1H), 4.31-4.24 (m, 2H), 4.17-4.08 (m, 4H), 3.88-3.82 (m, 1H), 3.52-3.32 (m, 6H), 3.24-3.12 (m, 3H), 3.07-2.99 (m, 1H), 2.88-2.80 (m, 1H), 2.59 (s, 3H), 2.32-2.26 (m, 6H), 2.21 (s, 2H), 1.99-1.85 (m, 2H), 1.85-1.74 (m, 2H), 1.70-1.55 (m, 6H), 1.30-1.22 (m, 19H), 1.16 (t, J=7.1 Hz, 3H), 0.99 (s, 6H), 0.89-0.83 (m, 6H); product contained a minor impurity.

¹³C NMR (101 MHZ, CDCl₃) δ 173.9, 173.4, 172.4, 171.4, 171.0, 135.5, 134.6, 127.4, 126.5, 121.7, 121.0, 117.7, 115.4, 103.1, 69.0, 64.2, 62.3, 62.0, 55.9, 45.7, 43.9, 42.2, 40.5, 40.0, 38.6, 36.9, 34.1, 33.1, 31.8, 29.6, 29.2, 29.0, 28.2, 27.7, 27.3, 27.0, 24.9, 22.7, 21.6, 15.1, 14.2, 13.3.

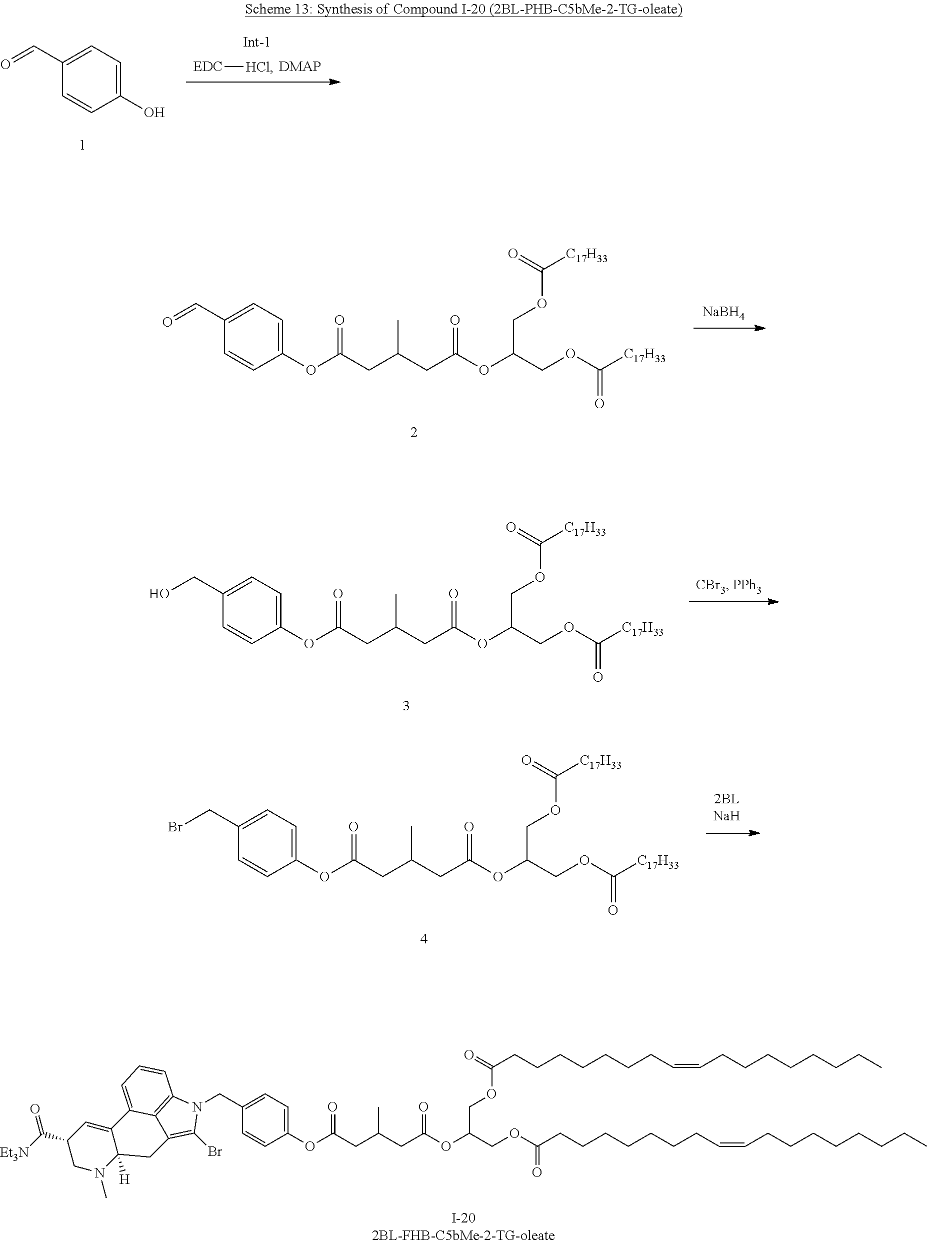

4-Hydroxybenzaldehyde 1 (277 mg, 2.27 mmol, 245 μL) and DMAP (555 mg, 4.55 mmol) was added to a solution of Int-1 (1.70 g, 2.27 mmol) and EDC-HCl (870 mg, 4.54 mmol) in DCM (30 mL), and the mixture was stirred at RT for about 18 hours. Material was purified by normal phase chromatography (BioTage Isolera, 40 g SiliaSep Cartridge) using a 0-30% ethyl acetate eluent in heptane over 20 CVs to afford (2) (1.41 g, 69% yield) as a colorless oil.

¹H NMR (400 MHZ, CDCl₃) δ=9.99 (s, 1H), 7.92 (d, J=7.7 Hz, 2H), 7.30-7.26 (m, 2H), 5.39-5.26 (m, 5H), 4.36-4.27 (m, 2H), 4.15 (ddd, J=1.1, 6.0, 12.0 Hz, 2H), 2.79-2.66 (m, 1H), 2.65-2.45 (m, 3H), 2.42-2.34 (m, 1H), 2.30 (dt, J=1.5, 7.5 Hz, 4H), 2.05-1.97 (m, 8H), 1.62-1.56 (m, 4H), 1.40-1.20 (m, 40H), 1.14 (d, J=4 Hz, 3H), 0.88 (t, J=8 Hz, 6H);

¹³C NMR (101 MHZ, CDCl₃) δ=191.0, 173.4, 171.3, 170.2, 155.4, 134.2, 131.4, 130.2, 129.8, 122.5, 69.5, 62.2, 40.7, 34.1, 32.1, 29.91, 29.85, 29.7, 29.5, 29.31, 29.26, 29.2, 27.5, 27.4, 27.3, 25.0, 22.8, 19.9, 14.3.

Sodium borohydride (60.2 mg, 1.59 mmol) was added portion wise to a solution of 2 (1.4 g, 1.56 mmol) in THF (14 mL) and MeOH (14 mL) and the reaction stirred at 0° C. for 1 hour. A. The mixture was diluted with ethyl acetate (100 mL), water (100 mL), brine (100 mL), dried with sodium sulfate and concentrated. The material was purified by normal phase chromatography (Biotage Isolera, 40 g SiliaSep cartridge, eluent 0-40% ethyl acetate in heptane over 16 CVs) to yield 3 (1.14 g, 81% yield).

¹H NMR (400 MHZ, CDCl₃) δ=7.38 (d, J=8.4 Hz, 2H), 7.07 (d, J=8.4 Hz, 2H), 5.42-5.23 (m, 5H), 4.69 (s, 2H), 4.31 (dt, J=4.2, 11.9 Hz, 2H), 4.14 (ddd, J=2.2, 6.0, 12.0 Hz, 2H), 2.70-2.54 (m, 2H), 2.54-2.45 (m, 2H), 2.41-2.27 (m, 5H), 2.08-1.94 (m, 8H), 1.70-1.53 (m, 4H), 1.28 (br d, J=9.3 Hz, 40H), 1.16-1.11 (m, 3H), 0.88 (t, J=6.8 Hz, 6H); Exchangeable proton not observed.

¹³C NMR (101 MHz, CDCl₃) δ=173.4, 171.4, 170.9, 150.1, 138.7, 130.2, 129.9, 128.2, 121.8, 69.4, 64.9, 62.2, 40.7, 34.1, 32.0, 29.9, 29.8, 29.7, 29.5, 29.31, 29.26, 29.2, 27.6, 27.4, 27.3, 25.0, 22.8, 19.9, 14.3.

A solution of 3 (1.14 g, 1.27 mmol), CBr₄ (1.05 g, 3.17 mmol), and PPh₃ (342 mg, 1.30 mmol) in dichloromethane (30 mL) was stirred at RT for 18 hours. The reaction was concentrated and the residue was purified by normal phase chromatography (Biotage Isolera, 40 g SiliaSep cartridge) using an eluent of 0-30% ethyl acetate in heptane over 12 CVs to yield 4 (790 mg, 65% yield) as a colorless oil.

¹H NMR (400 MHZ, CDCl₃) δ=7.40 (d, J=7.6 Hz, 2H), 7.06 (d, J=7.6 Hz, 2H), 5.39-5.27 (m, 5H), 4.48 (s, 2H), 4.37-4.27 (m, 2H), 4.14 (dd, J=6.0, 12.0 Hz, 2H), 2.69-2.61 (m, 1H), 2.61-2.53 (m, 1H), 2.53-2.45 (m, 2H), 2.41-2.27 (m, 5H), 2.14-1.88 (m, 8H), 1.60 (br t, J=7.0 Hz, 4H), 1.40-1.23 (m, 40H), 1.13 (d, J=6.5 Hz, 3H), 0.95-0.81 (m, 6H);

¹³C NMR (101 MHz, CDCl₃) δ=173.4, 171.4, 170.6, 150.6, 135.5, 130.3, 130.1, 129.8, 122.0, 69.4, 62.2, 40.7, 34.1, 32.7, 32.0, 29.9, 29.82, 29.78, 29.6, 29.5, 29.4, 29.3, 29.23, 29.20, 27.6, 27.34, 27.29, 24.9, 22.8, 19.8, 14.2.

TBAI (28.2 mg, 76.4 μmol) and tert-BuOK (25.1 mg, 224 μmol) was added to a solution of 2BL (60 mg, 149 μmol) in THF (3 mL) and stirred at RT for 10 minutes. 4 (123 mg, 134 μmol) was added and the mixture stirred for 2 hours at RT. The reaction as diluted with ethyl acetate (30 mL), washed with water (20 mL), and brine (30 mL). The organic layer was dried over MgSO₄, filtered, and concentrated. The material was purified by normal phase chromatography (Biotage Isolera, 12 g SiliaSep cartridge) using an eluent of 0-100% ethyl acetate in heptane to yield 2BL-PHB-C5bMe-2-TG-oleate, Compound I-20, as a yellow oil (80 mg, 38% yield).

¹H NMR (400 MHZ, CDCl₃) δ=7.23-7.17 (m, 1H), 7.15-7.06 (m, 3H), 7.06-7.02 (m, 1H), 6.99 (d, J=8.6 Hz, 2H), 6.36 (s, 1H), 5.40-5.24 (m, 7H), 4.36-4.25 (m, 2H), 4.18-4.09 (m, 2H), 3.90 (br s, 1H), 3.52-3.38 (m, 5H), 3.26 (br s, 1H), 3.07 (br d, J=7.2 Hz, 1H), 2.92 (br d, J=10.1 Hz, 1H), 2.68-2.43 (m, 8H), 2.37-2.23 (m, 5H), 2.08-1.93 (m, 8H), 1.64-1.53 (m, 4H), 1.37-1.21 (m, 43H), 1.17 (t, J=7.1 Hz, 3H), 1.10 (d, J=6.5 Hz, 3H), 0.92-0.82 (m, 6H);

¹³C NMR (101 MHZ, CDCl₃) δ=173.4, 171.4, 170.7, 150.0, 135.1, 135.0, 130.2, 129.9, 127.8, 126.0, 123.5, 121.9, 120.8, 113.4, 108.5, 69.4, 62.8, 62.2, 56.1, 53.6, 47.8, 44.1, 42.2, 40.7, 40.4, 34.1, 32.0, 29.9, 29.8, 29.7, 29.5, 29.30, 29.25, 29.2, 27.6, 27.4, 27.3, 25.0, 22.8, 19.8, 15.1, 14.3, 13.3.

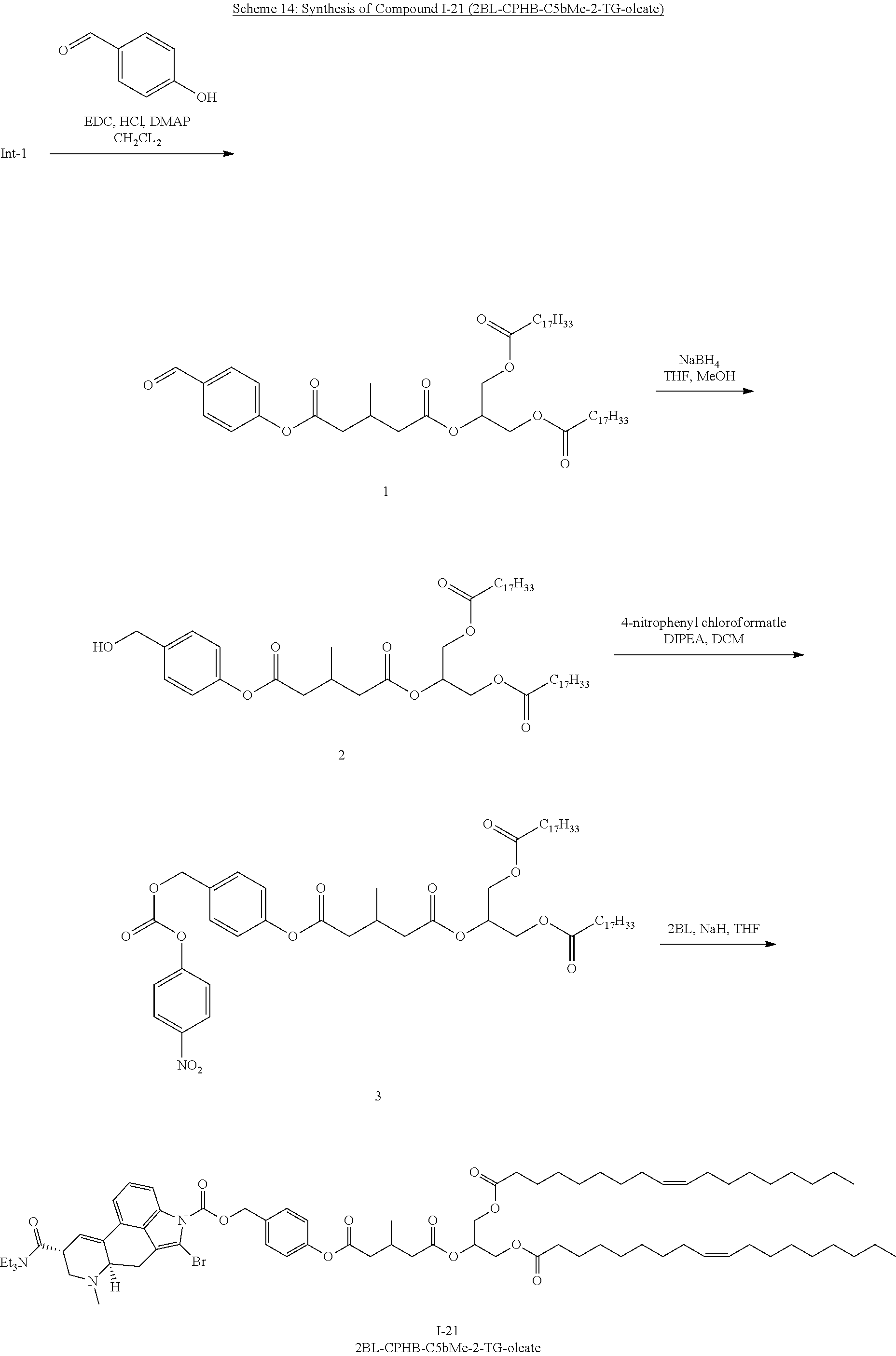

N-ethyl-N′-(3-dimethylaminopropyl) carbodiimide hydrochloride (1.02 g, 5.34 mmol) and 4-dimethylaminopyridine (652 mg, 5.34 mmol) was added to a stirred solution 4-hydroxybenzaldehyde (326 mg, 2.67 mmol) and Int-1 (2 g, 2.67 mmol) in dichloromethane (40 mL). The mixture was stirred at RT for 18 hours, concentrated under reduced pressure, and purified by normal phase chromatography (Biotage Isolera, 80 g SiliaSep cartridge) using a 0-20% ethyl acetate in heptane eluent to afford 1 (1.82 g, 77% yield).

¹H NMR (400 MHZ, CDCl₃): δ [ppm]=10.00 (s, 1H), 7.92 (d, J=8.6 Hz, 2H), 7.28 (d, J=8.6 Hz, 2H), 5.41-5.25 (m, 5H), 4.33 (ddd, J=1.5, 4.1, 11.9 Hz, 2H), 4.15 (dd, J=6.0, 11.9 Hz, 2H), 2.75-2.68 (m, 1H), 2.65-2.45 (m, 3H), 2.43-2.35 (m, 1H), 2.31 (t, J=7.5 Hz, 4H), 2.04-1.96 (m, 8H), 1.63-1.57 (m, 4H), 1.28 (br d, J=9.3 Hz, 40H), 1.15 (d, J=6.4 Hz, 3H), 0.88 (t, J=6.8 Hz, 6H).

¹³C NMR (100 MHZ, CDCl₃): δ [ppm]=191.0, 173.4, 171.3, 170.2, 155.4, 134.2, 131.4, 130.2, 129.9, 122.5, 69.5, 62.2, 40.67, 40.65, 34.2, 32.1, 29.91, 29.85, 29.7, 29.5, 29.32, 29.26, 29.24, 27.5, 27.4, 27.3, 25.0, 22.8, 19.9, 14.3.

Sodium borohydride (85 mg, 2.25 mmol) was added portion wise to a stirring solution of 1 (1.82 g, 2.13 mmol) in THF (20 mL) and methanol (20 mL) and stirred at RT for 3 hours. The mixture was diluted with water (80 mL) and extracted into ethyl acetate (2×70 mL). The combined organic extracts were washed with brine (80 mL), dried over sodium sulfate, filtered, and concentrated. Material was purified by normal phase chromatography (Biotage Isolera, 80 g SiliaSep cartridge) using an eluenet of 0-100% ethyl acetate in heptane to afford 2 (790 mg, 41% yield) as a colorless oil.

¹H NMR (400 MHZ, CDCl₃): δ [ppm]=7.38 (d, J=8.6 Hz, 2H), 7.08-7.05 (m, 2H), 5.39-5.24 (m, 5H), 4.69 (d, J=5.9 Hz, 2H), 4.31 (td, J=4.2, 11.9 Hz, 2H), 4.19-4.10 (m, 2H), 2.71-2.54 (m, 2H), 2.54-2.44 (m, 2H), 2.42-2.33 (m, 1H), 2.33-2.27 (m, 4H), 2.05-1.96 (m, 8H), 1.66-1.56 (m, 4H), 1.36-1.21 (m, 40H), 1.14 (d, J=6.5 Hz, 3H), 0.88 (t, J=6.7 Hz, 6H); Exchangeable hydroxyl proton not observed.

Nitrophenyl chloroformate (47.1 mg, 234 μmol) was added to a stirring solution of 2 (213 mg, 234 μmol) in DCM (4 mL), followed by a solution of N,N-diisopropylethylamine (167 μL, 959 μmol) at 0° C. The reaction was warmed to RT and stirred for 18 hours. The mixture was diluted with water (10 mL) and DCM (6 mL). The organic layer was extracted, concentrated, and purified by normal phase chromatography (Biotage Isolera, 12 g SiliaSep cartridge) using 0-20% ethyl acetate in heptane to afford 3 (123 mg, 32% yield) as a colorless oil.

¹H NMR (400 MHZ, CDCl₃): δ [ppm]=8.28 (d, J=9.3 Hz, 2H), 7.47 (d, J=8.6 Hz, 2H), 7.43-7.35 (m, 2H), 7.16-7.04 (m, 2H), 5.40-5.25 (m, 5H), 5.14 (s, 2H), 4.37-4.27 (m, 2H), 4.14 (br dd, J=6.0, 11.9 Hz, 2H), 2.73-2.63 (m, 1H), 2.63-2.54 (m, 1H), 2.54-2.44 (m, 2H), 2.42-2.34 (m, 1H), 2.34-2.28 (m, 4H), 2.06-1.94 (m, 8H), 1.60 (br t, J=6.6 Hz, 4H), 1.36-1.20 (m, 40H), 1.13 (dd, J=4.3, 6.5 Hz, 3H), 0.87 (t, J=6.5 Hz, 6H).

Sodium hydride (5.79 mg, 145 μmol, 60% dispersion in mineral oil) was added to a solution of 2BL (48.5 mg, 121 μmol) in THF (3 mL) and stirred for 30 minutes. A solution of 3 dissolved in THF (1 mL0 was added to the mixture. The mixture was warmed to RT and stirred for 18 hours. The mixture was diluted with ethyl acetate (15 mL) and washed with water (2×15 mL) and brine (2×15 mL). The organic layer was dried over sodium sulfate, filtered, and concentrated to a yellow oil. Material was purified by normal phase chromatography (Biotage Isolera, 12 g SiliaSep cartridge) using an eluent of 0-100% ethyl acetate+0.5% triethylamine in heptane to afford 2BL-CPHB-C5bMe-2-TG-oleate, Compound I-21 (60 mg, 32% yield) as a dark-yellow gum.

### Example 3: Metabolism Identification (MetID) Assay in Human Hepatocytes, Rat Hepatocytes, and Human Liver Microsomes

To better understand the clearance mechanisms and biotransformation capabilities, parse the presence of potential active or reactive metabolites, and observe the disproportionate or unique metabolites of bromolysergide (2-bromo-LSD), MetID assays were conducted. Samples employed for MetID included human hepatocytes, rat hepatocytes, and the human liver microsomes S9 fraction. Hepatocytes were prepared by thawing cryopreserved cells in media (in VitroGRO KHB Buffer, IVT) to a concentration of 1,000,000 cells/mL. After pre-incubation for 5 minutes at 37 degrees Celsius, the hepatocytes were incubated with bromolysergide (2-bromo-LSD) (final concentration 10 μM with <0.2% organic) for 3 hours also at 37 degrees Celsius. Next, 100 μL of the incubation mix was removed after the incubation and quenched with 100 μL acetonitrile before vortexing and centrifuging at 13,000 rpm for 5 minutes total. 175 μL of supernatant was taken after centrifuging, added to a 96-well plate, and dried using nitrogen to reach 100 μL sample for liquid chromatography-high resolution mass spectrometry.

Liver microsome incubations were conducted similarly. 1 mg/mL of cryopreserved liver microsome S9 fraction was incubated in a phosphate buffer (PH 7.4, 100 mM with 3 mM of MgCl₂) at 37° C. After 5 minutes, 1 mM NADPH was added to begin the reaction and the microsomes were incubated with bromolysergide (2-bromo-LSD) for one hour, with the final concentration of bromolysergide (2-bromo-LSD) being 10 μM with <0.2% organic. Next, 100 μL of the incubation mix was removed after the incubation and quenched with 100 μL acetonitrile before vortexing and centrifuging at 10,000 rpm for 5 minutes total. 170 μL of supernatant was taken after centrifuging and dried down to reach a 100 μL sample for ultra-performance liquid chromatography-high resolution mass spectrometry.

In both types of incubation, 5.0 μM verapamil was used as a positive control, while the negative control consisted of only incubation buffer. LC/MS data were processed manually with assistance from programs Xcalibur, Freestyle, and Compound Discoverer. The results summarized below in Table 2.

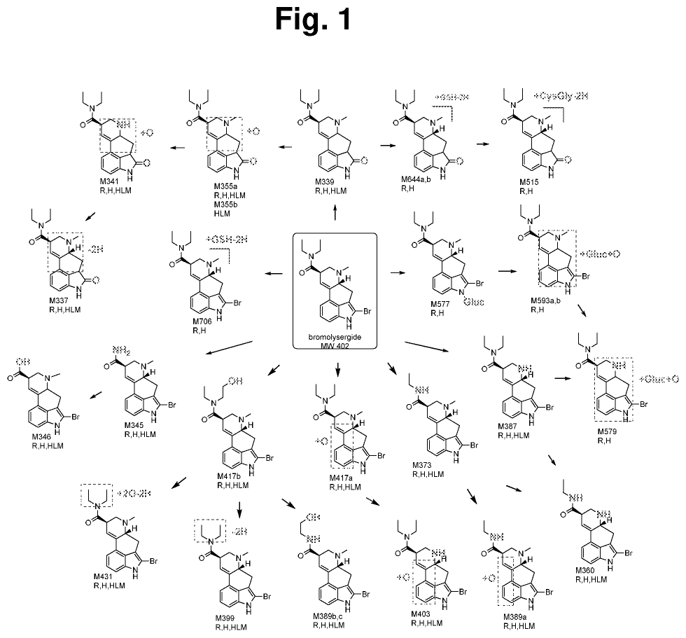

The MetID results determined that lysergic acid diethylamide (LSD) is not a major metabolite (FIG. 1, Table 2). MetID was also conducted because a major metabolite of bromolysergide (2-bromo-LSD) could be a strong agonist of the 5-HT_{2B} receptor, which could lead to fibrotic side effects with chronic dosing. None of the major metabolites identified in the MetID results seem likely to activate the 5-HT_{2B} receptor due to the stability of the bromine atom. The few metabolites that were de-brominated were rapidly oxidated, saturating the ring structure. Therefore, these metabolites are also unlikely to activate the 5-HT_{2B} receptor.

TABLE 2
Metabolites of bromolysergide identified in human liver microsomes
(Human_LM), rat hepatocytes (Rat), and human hepatocytes (Human).

| | RT | Observed | Accuracy | | % of Total | | |
|---|---|---|---|---|---|---|---|
| Identifier | (min) | (m/z) | (ppm) | Biotransformation | Human_LM | Rat | Human |
| M341 | 4.23 | 342.1810 | −0.7 | —CH2—Br + OH + O | 0.1 | 0.3 | 0.04 |
| M644a | 4.50 | 645.2698 | −0.4 | —Br + GSH + O—2H | — | 2.8 | 0.79 |
| M644b | 4.58 | 645.2698 | −0.4 | —Br + GSH + O—2H | — | 30.9 | 5.2 |
| M355a | 4.56 | 356.1968 | −0.2 | —Br + OH + O | 32.3 | 11.6 | 4.3 |
| M389 | 4.74 | 390.0812 | 0.0 | —C2H4 + O | 0.6 | 0.1 | 0.03 |
| M355b | 4.84 | 356.1968 | −0.2 | —Br + OH + O | 1.2 | <0.01 | — |
| M579 | 4.92 | 580.1295 | 1.1 | —CH2 + Gluc + O | — | 3.3 | 0.1 |
| M593a | 5.00 | 594.1446 | 0.1 | +O + Gluc | — | 1.7 | 1.4 |
| M389b | 5.11 | 390.0812 | 0.0 | —C2H4 + O | 0.5 | 0.01 | 0.03 |
| M345 | 5.14 | 346.0548 | −0.6 | —C4H8 | 0.2 | 0.1 | 0.4 |
| M577 | 5.21 | 578.1498 | 0.2 | +Gluc | — | 0.2 | 0.1 |
| M593b | 5.35 | 594.1445 | −0.1 | +Gluc + O | — | 7.0 | 0.8 |
| M339 | 5.55 | 340.2020 | 0.2 | —Br + OH | 4.4 | 3.9 | 4.8 |
| M389c | 5.78 | 390.0812 | 0.0 | —C2H4 + O | 0.5 | 0.01 | 0.02 |
| M515 | 5.79 | 516.2278 | 0.6 | —Br + CysGly-2H | — | 0.2 | 5.3 |
| M706 | 5.81 | 707.1851 | −0.8 | +GSH-2H | — | 2.9 | 0.4 |
| M346 | 5.88 | 347.0388 | −0.5 | amide hydrolysis | 0.9 | 0.5 | 14.3 |
| M359 | 6.14 | 360.0706 | 0.0 | —C3H6 | 1.2 | 0.7 | 1.0 |
| M403 | 6.16 | 404.0968 | 0.0 | —CH2 + O | 0.01 | 0.4 | 0.01 |
| M417a | 6.22 | 418.1130 | 1.4 | +O | 2.3 | 1.1 | 0.5 |
| M373 | 6.40 | 374.0868 | 1.5 | —C2H4 | 19.6 | 7.7 | 10.3 |
| M417b | 6.53 | 418.1121 | −0.9 | +O | 1.2 | 1.3 | 1.5 |
| M431 | 6.64 | 432.0921 | 0.9 | +2O—2H | 0.4 | 0.2 | 1.1 |
| M387 | 7.98 | 388.1009 | −2.7 | —CH2 | 3.0 | 2.5 | 3.0 |
| Bromolyserside | 8.10 | 402.1179 | 0.8 | parent | 30.0 | 18.5 | 41.3 |
| M399 | 8.882 | 400.1028 | 2.4 | —H2 | 1.5 | 0.5 | 3.2 |
| M337 | 9.63 | 338.1857 | −1.8 | —Br + OH—2H | 0.03 | 1.7 | 0.1 |

### Example 4: In vitro Hydrolysis of Lipid Prodrugs of 2-Bromo-LSD

The selectivity of prodrug hydrolysis to recombinant human esterases, rhCES1, rhCES2, and rhBChE, was evaluated. For each recombinant human esterase, 50 μM of prodrug was incubated in 10 mg/mL bovine serum albumin (BSA), 300 units/mL of lipoprotein lipase, and 0.5 μg/mL of a recombinant human esterase. Reactions were initiated via the addition of prodrug. After a 30-minute incubation at 37° C., reactions were quenched with the addition of cold acetonitrile (3:1 acetonitrile to reaction volume) spiked with an internal standard(s). Samples are vortexed and centrifuged and the supernatant is transferred and diluted in 0.1% formic acid (1:3 supernatant to 0.1% formic acid). Samples are analyzed by LC-MS, and the percent of 2-bromo-LSD is calculated using a standard curve of 2-bromo-LSD in BSA (10 mg/mL in PBS).

To determine the rate of 2-bromo-LSD release catalyzed by each esterase, reactions can be prepared as described above. The mixture is incubated at 37° C. and 50 μL aliquots removed from the incubation at 5, 10, 15, 20, and 30 minutes. For each aliquot, the reaction is quenched with the addition of 150 μL of cold acetonitrile spiked with d3-LSD (40 ng/ml). Samples are vortexed and centrifuged and the supernatant is transferred and diluted in 0.1% formic acid (1:3 supernatant to 0.1% formic acid). Samples are analyzed by LC-MS, and the percent of 2-bromo-LSD is calculated using a standard curve of 2-bromo-LSD in BSA (10 mg/mL in PBS).

### Example 5: In vitro Stability of Lipid Prodrugs of 2-Bromo-LSD

The stability of lipid prodrugs of 2-bromo-LSD was evaluated in the presence or absence of recombinant human monoacylglyceride lipase (rhMAGL). 50 UM of prodrug was incubated in 10 mg/mL BSA supplemented with lipoprotein lipase (300 units/mL) and rhMAGL (0-0.5 μg/mL). The concentration of MAGL may be adjusted based on observed activity. Reactions were initiated via the addition of prodrug and incubated at 37° C. for 180 minutes. Aliquots were removed at 30, 60, 120, and 180 minutes and the reaction quenched with the addition of cold acetonitrile spiked with internal standard(s) (1:3 sample to acetonitrile). Samples were vortexed and centrifuged and the supernatant transferred and diluted in 0.1% formic acid water (1:3 supernatant to 0.1% formic acid water). Samples were analyzed via LC-MS. Monoglyceride stability was calculated using the following equations:

LPL MG Ratio=MG Peak Area at 180 minutes for LPL only/MG Peak area at 30 minutes for LPL only

rhMAGLMG Ratio=MG Peak Area at 180 minutes for LPLand rhMAGL/MG Peak Area at 30 minutes for LPLand rhMAGL

MG Stability=((rhMAGLMG Ratio−(1−LPL MG Ratio))*10

Monoglyceride stability of the lipid prodrugs is shown in Table 3. Stability is calculated on a scale of −10 to 10, with 10 representing 100% monoglyceride remaining (i.e., highest stability) and −10 representing 100% monoglyceride loss (i.e., lowest stability). Monoglyceride stability is represented by the following categories, wherein A is a stability score of 5 to 10, B is a stability score of 0 to 5, C is a stability score of −5 to 0, and D is a stability score of −5 to −10.

TABLE 3
Monoacylglyceride Stability of Lipid Prodrugs

| | | Monoacylglyceride |
|---|---|---|
| ID | Compound | Stability |
| I-22 | 2BL-FSI4-C6bbGMe-2-TG-octanoate | A |
| I-23 | 2BL-CASI-C6bbGMe-2-TG-octanoate | A |
| I-15′ | 2BL-FSI4-C5bcPr-2-TG-octanoate | A |
| I-9′ | 2BL-CASI-C5bcPr-2-TG-octanoate | A |
| I-21 | 2BL-CPHB-C5bMe-2-TG-oleate | A |
| I-24 | 2BL-C10bMe-2-TG-oleate | A |
| I-19 | 2BL-FSI5-C6bbGMe-2-TG-octanoate | A |
| I-9 | 2BL-CASI-C5bbGMe-2-TG-octanoate | A |
| I-15 | 2BL-FSI4-C5bbGMe-2-TG-octanoate | B |
| I-16 | 2BL-FSI5-C5bbGMe-2-TG-octanoate | A |
| I-14 | 2BL-FSI4-C5bMe-2-TG-oleate | A |
| I-25 | 2BL-ASI-C5bcPr-2-TG-octanoate | D |
| I-26 | 2BL-C5bcPr-2-TG-octanoate | A |
| I-18 | 2BL-FSI5-C5bcPr-2-TG-octanoate | A |
| I-27 | 2BL-ASI-C6bbGMe-2-TG-octanoate | D |
| I-20 | 2BL-PHB-C5bMe-2-TG-oleate | A |
| I-7 | 2BL-CASI-C5bMe-2-TG-octanoate | A |
| I-28 | 2BL-ASI-C5bbGMe-2-TG-octanoate | D |
| I-3 | 2BL-C12b′bMe-2-TG-oleate | B |
| I-2 | 2BL-C8bMe-2-TG-oleate | A |
| I-6 | 2BL-ASI-C12bMe-2-TG-oleate | C |
| I-17 | 2BL-FSI5-C5bMe-2-TG-oleate | A |
| I-4 | 2BL-ASI-C5bMe-2-TG-oleate | D |
| I-5 | 2BL-ASI-C8bMe-2-TG-oleate | C |
| I-1 | 2BL-C5bMe-2-TG-oleate | A |

### Example 6: In Vitro Release of 2-Bromo-LSD from Lipid Prodrugs in Plasma

The release of 2-bromo-LSD in plasma supplemented with lipoprotein lipase was determined using an in vitro assay. Lipoprotein lipase (LPL) is a key enzyme for hydrolysis of lipoprotein associated triglycerides in systemic circulation and is expected to be involved in the lipolysis of 2-bromo-LSD from the triglyceride backbone in plasma, i.e., the release of free 2-bromo-LSD.

40 μM of a prodrug is incubated with human plasma containing 200-12,000 units/mL of LPL (LPL concentration is determined experimentally based on the units needed to generate maximum monoglyceride concentration after the reaction is initiated). Samples are incubated for 0-180 minutes at 37° C. Reaction is quenched with MeCN spiked with internal standard(s), vortexed, and centrifuged at 4000×g for 10 minutes to precipitate proteins. The supernatant is analyzed by LC-MS/MS and potential hydrolysis products are detected, namely the monoglyceride form, the acid form, and the free 2-bromo-LSD. The concentration of free 2-bromo-LSD is calculated using a standard curve of 2-bromo-LSD. The percentage of free 2-bromo-LSD present for the tested prodrugs is shown in Table 4, with +++ representing a percentage of 2-bromo-LSD greater than 25%, indicative of sufficient levels of 2-bromo-LSD released from the prodrug, ++ representing a percentage release of 2-bromo-LSD between 5-25%, and + representing a percentage of 2-bromo-LSD of less than 5%.

TABLE 4
Percentage Release of 2-Bromo-LSD in Human Plasma

| | | % 2-Br-LSD present |
|---|---|---|
| ID | Compound | at end of experiment |
| I-22 | 2BL-FSI4-C6bbGMe-2-TG-octanoate | +++ |
| I-23 | 2BL-CASI-C6bbGMe-2-TG-octanoate | +++ |
| I-15′ | 2BL-FSI4-C5bcPr-2-TG-octanoate | +++ |
| I-9′ | 2BL-CASI-C5bcPr-2-TG-octanoate | +++ |
| I-21 | 2BL-CPHB-C5bMe-2-TG-oleate | +++ |
| I-24 | 2BL-C10bMe-2-TG-oleate | +++ |
| I-19 | 2BL-FSI5-C6bbGMe-2-TG-octanoate | +++ |
| I-9 | 2BL-CASI-C5bbGMe-2-TG-octanoate | +++ |
| I-15 | 2BL-FSI4-C5bbGMe-2-TG-octanoate | +++ |
| I-16 | 2BL-FSI5-C5bbGMe-2-TG-octanoate | +++ |
| I-14 | 2BL-FSI4-C5bMe-2-TG-oleate | +++ |
| I-25 | 2BL-ASI-C5bcPr-2-TG-octanoate | +++ |
| I-26 | 2BL-C5bcPr-2-TG-octanoate | +++ |
| I-18 | 2BL-FSI5-C5bcPr-2-TG-octanoate | +++ |
| I-27 | 2BL-ASI-C6bbGMe-2-TG-octanoate | +++ |
| I-20 | 2BL-PHB-C5bMe-2-TG-oleate | + |
| I-7 | 2BL-CASI-C5bMe-2-TG-octanoate | +++ |
| I-28 | 2BL-ASI-C5bbGMe-2-TG-octanoate | ++ |
| I-3 | 2BL-C12b′bMe-2-TG-oleate | ++ |
| I-2 | 2BL-C8bMe-2-TG-oleate | +++ |
| I-6 | 2BL-ASI-C12bMe-2-TG-oleate | ++ |
| I-17 | 2BL-FSI5-C5bMe-2-TG-oleate | +++ |
| I-4 | 2BL-ASI-C5bMe-2-TG-oleate | +++ |
| I-5 | 2BL-ASI-C8bMe-2-TG-oleate | ++ |
| I-1 | 2BL-C5bMe-2-TG-oleate | + |

### Example 7: In vitro Release of 2-Bromo-LSD in Whole Blood

The release of free 2-bromo-LSD from the prodrug backbone was evaluated in whole blood supplemented with lipoprotein lipase. Lipoprotein lipase (LPL) is a key enzyme for hydrolysis of lipoprotein associated triglycerides in systemic circulation and is expected to be involved in the lipolysis of 2-bromo-LSD from the triglyceride backbone in plasma, i.e., the release of free 2-bromo-LSD.

Prodrug stock solutions were prepared at a 10 mM concentration in 7:3 MeCN: IPA. To start hydrolysis, human or NHP whole blood (stored at 4° C.) spiked with LPL (6000 IU/mL) and ketoprofen (400 ng/ml) was added to human or NHP plasma containing 80 μM of prodrug. Samples were incubated at RT with end over end mixing. Aliquots were taken from each reaction at 5, 30, 60, and 120 minutes, centrifuged at 2000×g for 10 minutes to collect plasma. 30 μL of plasma was added to 300 μL of organic solvent (1:1:1:0.1 of IPA: MeOH: MeCN: FA) containing internal standard(s). The mixtures were vortexed and centrifuged at 4000×g for 10 minutes to precipitate proteins. The supernatant was then analyzed by HPLC-MS/MS for potential hydrolysis products, including the monoglyceride form of the prodrug, the acid form of the prodrug, and free 2-bromo-LSD.

### Example 8: Pharmacokinetics of 2-Bromo-LSD Prodrugs in Rats

The systemic plasma exposure of 2-bromo-LSD resulting from oral administration of lipid prodrugs was determined in rats. Lipid based formulations of lipid prodrugs or control compounds were prepared as described in WO 2016/023082, WO 2017/041139, and Trevaskis N L et al., Pharmaceutical Research (2005) 22 (11): 1863-1870, incorporated herein by reference.

1 or 2 mg of 2-bromo-LSD or a prodrug, 40 mg of oleic acid, and 25 mg of polysorbate 80 were mixed in a glass vial and incubated at 37° C. for at least one hour and up to 18 hours to equilibrate. An aqueous phase of 2 mL phosphate buffered saline (PBS, pH 7.4) was added to the vial. The formulation was emulsified by ultrasonication using a probe tip sonicator for 2 minutes at RT in bursts of 10 seconds (10 seconds on, 10 seconds off). The temperature of the vial was maintained below 40° C. Formulation concentrations were verified by HPLC-MS or HPLC-UV.

As a control, 2-bromo-LSD was dissolved in DMSO and diluted 10-fold in aqueous 20% hydroxypropyl-beta-cyclodextrin (HPCD) solution for intravenous (IV) or oral (PO) administration.

Male Sprague-Dawley rats (220-320 g) were fasted overnight with free access to water. The next morning, rats were administered either prodrug formulation or 2-bromo-LSD control formulation via oral administration. As an additional control, 2-bromo-LSD was administered to rats via an IV bolus. Prodrugs were dosed at 4 mg/kg and normalized to 1 mg/kg of 2-bromo-LSD equivalent. After oral administration, blood samples (0.23 mL) were taken via venipuncture of the tail vein or other suitable site at −5 minutes up to 24 hours post-dosing. Blood samples were collected in KEDTA tubes prefilled with sufficient inhibitor solutions such that the final concentration of each inhibitor after sample collection was 40 μg/mL of orlistat and 100 μM of JZL-184 and GR148672X inhibitors. Plasma was separated by centrifugation and stored at −80° C. prior to analysis by HPLC-MS/MS. Table 5 provides the plasma exposure of 2-bromo-LSD in rats.

TABLE 5
Plasma Exposure of 2-Bromo-LSD in Rats

| | | Plasma | Plasma |
|---|---|---|---|
| | | AUC(0-last) | Cmax |
| | | (nmol · h/L) | (nmol/L) |
| ID | Compound | (±SD) | (±SD) |
| I-22 | 2BL-FSI4-C6bbGMe-2-TG- | 670 ± 120 | 230 ± 30 |
| | octanoate | | |
| I-23 | 2BL-CASI-C6bbGMe-2-TG- | 670 ± 150 | 260 ± 30 |
| | octanoate | | |
| I-21 | 2BL-CPHB-C5bMe-2-TG-oleate | 460 ± 90 | 180 ± 30 |
| I-24 | 2BL-C10bMe-2-TG-oleate | 800 ± 70 | 200 ± 40 |
| I-19 | 2BL-FSI5-C6bbGMe-2-TG- | 450 ± 90 | 160 ± 30 |
| | octanoate | | |
| I-9 | 2BL-CASI-C5bbGMe-2-TG- | 820 ± 170 | 180 ± 100 |
| | octanoate | | |
| I-16 | 2BL-FSI5-C5bbGMe-2-TG- | 390 ± 40 | 140 ± 30 |
| | octanoate | | |
| I-14 | 2BL-FSI4-C5bMe-2-TG-oleate | 530 ± 170 | 180 ± 70 |
| I-18 | 2BL-FSI5-C5bcPr-2-TG-octanoate | 440 ± 170 | 140 ± 70 |
| I-27 | 2BL-ASI-C6bbGMe-2-TG- | 210 ± 30 | 90 ± 20 |
| | octanoate | | |
| I-20 | 2BL-PHB-C5bMe-2-TG-oleate | 6 ± 3 | 3 ± 1 |
| I-7 | 2BL-CASI-C5bMe-2-TG-octanoate | 800 ± 210 | 200 ± 70 |
| I-28 | 2BL-ASI-C5bbGMe-2-TG- | 40 ± 8 | 10 ± 1 |
| | octanoate | | |
| I-2 | 2BL-C8bMe-2-TG-oleate | 550 ± 70 | 140 ± 10 |
| I-17 | 2BL-FSI5-C5bMe-2-TG-oleate | 510 ± 130 | 210 ± 40 |
| I-5 | 2BL-ASI-C8bMe-2-TG-oleate | 170 ± 10 | 80 ± 10 |
| 2BL | 2-Br-LSD (PO) | 920 ± 310 | 310 ± 50 |
| 2BL | 2-Br-LSD (IV) | 930 ± 100 | 890 ± 60 |

### Example 9: Pharmacokinetics of 2-Bromo-LSD Prodrugs in Dogs

The systemic plasma exposure of 2-bromo-LSD resulting from oral administration of lipid prodrugs is determined in dogs. Male beagle dogs will be fasted for 12 h up to 30 minutes prior to drug administration. For fed state studies, dogs will receive ~20 g of high fat dog food (containing ~34% fat), administered by hand, followed by 10 mL water, and 100 g standard canned dog food (~2.5% fat) 30 minutes prior to drug administration. Water will be available ad libitum throughout the study. Prodrugs may be prepared in a suitable SEDDS formulation, such as 25% w/w sesame oil, 27% w/w glycerol monooleate, and 48% polyethoxylated castor oil. Formulations can be filled in gelatin capsules or dispersed in water for oral gavage. As a control, 2-bromo-LSD was dissolved in DMSO and diluted 10-fold in aqueous 20% hydroxypropyl-beta-cyclodextrin (HPCD) solution.

Compounds may be administered orally by placing capsules posterior to the pharynx, closing the mouth, and stimulating swallowing. 50 mL of water will be administered orally via syringe. After oral administration, blood samples (~1.5 mL each) will be taken via venipuncture of the cephalic vein 5 minutes prior to administration up to 120 hours post-dosing. Plasma will be separated by centrifugation and aliquots of each plasma sample stored at −80° C. As a control, 2-bromo-LSD may be administered intravenously by either infusion (over 5 minutes) or bolus injection.

### Example 10: Pharmacokinetics of 2-Bromo-LSD Prodrugs in Non-Human Primates

The systemic plasma exposure of 2-bromo-LSD resulting from oral administration of lipid prodrugs was determined in non-human primates. Lipid prodrugs or 2-bromo-LSD were formulated in a SEDDS formulation of 25% w/w sesame oil, 27% w/w glycerol monooleate, and 48% polyethoxylated castor oil. Formulations can be filled in gelatin capsules or dispersed in water for oral gavage. As a control, 2-bromo-LSD was dissolved in DMSO and diluted 10-fold in aqueous 20% hydroxypropyl-beta-cyclodextrin (HPCD) solution.

Cynomolgus monkeys were fasted for 12 hours prior to administration. In fed-state studies, NHPs received 30 mL of an Ensure milkshake (available from Abbott) administered ~30 minutes before dosing followed by 10 mL water. Water was available ad libitum throughout the study. Formulations dispersed in water was orally administered to the NHPs by oral gavage and flushed with 50 mL of water after delivery. NHPs were dosed at 1 mg/kg equivalent of 2-bromo-LSD for the prodrugs. As a comparator control, 2-bromo-LSD was administered via oral gavage. To calculate bioavailability, 2-bromo-LSD was administered via IV bolus.

After oral administration, blood samples (0.6 mL) were taken via venipuncture of the cephalic vein or other suitable sample site at −5 minutes up to 120 hours post-dosing. Blood samples were collected in K2EDTA tubes prefilled with sufficient inhibitor solutions such that the final concentration of each inhibitor after sample collection was 40 g/mL of orlistat and 100 μM of JZL-184, GR148672X, and Rivastigmine inhibitors. Plasma was separated by centrifugation and stored at −80° C. prior to analysis. As a comparator control, 2-bromo-LSD was administered via oral gavage or IV infusion for the calculation of oral bioavailability.

Plasma samples were analyzed by LC-MS/MS to measure free and combined 2-bromo-LSD. To determine free 2-bromo-LSD, samples were extracted using protein precipitation and quantified against a standard curve of 2-bromo-LSD. As used herein, the term “combined 2-bromo-LSD” refers to 2-bromo-LSD detected as free 2-bromo-LSD, unhydrolyzed prodrug of 2-bromo-LSD, and hydrolysis intermediates of 2-bromo-LSD prodrugs. For the combined 2-bromo-LSD analysis, samples were diluted in IPA and incubated for 2 hours at RT following the addition of KOH (20 mg/mL). This incubation step hydrolyzes prodrug or prodrug intermediates to 2-bromo-LSD. After the reaction is neutralized, 2-bromo-LSD levels were quantified against a standard curve of the corresponding prodrug. Table 6 provides the plasma exposure of 2-bromo-LSD in male NHPs and Table 7 provides the plasma exposure of 2-bromo-LSD in female NHPs. Table 8 provides the combined levels of 2-bromo-LSD in male NHPs, and Table 9 provides the combined levels of 2-bromo-LSD in female NHPs.

FIG. 2A-2D demonstrate the systemic exposure of 2-bromo-LSD administered in an aqueous formulation (FIG. 2A), a SEDDS lipid formulation (FIG. 2B), or as prodrug of the present disclosure (FIGS. 2C and 2D).

TABLE 6
Plasma Exposure of 2-Bromo-LSD in Male NHPs

| | | | Plasma | Plasma |
|---|---|---|---|---|
| | Sample | | AUC(0-last) | Cmax |
| | Size | | (ng · h/mL) | (ng/mL) |
| ID | (N) | Compound | (±SD) | (±SD) |
| I-22 | 3 | 2BL-FSI4-C6bbGMe-2-TG-octanoate | 13 ± 4.7 | 1.9 ± 0.5 |
| I-23 | 3 | 2BL-CASI-C6bbGMe-2-TG-octanoate | 8.6 ± 2.1 | 1.7 ± 0.6 |
| I-9′ | 3 | 2BL-CASI-C5bcPr-2-TG-octanoate | 5.6 ± 0.2 | 1.1 ± 0.1 |
| I-19 | 3 | 2BL-FSI5-C6bbGMe-2-TG-octanoate | 22 ± 3 | 4.3 ± 0.4 |
| I-9 | 6 | 2BL-CASI-C5bbGMe-2-TG-octanoate | 17 ± 3.6 | 3.3 ± 0.4 |
| I-16 | 3 | 2BL-FSI5-C5bbGMe-2-TG-octanoate | 16 ± 2.4 | 2.8 ± 0.4 |
| I-18 | 6 | 2BL-FSI5-C5bcPr-2-TG-octanoate | 23 ± 6.1 | 4.3 ± 0.8 |
| I-7 | 3 | 2BL-CASI-C5bMe-2-TG-octanoate | 12 ± 2 | 1.9 ± 0.3 |
| 2BL | 3 | 2-Br-LSD (PO) (SEDDS Formulation) | 13 ± 3.4 | 2.1 ± 0.4 |
| 2BL | 3 | 2-Br-LSD (IV) | 300 ± 40 | 280 ± 8.8 |

TABLE 7
Plasma Exposure of 2-Bromo-LSD in Female NHPs

| | | | Plasma | Plasma |
|---|---|---|---|---|
| | | | AUC(0-last) | Cmax |
| | Sample | | (ng · h/mL) | (ng/mL) |
| ID | Size (n) | Synonyms | (±SD) | (±SD) |
| I-22 | 3 | 2BL-FSI4-C6bbGMe-2-TG-octanoate | 12 ± 2.4 | 2 ± 0.2 |
| I-23 | 3 | 2BL-CASI-C6bbGMe-2-TG-octanoate | 13 ± 4.1 | 2.2 ± 0.5 |
| I-9′ | 2 | 2BL-CASI-C5bcPr-2-TG-octanoate | 7.7 ± 0.4 | 1.1 ± 0.3 |
| I-19 | 3 | 2BL-FSI5-C6bbGMe-2-TG-octanoate | 17 ± 2.1 | 2.7 ± 0.5 |
| I-9 | 5 | 2BL-CASI-C5bbGMe-2-TG-octanoate | 20 ± 5.5 | 3.0 ± 0.7 |
| I-16 | 3 | 2BL-FSI5-C5bbGMe-2-TG-octanoate | 15 ± 2.4 | 2.3 ± 0.6 |
| I-18 | 5 | 2BL-FSI5-C5bcPr-2-TG-octanoate | 20 ± 6.1 | 3.0 ± 0.7 |
| I-7 | 2 | 2BL-CASI-C5bMe-2-TG-octanoate | 16 ± 1.2 | 2.6 ± 0.8 |
| 2BL | 2 | 2-Br-LSD (PO) (SEDDS Formulation) | 23 ± 4.2 | 3.9 ± 1.5 |
| 2BL | 2 | 2-Br-LSD (IV) | 340 ± 38 | 340 ± 58 |

TABLE 8
Plasma Exposure of Combined 2-Bromo-LSD in Male NHPs

| | | | Plasma | Plasma |
|---|---|---|---|---|
| | | | AUC(0-last) | Cmax |
| | Sample | | (ng · h/mL) | (ng/mL) |
| ID | Size (n) | Compound | (±SD) | (±SD) |
| I-22 | 3 | 2BL-FSI4-C6bbGMe-2-TG-octanoate | 82 ± 32 | 18 ± 4.1 |
| I-23 | 3 | 2BL-CASI-C6bbGMe-2-TG-octanoate | 66 ± 13 | 15 ± 3.3 |
| I-9′ | 3 | 2BL-CASI-C5bcPr-2-TG-octanoate | 56 ± 8 | 12 ± 1.8 |
| I-19 | 3 | 2BL-FSI5-C6bbGMe-2-TG-octanoate | 290 ± 58 | 51 ± 9.7 |
| I-9 | 6 | 2BL-CASI-C5bbGMe-2-TG-octanoate | 190 ± 58 | 38 ± 10 |
| I-16 | 3 | 2BL-FSI5-C5bbGMe-2-TG-octanoate | 470 ± 120 | 110 ± 40 |
| I-18 | 6 | 2BL-FSI5-C5bcPr-2-TG-octanoate | 450 ± 160 | 90 ± 35 |
| I-7 | 3 | 2BL-CASI-C5bMe-2-TG-octanoate | 47 ± 8 | 8.7 ± 2.3 |

TABLE 9
Plasma Exposure of Combined 2-Bromo-LSD in Female NHPs

| | | | Plasma | Plasma |
|---|---|---|---|---|
| | | | AUC(0-last) | Cmax |
| | Sample | | (ng · h/mL) | (ng/mL) |
| ID | Size (n) | Synonyms | (±SD) | (±SD) |
| I-22 | 3 | 2BL-FSI4-C6bbGMe-2-TG-octanoate | 74 ± 15 | 15 ± 2.1 |
| I-23 | 3 | 2BL-CASI-C6bbGMe-2-TG-octanoate | 53 ± 3.7 | 11 ± 1.4 |
| I-9′ | 2 | 2BL-CASI-C5bcPr-2-TG-octanoate | 34 ± 1.3 | 8.1 ± 0.5 |
| I-19 | 3 | 2BL-FSI5-C6bbGMe-2-TG-octanoate | 150 ± 4.6 | 29 ± 0.9 |
| I-9 | 5 | 2BL-CASI-C5bbGMe-2-TG-octanoate | 120 ± 22 | 23 ± 6 |
| I-16 | 3 | 2BL-FSI5-C5bbGMe-2-TG-octanoate | 320 ± 73 | 53 ± 8 |
| I-18 | 5 | 2BL-FSI5-C5bcPr-2-TG-octanoate | 320 ± 130 | 65 ± 15 |
| I-7 | 3 | 2BL-CASI-C5bMe-2-TG-octanoate | 49 ± 19 | 8.5 ± 0.2 |

We claim:

1. A compound of Formula I: 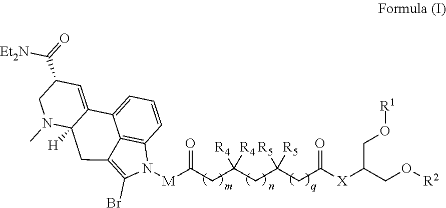
or a pharmaceutically acceptable salt thereof, wherein:
R¹ and R² are each independently hydrogen or —C(O)R³;
each R³ is independently a saturated or unsaturated, straight or branched, optionally substituted C₂₋₃₇ hydrocarbon chain;
X is —O—;
R⁴ and R⁵ are each independently hydrogen or C₁₋₄ aliphatic optionally substituted with 1, 2, 3, 4, 5, or 6 deuterium or halogen atoms, or R⁴ and R⁵ taken together with the carbon to which they are attached form a C₃-C₈ cycloalkyl;
M is absent or a self-immolative group;
n is 0-18; and
m and q are each independently 0-6.

2. The compound of claim 1, wherein R¹ and R² are each independently —C(O)R³.

3. The compound of claim 1 or 2, wherein each R³ is independently a saturated or unsaturated, straight or branched, optionally substituted C₃₋₁₇ hydrocarbon chain.

4. The compound of any one of claims 1-3, wherein each R³ is a C₇ hydrocarbon chain.

5. The compound of claim any one of claims 1-4, wherein m and q are each independently 0 or 1.

6. The compound of claim any one of claims 1-5, wherein n is 0-6.

7. The compound of any one of claims 1-6, wherein R⁴ and R⁵ are each independently hydrogen or methyl.

8. The compound of any one of claims 1-7, M is selected from

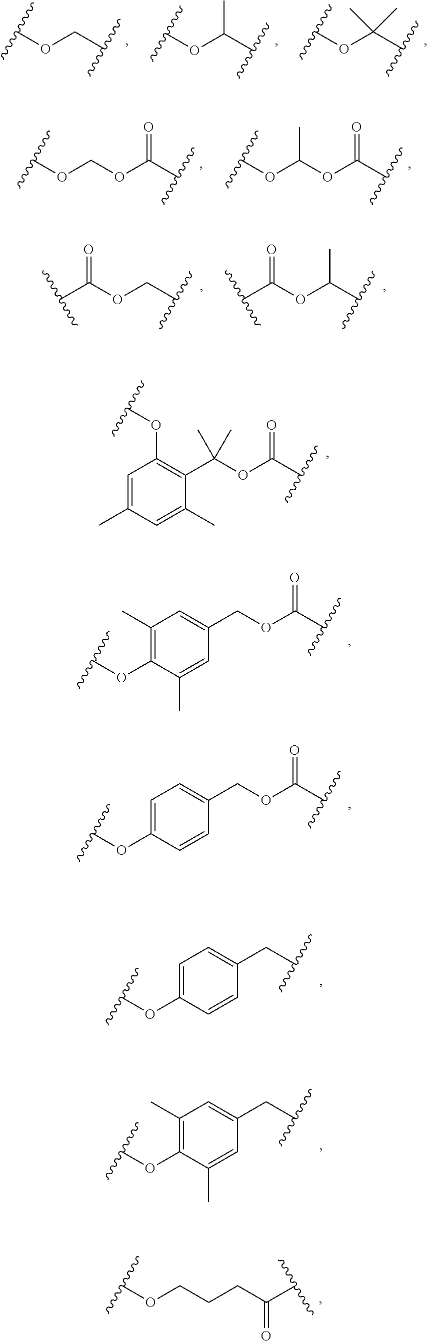

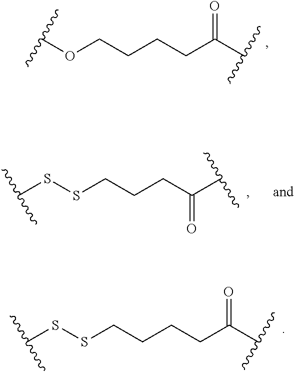 [SMILES: *OCCCCC(*)=O ; *SSCCCC(*)=O ; *SSCCCCC(*)=O]

9. The compound of any one of claims 1-8, wherein M is

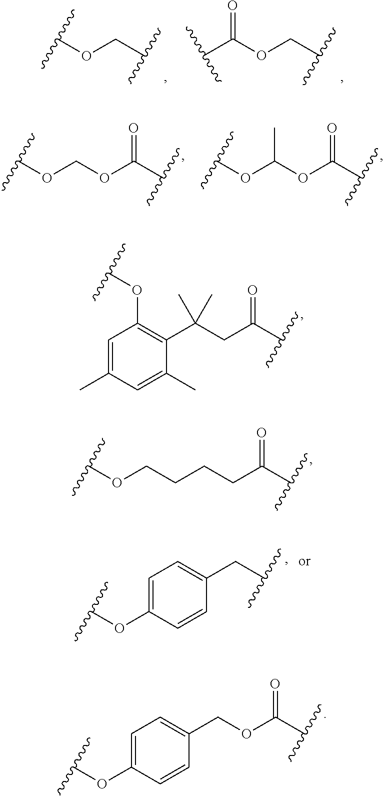

10. The compound of any one of claims 1-9, wherein the compound of Formula I is selected from Table 1.

11. A compound of Formula II: 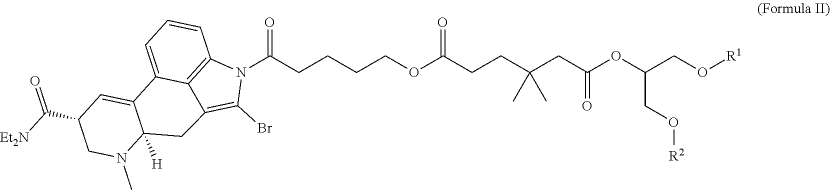
wherein R¹ and R² are each independently —C(O)R³; and
each R³ is independently a saturated or unsaturated, straight or branched, optionally substituted C₃₋₁₇ hydrocarbon chain.

12. The compound of claim 11, wherein each R³ is independently a saturated or unsaturated, straight C₃₋₁₇ hydrocarbon chain.

13. The compound of claim 11 or 12, wherein each R³ is a saturated C₇ hydrocarbon chain.

14. The compound of any one of claims 11-13, wherein the compound is

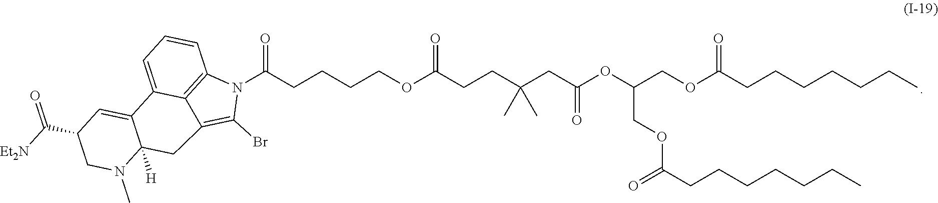

15. A compound of Formula III: 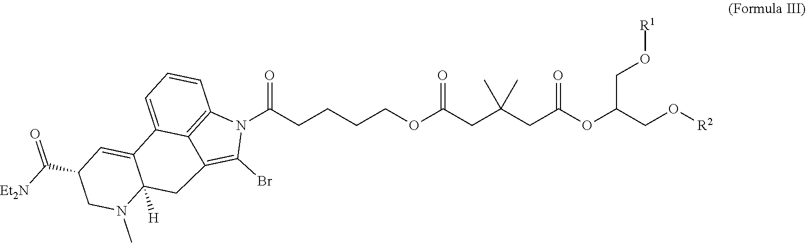
wherein R¹ and R² are each independently —C(O)R³; and
each R³ is independently a saturated or unsaturated, straight or branched, optionally substituted C₃₋₁₇ hydrocarbon chain.

16. The compound of claim 15, wherein each R³ is independently a saturated or unsaturated, straight C₃₋₁₇ hydrocarbon chain.

17. The compound of claim 15 or 16, wherein each R³ is a saturated C₇ hydrocarbon chain.

18. The compound of any one of claims 15-18, wherein the compound is:

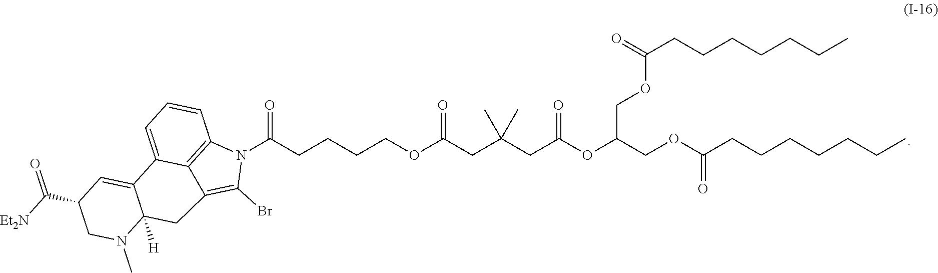

19. A compound of Formula IV: 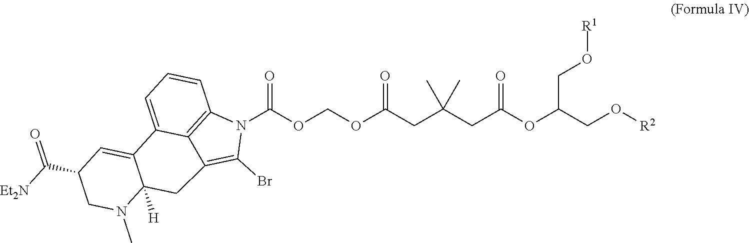
wherein R¹ and R² are each independently —C(O)R³; and
each R³ is independently a saturated or unsaturated, straight or branched, optionally substituted C₃₋₁₇ hydrocarbon chain.

20. The compound of claim 19, wherein each R³ is independently a saturated or unsaturated, straight C₃₋₁₇ hydrocarbon chain.

21. The compound of claim 19 or 20, wherein each R³ is a saturated C₇ hydrocarbon chain.

22. The compound of any one of claims 19-21, wherein the compound is:

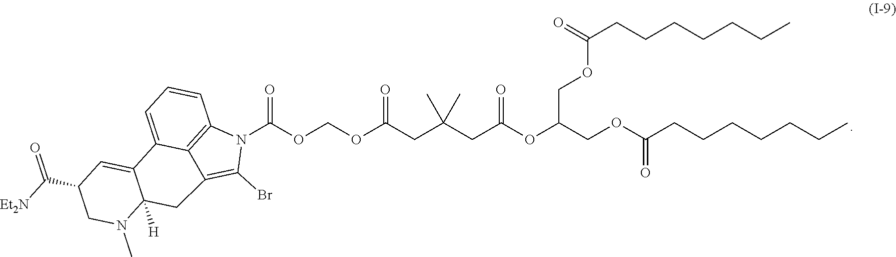

23. A compound of Formula V: 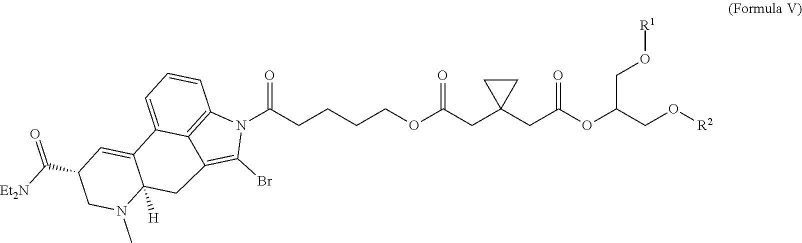
wherein R¹ and R² are each independently —C(O)R³; and
each R³ is independently a saturated or unsaturated, straight or branched, optionally substituted C₃₋₁₇ hydrocarbon chain.

24. The compound of claim 23, wherein each R³ is independently a saturated or unsaturated, straight C₃₋₁₇ hydrocarbon chain.

25. The compound of claim 23 or 24, wherein each R³ is a saturated C₇ hydrocarbon chain.

26. The compound of any one of claims 23-25, wherein the compound is:

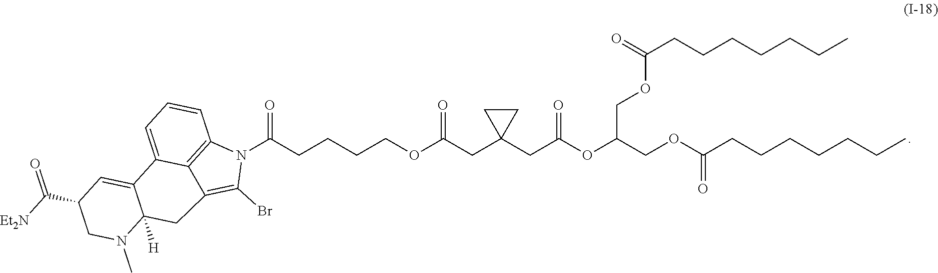

27. A pharmaceutically acceptable composition comprising a compound of any one of claims 1-26, and a pharmaceutically acceptable carrier, adjuvant, or vehicle.

28. The pharmaceutically acceptable composition of claim 27, which is administered orally to a subject in need thereof.

29. A method of treating cluster headache in a patient comprising orally administering to the patient in need thereof the compound of any one of claims 1-26, or the pharmaceutically acceptable composition of claim 27.

30. A method of treating migraine in a patient comprising orally administering to the patient in need thereof the compound of any one of claims 1-26, or the pharmaceutically acceptable composition of claim 27.

31. A method of treating a mood disorder in a patient comprising orally administering to the patient in need thereof the compound of any one of claims 1-26, or the pharmaceutically acceptable composition of claim 27.

32. The method of claim 31, wherein the mood disorder is depression, bipolar disorder, premenstrual syndrome dysphoric disorder (PMDD), intermittent explosive disorder (IED), post-traumatic stress disorder (PTSD), or substance-induced mood disorder.

33. A method of treating an anxiety disorder in a patient comprising orally administering to the patient in need thereof the compound of any one of claims 1-26, or the pharmaceutically acceptable composition of claim 27.

34. The method of any one of claims 29-33, further comprising the step of administering an additional therapeutic agent.
